SEQUENCE LISTING (2 sequences)

SEQ ID NO: 1 - DNA, 45 bp, Artificial Sequence
  Artificial oligonucleotide template
tcccgtttcgtctcgttccgcagggtcctatagtgagtcgtatta

SEQ ID NO: 2 - DNA, 19 bp, Artificial Sequence
  Artificial Oligonucleotide Primer
taatacgactcactatagg

(19) United States
(12) Patent Application Publication
(10) Pub. No.: US 2010/0304368 A1
(43) Pub. Date: Dec. 2, 2010

(54) COMPONENTS AND METHOD FOR ENZYMATIC SYNTHESIS OF NUCLEIC ACIDS

(76) Inventors: Dmitry Cherkasov, Marburg (DE); Englebert Bäuml, Gross Grönau (DE); Elisabeth Bäuml, Gross Grönau (DE)

Correspondence Address: MCDERMOTT WILL & EMERY LLP, 600 13TH STREET, N.W., WASHINGTON, DC, 20005-3096, US

(21) Appl. No.: 12/442,184

(22) PCT Filed: Sep. 20, 2007
(86) PCT No.: PCT/EP07/08198

§ 371 Date: Jul. 16, 2010

(30) Foreign Application Priority Data
Sep. 20, 2006 (DE) 10 2006 044 749.2

(51) Int. Cl. C12Q 1/68; C07H 21/04; C12P 19/34; C07H 19/20; C12N 9/96; A61K 39/395; C12N 9/12

(52) U.S. Cl. 435/6; 536/24.33; 435/91.1; 536/26.26; 435/188; 530/358; 435/194

(57) ABSTRACT

Novel methods for enzymatic synthesis of nucleic acid chains and the substrates for the same are disclosed. The methods are based on a step-wise enzymatic reaction. The sequencing of nucleic acids is an example of the use of the claimed methods.

### DESCRIPTION OF THE INVENTION

### 1.1. Technical Field

Enzymatic synthesis of nucleic acids plays an important role in modern industry. In the future, even further application fields are anticipated, e.g., in nanobiotechnology. Besides processes used already for a long time for an easy amplification of the nucleic acid chains, like PCR, processes and products that are based on a step-by-step enzymatic synthesis reaction are under development, see for instance (www.genovoxx.com; www.illumina.com; www.helicosbio.com). The application of the nucleic acids as templates for the synthesis of nanobiological complexes with multiple functions hold promise in areas like nanomedicine and very strong storage systems. The ability to control enzymatic synthesis of nucleic acids effectively is a requirement for the quality of such processes. Hence, there is a further need for means and processes which allow such control.

A great variety of chemical protective groups for nucleotides and their analogues that permit a controlled step-by-step chemical synthesis has been developed during the last 20-30 years. For example, processes for synthesis of oligonucleotides based upon them have been known for a long time. In contrast, processes based on enzymatic synthesis could barely profit from such protective groups. The relevance of this subject is pointed out by the strong support of development in this area provided by NIH in 2004 and 2005 (The National institute of Health). There, projects that endeavor to further develop modified compounds for enzymatic nucleic acid syntheses are supported.

### 1.2 Purpose of the Invention

Supply of reagents, kits and processes to control the progress of the enzymatic synthesis reaction of nucleic acid chains.
Supply of reagents, kits and processes for the sequencing of nucleic acid chains.

The new processes are characterized in that macromolecular, sterically demanding ligands are involved in the control of the enzymatic reaction. The sterically demanding ligands are coupled to the incorporated modified nucleotides and the mass of these ligands is more than 2 kDa.

### DESCRIPTION

### 1.3 Terms and Definitions

1.3.1 Macromolecular compound—a molecule or complex of molecules or a nanocrystal or nanoparticle, which has a molecular weight between 2 kDa and 100 GDa, especially in the arange between 2 kDa and 20 kDa, 2 kDa and 50 kDa, 2 kDa and 100 kDa, 100 kDa and 200 kDa, 200 kDa and 1000 kDa or 1 MDa and 100 MDa or 100 MDa and 100 Gda. Examples of macromolecular compounds are nucleic acids, e.g. oligonucleotides with a length of more than 10 nucleotides, polynucleotides, polypeptides, proteins or enzymes, quantum dots, polymers like PEG, Mowiol, dextran, polyacrylate, nanoparticel with a diameter in the range of 10 to 100 nm, 20 to 200 nm, 30 to 300 nm, 40 to 400 nm, 50 to 500 nm (e.g., Nanogold particle, Polystyrene particles, paramagnetic particles on dextran basis), microparticle with a diameter in the range of 0.5 to 1 μm, 1 to 5 μm and complexes comprising several macromolecules.

1.3.2 Low-molecular compound—a molecule or a molecule complex, which has a mass smaller than 2000 Da (2 kDa), e.g. biotin, natural nucleotides, dATP, dUTP, many dyes, like Cy3, rhodamine, fluorescein and conventionally modified nucleotides, like biotin-16-dUTP.

### 1.3.3 Nuc-Macromolecule and a Modified Nuc-Macromolecule

A Nuc-macromolecule comprises at least one nuc-component, one linker component, and at least one marker component (see also WO2005044836 and WO2006097320, the content of these applications is incorporated by reference for the purposes of USPTO for the USA).

The present invention describes modified nuc-macromolecules. One modified nuc-macromolecule is a nucleotide analog. It comprises at least one nucleotide component (nuc-component), at least one linker component, at least one marker component and at least one macromolecular, sterically demanding ligand (in the further course of the description, such molecules will be called “modified nuc-macromolecules”; some examples are depicted schematically in FIGS. 1 and 2).

A) (Nuc-Linker 1)ₙ-(Ligand)ₖ-(Marker)ₘ
or
B) (Nuc-Linker 1)ₙ-(Ligand-Linker 3)ₖ-(Marker)ₘ
or
C) (Nuc-Linker 1)ₙ-(Ligand)ₖ-(Linker 3-Marker)ₘ
or
D) (Nuc-Linker 1)ₙ-(Marker)ₘ-(Ligand)ₖ
or

### E) (Nuc-Linker 1-Ligand)ₙ-(Marker)ₘ

or
F) (Ligand-Linker 2-Nuc-Linker 1)ₙ-(Marker)ₘ
or
G) (Nuc-Linker 1)ₙ-(Marker/Ligand)ₘ
wherein:
Nuc—is a nuc-component
Linker—is a linker component, wherein linker 1 or linker 2 or linker 3 can have identical or different structures
Marker—is a marker component
Ligand—is a macromolecular sterically demanding ligand
Marker/ligand—is a structure that has properties both of a marker and of a macromolecular, sterically demanding ligand
n—is a positive integer from 1 to 100000
m—is a positive integer from 1 to 1000
k—is a positive integer from 1 to 1000

In one embodiment, the structure comprises the following distribution within the molecule: (n)≧(m)≧(k), wherein individual numbers can be varied independently of one another. In a further embodiment, the structure comprises the following distribution: (n)>(m)>(k), wherein individual figures can be varied independently of one another. Further combinations of the components of the nuc-macromolecules should be obvious for a person skilled in the art.

In one embodiment of the invention, the linker is water-soluble. Its composition is not restricted as long as substrate properties of the nucleotides are not lost. Its length ranges between 5 and 100,000 atoms.

In a further embodiment, the linker component comprises a coupling unit (L) for coupling the linker to the nuc-component, a water soluble polymer and a coupling unit (T) for coupling the linker to the marker component. In this preferred embodiment, a modified nuc-macromolecule has the following structure:

### (Nuc-L-Polymer-T)ₙ-Ligand-Marker

or

### (Nuc-L-Polymer-T)ₙ-Marker-Ligand

wherein:
Nuc—is a nucleotide monomer or a nucleoside monomer (nuc-component)
L—is a part of the linker that represents a linkage between nuc and the rest of the linker (coupling unit L)
T—is a part of the linker that represents a linkage between the rest of the linker and the marker (coupling unit T)
Polymer—is a part of the linker that is a water-soluble polymer with an average length between 5 and 100,000 atoms. (In this embodiment, the coupling unit (L), the polymer and the coupling unit (T) are combined as the linker component)
Marker—is a marker component
Ligand—is a macromolecular sterically demanding ligand
n—is a positive integer from 1 to 1000000, wherein (n) can represent an average number.

### 1.3.3.1 Nucleotide-Component or Nuc-Component

The nuc-component is a modified nucleotide and is a component of a modified nuc-macromolecule and has substrate properties for polymerases.

The nuc-component preferably comprises a base part (base), a sugar part (sugar) and optionally a phosphate part (phosphate). Base, sugar and phosphate can be modified, i.e. the basic structure resembles the natural occurring nucleotides, but comprises e.g. additional chemical groups. Examples for combinations of different nucleotide components are known to the person skilled in the art. Such nuc-components can be used in a variety of enzymatic and chemical reactions (G. Wright et al. Pharmac. Ther. 1990, v. 47, p. 447-).

In one embodiment, the nuc-component is a nucleotide monomer, which is coupled to the linker component. In principle, all conventional nucleotide variants that are suitable as a substrate for nucleotide-accepting enzymes can serve as nuc-component of the modified nuc-macromolecule so that naturally occurring nucleotides as well as modified nucleotides (nucleotide analogs) can be considered for the nuc-component. Modified nucleotides comprise base-, sugar- or phosphate-modified nucleotide analogs, FIG. 3. Many examples are known to the person skilled in the art (“Advanced organic chemistry of nucleic acids”, 1994, Shabarova, ISBN 3-527-29021-4, “Nucleotide Analogs” Scheit, 1980, ISBN 0-471-04854-2, “Nucleoside and Nucleic Acid Chemistry”, Kisakürek 2000, “Anti-HIV Nucleosides” Mitsuya, 1997, “Nucleoside Analogs in cancer therapy”, Cheson, 1997); further examples for modifications of the nucleotides will also be cited in the text.

### 1.3.3.1.1 Variations of the Phosphate

In one embodiment the nuc-component is a nucleoside-triphosphate. Still higher numbers of phosphate groups in a nucleotide (tetraphosphate etc.) can be used. Optionally, the phosphate part of the nucleotide can comprise modifications, in one embodiment such modifications comprising a linker, for example (D. Jameson et al. Methods in Enzymology 1997, v. 278, p. 363-, A. Draganescu et al. J. Biol. Chem. 2000 v. 275, p. 4555-). In another embodiment of the invention, the phosphate part of the nuc-component comprises thiotriphosphate derivates (Burges et al. PNAS 1978 v. 75, p. 4798-).

The said phosphate modifications can be located at the 5′-position of the sugar, like nucleoside-triphosphates, or also at other positions of the sugar part of the nucleotide, e.g. at the 3′-position.

### 1.3.3.1.2 Variations of the Base

The nuc-component can be nucleotide or nucleoside occurring in the nucleic acids in nature or their analogs, preferably participating at the Watson-Crick base-pairing, e.g. adenine, guanine, thymine, cytosine, uracil, inosine or modified bases like 7-deazaadenine, 7-deazaguanine, 6-thioadenine (as referred above). Optionally, the base comprises modifications. In one embodiment, such modifications comprise for example a linker, e.g. amino-propargyl-linker or amino-allyl-linker. Further examples of linkers are known (Ward et al. U.S. Pat. No. 4,711,955, G. Wright et al. Pharmac. Ther. 1990, v. 47, p. 447-, Hobbs et al. U.S. Pat. No. 5,047,519 or other linkers e.g. Klevan U.S. Pat. No. 4,828,979, Seela U.S. Pat. No. 6,211,158, U.S. Pat. No. 4,804,748, EP 0286028, Hanna M. Method in Enzymology 1996 v. 274, p. 403, Zhu et al. NAR 1994 v. 22 p. 3418, Jameson et al. Method in Enzymology, 1997, v. 278, p. 363-, Held et al. Nucleic acid research, 2002, v. 30 p. 3857-, Held et al. Nucleosides, nucleotides & nucleic acids, 2003, v. 22, p. 391, Short U.S. Pat. No. 6,579,704, Odedra WO 0192284). In one embodiment, a linker coupled to the base represents a connection part between the nuc-component and the linker component of the modified nuc-macromolecule. Further modifications of the base are described for example in the catalogue of Trilink Biotechnologies, Inc. San Diego, USA, Issue 2003, page 38.

Different variations of the sugar part of the nucleotides, which are used e.g. in the diagnostics, therapy or research, are known to the person skilled in the art. Such variations comprise ribose, 2′-deoxyribose or 2′,3′-dideoxyribose. Optionally, the sugar part comprises modifications (M. Metzker et al. Nucleic Acid Research 1994, v. 22, p. 4259-, Tsien WO 91/06678). In one embodiment, such modifications comprise for example a linker. The modifying group can be optionally be reversibly coupled to the sugar part (WO2007053719, Hovinen et al. J. Chem. Soc. Prking Trans. 1994, s. 211-, Canard U.S. Pat. No. 5,798,210, Kwiatkowski U.S. Pat. No. 6,255,475, Kwiatkowski WO 01/25247, Ju et al. U.S. Pat. No. 6,664,079, Fahnestock et al. WO 91066678, Cheeseman U.S. Pat. No. 5,302,509, Parce et al. WO 0050642, Milton et al. WO 2004018493, Milton et al. 2004018497, at the 2′-OH-position (WO2007075967)). These applications are incorporated here by reference.

In one embodiment, the linker coupled to the sugar part represents the connection between the nuc-component and the linker component of the modified nuc-macromolecules.

In another embodiment, the sugar part comprises for example the following modifications: optionally the 3′-OH-Group or the 2′-OH-Group can be substituted by the following atoms or groups: halogen atoms, hydrogen atoms, amino- or mercapto- or azido groups (Beabealashvilli et al. Biochem Biophys Acta 1986, v. 868, p. 136-, Yuzhanov et al. FEBS Lett. 1992 v. 306, p. 185-).

In another embodiment, the nuc-component comprises acyclic nucleotide or nucleoside modifications (A. Holy Current Pharmaceutical Design 2003 v. 9, p. 2567-, G. Wright et al. Pharmac. Ther. 1990, v. 47, p. 447-). In another embodiment, the sugar part comprises a double bond.

In this application, the following abbreviations will be used for 2′-deoxynucleotides: dUTP for 2′-deoxyuridine-triphosphate, dCTP for 2′-deoxycytidine-triphosphate, dATP for 2′-deoxyadenosine-triphosphate, dGTP for 2′-deoxyguanosine-triphosphate.

### 1.3.3.1.4 Linking of the Nucleotide and Linker

The nuc-component is linked to the linker at a coupling position. This coupling position of the linker on the nuc-component can be located on the base, on the sugar (e.g. ribose or deoxyribose) or on the phosphate part. Several linkers can be coupled to the one nuc-component (see linker description).

The linkage between the linker component and the nuc-component is preferably covalent.

If the coupling position is on the base, then the following positions are preferable: position 4 or 5 for pyrimidine bases and positions 6, 7, 8 for purine bases. (Ward et al. U.S. Pat. No. 4,711,955, G. Wright et al. Pharmac. Ther. 1990, V. 47, S. 447-, Hobbs et al. U.S. Pat. No. 5,047,519 oder andere Linker z. B. Klevan U.S. Pat. No. 4,828,979, Seela U.S. Pat. No. 6,211,158, U.S. Pat. No. 4,804,748, EP 0286028, Hanna M. Method in Enzymology 1996 v. 274, S.403, Zhu et al. NAR 1994 v. 22 S.3418, Jameson et al. Method in Enzymology, 1997, v. 278, S. 363-, Held et al. Nucleic acid research, 2002, v. 30 3857-, Held et al. Nucleosides, nucleotides & nucleic acids, 2003, v. 22, S. 391, Short U.S. Pat. No. 6,579,704, Odedra WO 0192284). On sugar, positions 2′, 3′, 4′ or 5′ can serve as coupling positions. The coupling to the phosphate groups can proceed via alpha, beta, or gamma phosphate groups. Examples for coupling positions on the base are described in Short WO 9949082, Balasubramanian WO 03048387, Tcherkassov WO 02088382 (also see commercially available nucleotides e.g. from Amersham or Roche), on the ribose in Herrlein et al. Helvetica Chimica Acta, 1994, v. 77, p. 586, Jameson et al. Method in Enzymology, 1997, v. 278, p. 363, Canard U.S. Pat. No. 5,798,210, Kwiatkowski U.S. Pat. No. 6,255,475, Kwiatkowski WO 01/25247, Parce WO 0050642, on phosphate groups in Jameson et al. Method in Enzymology, 1997, v. 278, p. 363.

The location of the coupling position depends on the area of application of the modified nuc-macromolecules. For example, coupling positions on the sugar or on the base are preferable in cases where the marker is intended to stay coupled to the nucleic acid strand. The coupling to the gamma or beta phosphate groups can be used for example in cases where the marker has to be separated during the incorporation of the modified nuc-macromolecule.

The linking between the nuc-component and the linker component results for example via a coupling unit (L) that is a part of the linker component.

In one embodiment, the linkage between the nuc-component and the linker is stable, e.g. resistant to temperatures up to 130° C., pH-ranges from 1 to 14 and/or resistant to hydrolytical enzymes (e.g. proteases or esterases). In another embodiment of the invention, this linkage between the nuc-component and the linker component is cleavable under mild conditions.

This cleavable linkage allows removal of the linker components and the marker components. In one embodiment of the invention, it allows removal of the sterically demanding ligand, too. This can be advantageous for example for methods of sequencing by synthesis, like pyrosequencing, BASS (base addition sequencing schema) (Canard et al. U.S. Pat. No. 5,798,210, Rasolonjatovo Nucleosides & Nucleotides 1999, v. 18, p. 1021, Metzker et al. NAR 1994, v. 22, p. 4259, Welch et al. Nucleosides & Nucleotides 1999, v. 18, p. 19, Milton et al. WO 2004018493, Odedra at al. WO 0192284) or single molecule sequencing Tcherkassov WO 02088382. The choice of the cleavable linkage is not restricted insofar as it remains stable under conditions of enzymatic reaction, does not result in irreversible damage of the enzyme (e.g. polymerase) and is cleavable under mild conditions. “Mild conditions” is understood to mean conditions that do not result in damage of nucleic acid-primer complexes wherein, for example, the pH-range is preferably between 3 and 11 and the temperature is between 0° C. and the temperature value (x). This temperature value (x) is dependent upon the Tm of the nucleic acid-primer complex (where Tm is the melting temperature) and is calculated for example as Tm (nucleic acid primer complex) minus 5° C. (e.g. Tm is 47° C., then the (x)-value is 42° C.; ester, thioester, acetales, phosphoester, disulfide linkages and photolabile compounds are suitable as cleavable linkages under these conditions).

Preferably, the said cleavable linkage comprises chemical or enzymatic cleavable linkages or photolabile compounds. Ester, thioester, disulfide and acetal linkages are examples of chemical cleavable groups (Short WO 9949082, “Chemistry of protein conjugation and crosslinking” Shan S. Wong 1993 CRC Press Inc., Herman et al. Method in Enzymology 1990 v. 184 p. 584, Lomant et al. J. Mol. Biol. 1976 v. 104 243, “Chemistry of carboxylic acid and esters” S. Patai 1969 Interscience Publ.). Examples for photolabile compounds are described in Rothschild WO 9531429, “Protective groups in organic synthesis” 1991 John Wiley & Sons, Inc., V. Pillai Synthesis 1980 p. 1, V. Pillai Org. Photochem. 1987 v. 9 p. 225, Dissertation “Neue photolabile Schutzgruppen für die lichtgesteuerte Oligonucleotidsynthese” H. Giegrich, 1996, Konstanz, Dissertation “Neue photolabile Schutzgruppen für die lichtgesteuerte Oligonucleotidsynthese” S. M. Bühler, 1999, Konstanz). Still further cleavable groups used for nucleotide chemistry are described in Milton et al. WO2004018493, Milton et al. WO2004018497.

### 1.3.3.1.5 Number of the Linked Nuc-Components

In one embodiment of the invention, only one nuc-component is coupled per modified nuc-macromolecule. In another embodiment of the invention, several nuc-components are coupled per one modified nuc-macromolecule. If several nuc-components are coupled, they can be identical or different, whereas the average number of the nuc-components per modified nuc-macromolecule can range for example from 2 to 5, 5 to 10, 10 to 25, 25 to 50, 50 to 100, 100 to 250, 250 to 500, 500 to 1000, 1000 to 10000, 10000 to 100000 or even more.

### 1.3.3.2 Linker-Component

The terms “linker” and “linker component” will be used synonymously in this application and comprise the whole structural part of the modified nuc-macromolecule between the nuc-component and the marker component or between the nuc-component and the macromolecular sterically demanding ligand or between the macromolecular sterically demanding ligand and the marker.

A distinction will be made between linkers that are linked to a nuc-component (linker 1 and linker 2) and linker (3), which links other components of modified nuc-macromolecules (e.g., sterically demanding ligand(s) and the marker(s)).

Linker 3 can be composed in analogous way like linker 1 and 2 or have another structure. The composition of linker 3 is not limited, as long as it does not destroy the enzymatic properties of the modified nuc-macromolecule and prevent the enzymatic reaction.

In the following, linker 1 and 2 will be discussed in detail. A general term “linker” will be used since only one linker component is linked to the nuc-component in most embodiments.

The linker is preferably water-soluble. The precise linker composition is not limited and can vary.

The length of linker is considered as the shortest distance (theoretically calculated on the stretched status of the linker) from the nuc-component to the next macromolecular structure (e.g., macromolecular sterically demanding ligand or macromolecular marker). Exemplarily the distance is calculated to the marker or to the steric obstacle.

In a preferred embodiment, modified nuc-macromolecules have a short linker. Its length is between 2 and 30 chain atoms. Such linkers can carry functional groups, as for example amino, carboxy, mercapto and hydroxy groups. Further molecules, e.g., macromolecules, like water-soluble polymers, can be coupled to these groups. Examples of short linkers coupled to the nucleotides are known to the person skilled in the art. (Ward et al. U.S. Pat. No. 4,711,955, G. Wright et al. Pharmac. Ther. 1990, V. 47, p. 447-, Hobbs et al. U.S. Pat. No. 5,047,519 or other linkers e.g. Klevan U.S. Pat. No. 4,828,979, Seela U.S. Pat. No. 6,211,158, U.S. Pat. No. 4,804,748, EP 0286028, Hanna M. Method in Enzymology 1996 v. 274, p. 403, Zhu et al. NAR 1994 v. 22 p. 3418, Jameson et al. Method in Enzymology, 1997, v. 278, p. 363-, Held et al. Nucleic acid research, 2002, v. 30 3857-, Held et al. Nucleosides, nucleotides & nucleic acids, 2003, v. 22, p. 391, Short U.S. Pat. No. 6,579,704, Odedra WO 0192284). The linker can contain one or several units of water-soluble polymers, as for example amino acids, sugars, PEG units or carboxylic acids. The coupling unit (L) of a long linker can serve as further examples of short linkers (see below). Linkers with lengths between 2 and 20 atoms are preferably used in modified nuc-macromolecules whose marker component comprises linear water-soluble polymers.

In another preferred embodiment of the invention, a long linker having a length of more than 30 chain atoms is used.

Examples for the composition of the linker will now be presented below.

### 1.3.3.2.1 Parts of the Linker

### (Described Using the Example of a Linker Between the Nuc-Component and the Marker Component or Between the Nuc-Component and the Sterically Demanding Ligand).

The linker is a part of the nuc-macromolecule between the corresponding nuc-component and marker component.

The linker comprises for example the following parts in its structure:

1) coupling unit (L)
2) water soluble polymer
3) coupling unit (T)

The subdivision of the linker in separate parts is purely functional and should serve merely for better understanding of the structure. Depending on the approach, particular structures can be considered as one functional part or as another.

The coupling unit (L) has the function of linking the linker component and the nuc-component. Short, non-branched compounds from 1 to 20 atoms in length are preferred. The particular structure of the coupling unit (L) depends on the coupling position of the linker to the nucleotide or nuc-unit and on the particular polymer of the linker. Several examples of coupling units (L) are shown in examples of this application. Many conventionally modified nucleotides comprise a short linker; these short linkers are further examples of coupling units (L), e.g. short linker on the base: Short WO 9949082, Balasubramanian WO 03048387, Tcherkassov WO 02088382 (see also commercially available nucleotides from e.g. Amersham or Roche), short linker on the ribose as described in Herrlein et al. Helvetica Chimica Acta, 1994, v. 77, p. 586, Jameson et al. Method in Enzymology, 1997, v. 278, p. 363, Canard U.S. Pat. No. 5,798,210, Kwiatkowski U.S. Pat. No. 6,255,475, Kwiatkowski WO 01/25247, Ju et al. U.S. Pat. No. 6,664,079, Parce WO 0050642, and short linker on phosphate groups as described in Jameson et al. Method in Enzymology, 1997, v. 278, p. 363.

Still further examples for the coupling unit (L) are presented in the following:
R₆—NH—R₇, R₆—O—R₇, R₆—S—R₇, R₆-SS-R₇, R₆—CO—NH—R₇, R₆—NH—CO—R₇, R₆—CO—O—R₇, R₆—O—CO—R₇, R₆—CO—S—R₇, R₆—S—CO—R₇, R₆—P(O)₂—R₇, R₆—Si—R₇, R₆—(CH₂)ₙ—R₇, R₆—(CH₂)ₙ—R₇, R₆-A-(CH₂)ₙ—R₇, R₆—(CH₂)ₙ—B—R₇, R₆—(CH═CH—)ₙ—R₇, R₆-(A-CH═CH—)ₙ—R₇, R₆—(CH═CH—B—)ₙ—R₇, R₆-A-CH═CH—(CH₂—)ₙ—R₇, R₆—(—CH═CH—CH₂)ₙ—B—R₇, R₆—(—CH═CH—CH₂—CH₂)ₙ—B—R₇, R₆—(C≡C—)ₙ—R₇, R₆-(A-C≡C—)ₙ—R₇, R₆—(c≡C—B—)ₙ—R₇, R₆-A-C≡C—(CH₂—)ₙ—R₇, R₆—(—C≡CH₂)ₙ—B—R₇, R₆—(—C≡C—CH₂—CH₂)ₙ—B—R₇,
where R₆ is the nuc-component; R₇ is a polymer; A and B comprises the following structural elements: —NH—, —O—, —S—, -SS-, —CO—NH—, —NH—CO—, —CO—O—, —O—CO—, —CO—S—, —S—CO—, —P(O)₂—, —Si—, —(CH₂)ₙ—, a photolabile group; (n) is a number from 1 to 5

The coupling unit L is linked to the nuc-component on the one side and to the polymer on the other. The character of the linkage with the polymer depends on the kind of polymer. In a preferred embodiment, the ends of the polymer comprises reactive groups, for example NH2 (amino), OH (hydroxy), SH (mercapto), COOH (carboxy), CHO (aldehyde), acrylic, maleimide or halogen groups. Such polymers are commercially available (e.g. Fluka). Some examples for the coupling of polymers to the coupling unit are shown in the examples.

In a preferred embodiment, the water-soluble polymer represents the major part of the linker component. It is a polymer, preferably hydrophilic, consisting of the same or different monomers. Examples of suitable polymers are polyethylene-glycol (PEG), polyamides (e.g. polypeptides), polysaccharides and their derivates, dextran and its derivates, polyphosphates, polyacetates, poly(alkyleneglycols), copolymers with ethylenglycol and propyleneglycol, poly(olefinic alcohols), poly(vinylpyrrolidones), poly(hydroxyalkylmethacrylamides), poly(hydroxyalkylmethacrylates), poly(x-hydroxy acids), polyacrylic acid and their derivates, poly-acrylamide and its derivates, poly(vinylalcohol), polylactic acid, polyglycolic acid, poly(epsilon-caprolactones), poly(beta-hydroxybutyrates), poly(beta-hydroxyvalerate), polydioxanones, poly(ethylene terephthalates), poly(malic acid), poly(tartronic acid), poly(ortho esters), polyanhydrides, polycyanoacrylates, poly(phosphoesters), polyphosphazenes, hyaluronidate, and polysulfones.

In one embodiment, the polymer-part comprises branched polymers. In an other embodiment, the polymer-part comprises non-branched or linear polymers. The polymer can consist of several parts of different length, each part consisting of the same monomers with the monomers in different parts being different. To a person skilled in the art, it should seem obvious that for a macromolecular linker, it is often possible to determine only an average mass, so that the data regarding the mole masses represent an average (“Makromoleküle, Chemische Struktur and Synthesen”, Volume 1, 4, H. Elias, 1999, ISBN 3-527-29872-X). For this reason, often there is no exact mass information for modified nuc-macromolecules.

In one preferred embodiment, the linker component comprises a linear, non-branched polymer that is not modified with further sterically demanding chemical structures such as dyes, fluorescent dyes, or ligands. Such linker components lead to a low sterical hindrance, e.g. in an enzymatic recognition of the nuc-components.

In another preferred embodiment, the polymer of the linker component is linear but the linker component is modified with one or several sterically demanding chemical groups, for example dyes with low molecular weight.

Further examples of sterically demanding groups are shown in the paragraph 1.3.19.

Sterically demanding ligands or structures can be coupled to different linker parts (see paragraph 1.3.19 “Sterically demanding ligand”). The average number of the sterically demanding ligands coupled to the linker can vary and equals, for instance, between 1 and 3, 3 and 5, 5 and 20, or 20 and 50. In the coupling of sterically demanding groups, it is necessary to take into consideration that a space-demanding structure coupled in the direct proximity of the nucleotide component can lead to the loss of the substrate properties. Sterically demanding ligands can be coupled uniformly or randomly over the entire length of the linker, or they can be coupled to the linker at a certain distance from the nuc-component. The shortest distance between the nuc-component and the macromolecular steric ligand equals, for instance, 10 to 15, 15 to 20, 20 to 25, 25 to 30, 30 to 35, 35 to 40, 40 to 45, 45 to 50, 50 to 55, 55 to 60, 60 to 70, 70 to 80, 80 to 90, 90 to 100, 100 to 200, 200 to 1000, 1000 to 5000, or 5000 to 10000 chain atoms.

The sterically demanding group can be considered as a part of the linker or as a part of the marker. Which way to consider it can depend, for instance, on whether or not the sterically demanding group possesses certain signal properties.

### 1.3.3.2.2 Linker Length

### (Described Using the Example of Linker Between the Nuc-Component and the Next Macromolecular Structure, Like the Sterically Demanding Ligand or Macromolecular Marker).

An average linker length amounts to between 5 to 10, 10 to 20, 20 to 30, 30 to 40, 40 to 50, 50 to 60, 60 to 70, 70 to 80, 80 to 90, 90 to 100, 50 to 100, 100 to 200, 200 to 500, 500 to 1000, 1000 to 2000, 2000 to 10000, 10000 to 100000 atoms (chain atoms), so that an average linker length amounts to between 0.5 nm to 1 nm, 1 nm to 2 nm, 2 nm to 3 nm, 3 nm to 4 nm, 4 nm to 5 nm, 5 nm to 6 nm, 6 nm to 7 nm, 7 nm to 8 nm, 8 nm to 9 nm, 9 nm to 10 nm, 5 nm to 10 nm, 10 nm to 20 nm, 20 nm to 50 nm, 50 nm to 100 nm, 100 nm to 200 nm, 200 nm to 1000 nm (measured on a molecule potentially stretched-out as much as possible).

Since a modified nuc-macromolecule can comprise several nuc-components and therefore also several linkers, these linkers (i.e. variations of linker 1) can be of the same or different length. The values for the linker's length presented above indicate the shortest linker within the whole modified nuc-macromolecule.

Some parts of the linkers can comprise rigid areas and other parts can comprise flexible areas.

### 1.3.3.2.3 Linker Coupling in a Modified Nuc-Macromolecule (Example of the Coupling Between the Nuc-Component and the Marker Component)

The linker is connected to the nuc-component on one side and to the marker component on the other side. The linker can have coupling units at his ends which fulfill this connecting function. The connection to the nuc-component was discussed above. The connection between the linker and the marker components is provided by coupling unit T. Short, non-branched connections no more than 20 atoms in the length are preferred. The respective structure of the coupling unit T depends upon the coupling position on the marker component and upon the respective polymer of the linker.

The coupling unit T is covalently connected to the polymer. The kind of the coupling depends on the kind of the polymer. In a preferred embodiment, the polymer has reactive groups, such as NH2 (amino), OH (hydroxy), SH (mercapto), COOH (carboxy), CHO (aldehyde), acrylic, maleimide or halogen groups, at its ends. Such polymers are commercially available (e.g. Fluka). Some examples of the coupling units L are shown in examples. For further examples of the chemical and affine connections please refer to the literature: “Chemistry of protein conjugation and crosslinking” Shan S. Wong in 1993, “Bioconjugation: protein coupling techniques for the biomedical sciences”, M. Aslam, in 1996.

The linker can also comprise other functional groups or parts, for example one or several groups that are cleavable under mild conditions, see examples in WO2005044836.

A cleavable group within the linker allows the removal of a part of the linker and the marker component. After a cleavage reaction, a linker residue remains coupled to the nuc-component. Examples of cleavable groups are shown in Section 1.3.3.1.4.

### 1.3.3.3 Marker Component

The marker component can comprise different structures. The structures individually are not limited, as long as they do not destroy the substrate properties of the nuc-components for enzymes. In preferred embodiments, such structures have a signal-giving or a signal-transmitting function. The marker can also comprise other functions, for instance, structural, anti-toxic or affine function (for instance, as part of medicines or medical preparations).

### 1.3.3.3.1 The Composition of the Marker Component (Marker)

In one embodiment, the marker comprises a low-molecular marker unit. In an other embodiment, the marker comprises a macromolecular marker unit. In a still further embodiment, the marker comprises several low-molecular marker units. In a still further embodiment, the marker comprises several macromolecular marker units. In a still further embodiment, the marker comprises a combination of low-molecular and macromolecular units. The marker units can have a signal-giving or signal-transmitting function.

These units can be molecules with low molecular mass, e.g. less than 2000 Da, or they can be also macromolecules. The number of the signal-giving or signal-transmitting units, which are combined into one marker component, comprises the following ranges: 1 and 2, 2 to 5, 5 to 20, 20 to 50, 50 to 100, 100 to 500, 500 to 1000, 1000 to 10000, 10000 to 100000.

If several marker units are combined into one marker component, then in one embodiment these units are bound to a framework, the core component of the marker (FIG. 4b, c). This core component connects the units together. The core component can provide the connection to one or several nuc-linker components (FIG. 5). The core component comprises low-molecular or macromolecular compounds.

### 1.3.3.3.2 Structure of the Signal-Giving or the Signal-Transmitting Units of the Marker

The structural marker units comprise the following groups:

1.3.3.3.2.1 Structures with Low Molar Mass:

Biotin molecules, hapten molecules (e.g. digoxigenin), radioactive isotopes (e.g., P³², J¹³¹), or their derivatives, rare earth elements, dyes, fluorescent dyes, quencher of the fluorescence (e.g. dabsyl) (many of these molecules are commercially available, e.g., from Molecular Probes, Inc or from Sigma-Aldrich) with the same or different spectral properties, groups of dyes undergoing FRET. Thermochromatic, photochromatic or chemoluminescent substances are available for example from Sigma-Aldrich, chromogenic substances are described for example as substrates for peptidases in “Proteolytic enzymes Tools and Targets”, E. Sterchi, 1999, ISBN 3-540-61233-5).

Also chemically reactive groups, as for example amino-, carboxy-, merkapto-, aldehyde, iodine acetate, acrylic, dithio-, thioester-groups, can serve as signal-transmitting structural units (FIG. 6a). These reactive groups can be modified with signal-giving elements, such as dyes with suitable reactive groups (for instance, NHS esters, mercapto-, amino groups) (FIG. 6b), e.g. after incorporation of nuc-macromolecules. General rules for the choice of a suitable pair of reactive groups are shown in “Chemistry of protein conjugation and crosslinking” Shan S. Wong 1993.

In a special embodiment, a combination comprising one nuc-component, one macromolecular linker component and one marker component with a low molecular weight already fulfils the requirements of the present invention. Such compounds are also subject matter of this invention. They can be used both as intermediate compounds for the chemical synthesis of modified nuc-macromolecules with one macromolecular marker, e.g., dUTP-PEG-biotin, and as independent compounds for enzymatic reactions, as, for example, nucleotides labeled with only one dye.

Different fluorescent dyes can be used, and their choice is not limited as long as their influence of the enzymatic reaction is not substantial. Examples of such dyes are Rhodamine (Rhodamine 110, Tetramethylrhodamine, available from Fluka-Sigma), cyanine dyes (Cy2, Cy3, Cy3.5, Cy5, Cy5.5, Cy7 available from Amersham Bioscience), coumarine, Bodipy, fluorescein, Alexa Dyes: e.g., Alexa 532, Alexa 548, Alexa 555 (Molecular Probes). Many dyes are commercially available, for instance, from Molecular Probes Europe, Leiden, the Netherlands (hereinafter called Molecular Probes) or from Sigma-Aldrich-Fluka (Taufkirchen, Germany).

Examples of the synthesis of a nuc-macromolecule with a low-molecular marker are given in WO2005044836.

In one embodiment, the marker comprises several marker units. These marker units can have the same or different properties. For instance, fluorescent dyes with different spectral qualities can be used. In one embodiment, the fluorescent dyes that can form FRET pairs are selected.

1.3.3.3.2.2 Structures with High Mass (Macromolecules)

### 1.3.3.3.2.2.1 Nanocrystals

Nanocrystals, e.g. quantum dots, can serve as marker units. Quantum dots with the same or different spectral qualities can be used within the same marker component. Examples of quantum dots are presented in U.S. Pat. No. 6,322,901, U.S. Pat. No. 6,423,551, U.S. Pat. No. 6,251,303, U.S. Pat. No. 5,990,479.

### 1.3.3.3.2.2.2 Nano- or Micro-Particles

Nano- or micro-particles can serve as marker units. The diameters of these particles can range from 1 nm to 2 nm, from 2 nm to 5 nm, from 5 nm to 10 nm, from 10 nm to 20 nm, from 20 nm to 50 nm, from 50 nm to 100 nm, from 100 nm to 200 nm, from 200 nm to 500 nm, from 500 nm to 1000 nm, from 1000 nm to 5000 nm. The material of these particles can, for instance, be pure metals such as gold, silver, aluminum (as instances of particles capable of surface plasmon resonance), Protein-gold_conjugates: J. Anal. Chem. 1998; v. 70, p. 5177-, Nucleic acid-gold_conjugates: J. Am. Chem. Soc. 2001; v. 123, p. 5164-, J. Am. Chem. Soc. 2000; v. 122, p. 9071-, Biochem. Biophys. Res. Commun 2000; v. 274, p. 817-, Anal. Chem. 2001; v. 73, p. 4450-, latex (e.g., Latex-Nano-particles), Anal. Chem. 2000; v. 72, p. 1979-, plastic (Polystyrene), paramagnetic compounds: Zhi Z L et al. Anal. Biochem, 2003; v. 318 (2): p. 236-43, Dressman D et al. Proc Natl Acad Sci U.S.A. 2003, v. 100 (15): p. 8817-22, metal particles, magnetic compounds: Jain K K. Expert Rev Mol. Diagn. 2003; v. 3 (2): p. 153-61, Patolsky F et al. Angew Chem Int Ed Engl 2003; v. 42 (21), p. 2372-2376, Zhao X et al. Anal Chem. 2003; v. 75 (14): p. 3144-51, Xu H et al. J Biomed Mater Res. 2003 Sep. 15; v. 66A(4): p. 870-9, Josephson U.S. Patent No. 2003092029, Kliche WO0119405.

Several of these components are available from commercial vendors, e.g. from Miltenyi Biotech (e.g. paramagnetic particle, “Streptavidin Microbeads”) or from Sigma-Aldrich or BD Biosciences.

### 1.3.3.3.2.2.3 Protein Molecules

Protein molecules can serve as marker units. The proteins comprise the following groups: enzymes (e.g. peroxidase, alkaline phosphotase, urease, beta-galactosidase, peptidases), fluorescing proteins (e.g. from GFP-family or phycobiliproteins (e.g. Phycoerythrin, Phycocyanin) availbale e.g. from Molecular Probes Inc.), antigen-binding proteins (e.g. antibodies, tetramers, affibodies (Nord et. al Nature Biotechnology, 1997, v. 15, p. 772-) or their components (e.g. Fab fragments), nucleic acid-binding proteins (e.g. transcription factors).

### 1.3.3.3.2.2.4 Nucleic Acid Chains

Nucleic acid chains, including oligonucleotides (modified and non-modified), can act as marker units. The length of these nucleic acid chains should fall preferably within the following ranges (number of nucleotide monomers in a chain): 10 to 20, 20 to 50, 50 to 100, 100 to 200, 200 to 500, 500 to 1000, 1000 to 5000, 5000 to 10000, 10000 to 100000. DNA, RNA, PNA molecules can be used. Nucleic acid chains can carry additional modifications, such as, for example, free amino groups, dyes and other signal-giving molecules, e.g. macromolecular substances, enzymes or nanocrystals (FIG. 7a, c). These macromolecular substances can be sterically demanding ligands (FIG. 7), discussed in the paragraph “sterical hindrace”. Modified nucleic acid chains are also commercially available, e.g. from MWG-Biotech.

Further examples of macromolecules or macromolecular complexes which can be used, according to the scope of the present invention, as a marker or marker units in the marker component are described in the U.S. Pat. No. 4,882,269, the U.S. Pat. No. 4,687,732, WO 8903849, the U.S. Pat. No. 6,017,707, the U.S. Pat. No. 6,627,469. Also other marker units can be used, like lectines, growth factors, hormones, reseptor molecules.

### 1.3.3.3.3 Core Component of the Marker

The core component has the function of connecting several structural elements of the modified nuc-macromolecules. For instance, the core component connects several marker units together. In a further embodiment, linker components can be bound to the core component (FIG. 5). The term “core-component” is functional and serves for illustration of possible structures of modified nuc-macromolecules. Different chemical structures that connect linker and marker-units can be called core-component. Examples for constituents of the core component will now be presented.

### 1.3.3.3.3.1 Constituents of the Core Component

In one embodiment, the core component consists of one or several low molecular compounds. They have the function of connecting the marker units together. A connection between the core component and the marker units can be covalent or affine. With covalent bonding, for instance, compounds with the general structural formula (F)ₘ—R—(H)ₙ can act as a precursor, where (F) and (H) are reactive groups and (R) a connecting component. The number of such groups and their assembly can vary considerably. Many examples are known to the expert in the field, e.g. connections from the group of crosslinkers (“Chemistry of protein conjugation and crosslinking” Shan S. Wong in 1993 CRC Press Inc). The structure is not limited. It is preferably water-soluble. For instance, parts (F) and (H) comprise independently the following groups: NH2 (amino), OH (hydroxy), SH (mercapto), COOH (carboxy), CHO (aldehyde), acrylic or maleimide. Water-soluble polymeres like PEG or polypetide chains or short aliphatic chains represent examples for (R).

In a further embodiment, the core component consists of a water-soluble polymer, wherein the said polymer can consist of the same or different monomers.

The following polymers and their derivates are examples of parts of the core component: polyamides (e.g. polypeptide like polyglutamin or polyglutamic acid) and their derivates, polyacrylic acid and its derivates, natural or synthetic polysaccharides (e.g. starch, hydroxy-ethyl-starch), dextran and its derivates (e.g. aminodextran, carboxydextran), dextrin, polyacrylamides and their derivates (e.g. N-(2-hydroxypropyl)-methacdylamide), polyvinyl alcohols and their derivates, nucleic acids, proteins. These polymers can be linear, globular, e.g. streptavidin or avidin, or can be branched, e.g. dendrimers. Also, cross-connected, soluble polymers, for instance, crosslinked polyacrylamides (crosslinker bisacrylamide in combination with polyacrylamide), are suitable.

Since the linker component as well as the marker component can contain water-soluble polymers, in one embodiment such a polymer can serve as a linker as well as a core component. In this case, one part of such a polymer can be considered as a linker, another part as core component.

In a preferred embodiment of the invention, linear polymers or polymers containing few branches are used as core components, for instance, polyamides (e.g., polypeptides), poly-acrylic acid, polysaccharides, dextran, poly(acrylamides), polyvinyl alcohols. The polymer can consist of identical or different monomers. Especialy in this embodiment, the linker component can have less than 50 chain atoms. Thus, linker lengths of approx. 5 to 10, 10 to 20 or 20 to 50 chain atoms can be sufficient to preserve the substrate properties of the modified nuc-macromolecules for enzymes. Such a core component of the marker fulfils the function of the linker component: it creates spatial distance between sterically demanding marker units and active centers of the respective enzymes.

The water-soluble polymers preferably have an average chain length of 20 to 1,000,000 chain atoms. For instance, an average chain length will be between 20 and 100, 100 and 500, 500 and 5000, 5000 and 100000, 100000 and 1000000 chain atoms.

In one embodiment, the polymer generally has a neutral form when dissolved in watery phase with a pH between 4 and 10 (e.g., dextran or polyacrylamide). In another embodiment, the polymer is charged if dissolved in a watery phase with a pH between 4 and 10. It can carry positive (e.g., polylysine) or negative charges (e.g., polyacrylic acid).

The coupling of marker units to a water-soluble polymer depends on the kind of the polymer. The reactive groups necessary for the coupling can already be present in the polymer (e.g., polylysine or polyacrylic acid) or can be introduced into the polymer in a separate step. For instance, many different variants for introducing reactive groups and chemical couplings are known for dextran. (Molteni L. Methods in Enzymology 1985, v. 112, 285, Rogovin A. Z. et al. J. Macromol Sci. 1972, A6, 569, Axen R. et al. Nature 1967, v. 214, 1302, Bethell G. S. et al. J. Biol. Chem. 1979, v. 254, 2572, Lahm O. et al. Carbohydrate Res. 1977, v. 58, 249, WO 93/01498, WO 98/22620, WO 00/07019).

The core component has in a favored application several coupling positions to which further elements can be bound, e.g. structural marker units or nuc-linker-components.

For instance, polylysine molecules have multiple free amino groups to which several dye molecules, biotin molecules, hapten molecules or nucleic acid chains can be coupled. Polylysines of different molecular mass are commercially available (e.g. 1000-2000 Da, 2000-10000 Da, 10000-50000 Da).

Nucleic acid strands constitute a further example of the core component and these chains have the following length ranges (number of nucleotide monomeres in a chain): 10 to 20, 20 to 50, 50 to 100, 100 to 200, 200 to 500, 500 to 1000, 1000 to 5000, 5000 to 10000. These nucleic acids act as a binding partner for sequence complementary marker-units (FIG. 6b).

In a further embodiment, the core component consists of a dendrimer, e.g. polypropylenimine or polyaminoamine. Examples of other dendrimers are known: Cientifica “Dendrimers”, in 2003, Technology white papers No. 6, Klajnert et al. Acta Biochimica Polonica, 2001, v. 48; p 199-, Manduchi et al. Physiol. Genomics 2002, v. 10; p 169-, Sharma et al. Electrophoresis. 2003, v. 24; p 2733-, Morgan et al. Curr Opin drug Discov Devel. 2002; v. 5 (6); p 966-73, Benters et al. Nucleic Acids Res. 2002, v. 30 (2): pE10, Nils et al. Theor Biol. 1997; v. 187 (2): p 273-84. Many dendrimers are commercially available (Genisphere, www.genisphere.com, Chimera Biotech GmbH).

Further combinations for the core component from the constituents described above are obvious to the specialist.

### 1.3.3.3.3.2 Coupling of the Marker Units

Marker units can be bound to the core component or to the linker component by a covalent bond, for example, via a crosslinker (Chemistry of protein conjugation and cross linking, S. Wang, 1993, ISBN 0-8493-5886-8, “Bioconjugation: protein coupling techniques for the biomedical sciences”, M. Aslam, 1996, ISBN 0-333-58375-2), or via an affine bond, for example, biotin-streptavidin connection or hybridizing of nucleic acid chains or antigen-antibody interaction (“Bioconjugation: protein coupling techniques for the biomedical sciences”, M. Aslam, in 1996, ISBN 0-333-58375-2).

In one embodiment, the coupling of the marker units to the core component is conducted already during the synthesis of the modified nuc-macromolecules.

In another embodiment, the chemically synthesized modified nuc-macromolecules comprise a marker component consisting only of a core component without marker units. The coupling of marker units to the core component is conducted after the modified nuc-macromolecules have been incorporated in the nucleic acid chain. Due to the large number of potential binding positions within the core component, the probability of the coupling of the marker units to the core component of incorporated nucleotides is therefore substantially larger in comparison to conventional nucleotide structures. The coupling chemistry depends in detail on the structure of the marker units and the structure of the core component.

Covalent coupling: In one embodiment, the connection between the marker units and the core component can be resistant, e.g. to temperatures up to 100° C., to pH ranges between 3 and 12, and/or resistant to hydrolytical enzymes (e.g., esterases). In another embodiment of the invention, the connection is cleavable under mild conditions.

Examples of the coupling of nucleic acids to dendrimers (this corresponds to a coupling of marker units to the core component) are described, e.g., in Shchepinov et al. Nucleic Acids Res. 1999; v. 27 (15):p 3035-41, Goh et al. Chem Commun (Camb). 2002; (24): p 2954.

### 1.3.3.3.3.3 Coupling Between Linker and Marker

The connection between the linker component and the marker depends on the respective structures of the marker units or the structure of the core component. In one embodiment, the linker component is bound directly to the signal-giving or signal-transmitting marker unit (FIG. 4a). The marker can consist of only one or several marker units.

In a further embodiment, one or several linker components are bound to the core component of the marker (FIG. 5d).

The connection between the linker component and the marker can be covalent as well as affine. Many examples are known to the specialist, e.g. “Bioconjugation: protein coupling techniques for the biomedical sciences”, M. Aslam, in 1996, ISBN 0-333-58375-2. “Chemistry of protein conjugation and crosslinking” Shan S. Wong in 1993 CRC Press Inc).

Covalent coupling: In one embodiment, the connection between the linker component and the marker can be resistant to, e.g., temperatures up to 130° C., pH ranges between 1 and 14, and/or resistant to hydrolytic enzymes (e.g. proteases, estarases). In another embodiment, the connection is cleavable under mild conditions.

According to some embodiments of this invention, macromolecular compounds used for the labeling of nucleotides comprise water-soluble polymers (see above). The linker of the nuc-modified macromolecules comprises water-soluble polymers too. A person skilled in the art should recognize that assignment of individual polymers to the linker or to the marker has a descriptive character.

### 1.3.3.3.4 Ratio of Nuc-Components in a Modified Nuc-Macromolecule

One modified nuc-macromolecule can comprise on average 1 to 2, 2 to 5, 5 to 10, 10 to 30, 30 to 100, 100 to 1000, 1000 to 10000, 10000 to 1000000, nuc-components. In particular, the use of nanostructures or nano- or microparticles allows for the coupling of a very large numbers of nuc-components on a such structure.

In one embodiment, all modified nuc-macromolecules have the same number of nuc-components per one modified nuc-macromolecule. For instance, a maximum of 4 biotin molecules can be bound per one strepavidin molecule; at a saturating concentration of nuc-linker components, a uniform population of modified nuc-macromolecules can be obtained.

In another embodiment, a modified nuc-macromolecule population has a defined average number of nuc-components per one modified nuc-macromolecule, however, in the population itself there is dispersion in the actual occupation of the modified nuc-macromolecules by nuc-components. In this case, the number of nuc-components per one modified nuc-macromolecule displays an average.

### 1.3.3.3.5 Ratio of Marker Units in a Modified Nuc-Macromolecule

The number of marker units in one modified nuc-macromolecule falls within the following ranges: 1 and 2, 2 and 5, 5 and 20, 20 and 50, 50 and 100, 100 and 500, 500 and 1000, 1000 and 10000, 10000 and 100000. In one embodiment, modified nuc-macromolecules have a definite number of signal-giving units per one marker. In another embodiment, a population of modified nuc-macromolecules has a varying number of marker units per one modified nuc-macromolecule and it does not need to have a definite value for every single modified nuc-macromolecule in a population.

In one embodiment, all the modified nuc-macromolecules have the same number of marker units per one modified nuc-macromolecule. For instance, a maximum of 4 biotin molecules can be bound per one strepavidin molecule, see “Avidin-Biotin-Technology”, Methods in Enzymology v. 184, 1990.

In another embodiment, a modified nuc-macromolecule population has a defined average number of marker units per one modified nuc-macromolecule, however, in the population itself, there is dispersion in the actual occupation of the modified nuc-macromolecules by marker units. An increasingly more uniform occupation of the modified nuc-macromolecules by marker units can be achieved by the use of saturating concentration during the synthesis of the marker component.

For instance, in cases where only qualitative detection is important, the exact number of marker units per one modified nuc-macromolecule has a subordinate role. In such cases the availability of a stable signal is important in itself.

To an expert in the field it should be evident that the said marker components have substantially greater molecule size and molecule measures, than the respective nuc-components themselves. Other examples of macromolecular marker components should readily suggest themselves to an expert in the field.

### 1.3.3.3.6 Substrate Properties of the Modified Nuc-Macromolecules

The nuc-component represents the basis for the substrate properties of the modified nuc-macromolecules. These properties can be modified by steric obstacle (see paragraph 1.3.19, sterically demanding ligand).

### 1.3.3.3.7 Function of the Markers

In one embodiment, the macromolecular marker component can have a signal-giving function. In another embodiment, it has a signal-transmitting function. In a further embodiment, it has a catalytic function. In a still further embodiment, it has an affine function. In a still further embodiment, the marker combines more than just one function, e.g. signal-giving as well as signal-transmitting function. Further combinations will be obvious.

In the case of signal-giving function, the marker component contains constituents coupled already during the chemical synthesis to modified nuc-macromolecules.

In the case of signal-transmitting function, the marker component contains constituents that allow for reaction with signal-giving molecules, so that they can develop their signaling properties after this reaction, see WO 2005 044836. For instance, a marker component consists of several biotin molecules, e.g. 100 Biotin molecules. After the incorporation of the modified nuc-macromolecules, a detection reaction can take place with modified streptavidin molecules. In another example, nucleic acid chains display the signal-transmitting function: after the incorporation of modified nuc-macromolecules, a hybridisation of uniform oligonucleotides with detectable units, e.g. fluorescent dyes (synthsized by MWG-Biotech), to the marker component can take place. In a further example, amino or mercapto groups have the signal-transmitting function, e.g. 50 amino groups per marker. After the incorporation of the modified nuc-macromolecules in the nucleic acid chain, a chemical modification with reactive components is conducted, e.g. with dyes, as described, for example, for incorporated allyl-amino-dUTP, Diehl et al. Nucleic Acid Research, in 2002, v. 30, No. 16 e79.

In another embodiment, the macromolecular marker component has a catalytic function (in the form of an enzyme or ribozyme). Different enzymes can be used, e.g. peroxidases or alkaline phosphatases. Due to the coupling of the particular enzyme to the nuc-component, after the incorporation of modified nuc-macromolecules to the nucleic acid strand, this enzyme is bonded covalently to the strand, also.

In a further embodiment, a macromolecular marker component has an affinity functionality to another molecule. Examples of such markers are streptavidin molecules, antibodies or nucleic acid chains.

In a still further embodiment, a marker has a function of a sterically demanding ligand and is itself such a ligand.

### 13.4 Low Molecular Marker

The state-of-the-art labeling of nucleotides, for instance, with one or two biotin molecules, one or two dye molecules, one or two hapten molecules (e.g., digoxigenin).

1.3.5 Conventionally modified nucleotide—a nucleotide with a linker (average length between 5 and 30 atoms) and a marker. A conventionally modified nucleotide usually carries a marker with low molecular weight, e.g. one dye molecule or one biotin molecule.

### 1.3.6 Enzymes (Polymerases)

In one embodiment, the modified nuc-macromolecules can be used as substrates for enzymes. Polymerases represent frequently used enzymes, which utilize nucleotides as substrates. They will be dealt with further as representative examples of other nucleotide-utilizing enzymes. One of the central abilities of polymerases consists in covalent coupling of nucleotide monomers to a polymer. Furthermore, the synthesis can be template-dependent (as for example DNA or RNA synthesis with DNA- or RNA-dependent polymerases) as well as independent of templates, e.g. terminal transferases (3 Sambrook “Molecular Cloning” 3. Ed. CSHL Press in 2001).

If RNA is used as a substrate (e.g., mRNA) in the sequencing reaction, commercially available RNA-dependent DNA polymerases can be used, e.g. AMV reverse transcriptase (Sigma), M-MLV reverse transcriptase (Sigma), HIV reverse transcriptase without RNAse activity. For certain applications, reverse transcriptases can be essentially free of RNAse activity (“Molecular cloning” in 1989, Ed. Maniatis, Cold Spring Harbor Laboratory), e.g. for use in mRNA labeling for hybridisation applications.

If DNA is used as a substrate (e.g. cDNA), all the following polymerases are suitable in principle: DNA-dependent DNA polymerases with or without 3′-5′ exonuclease activity (“DNA-Replication” in 1992 Ed. A. Kornberg, Freeman and company NY), e.g. modified T7-Polymerase of the type “Sequenase version 2” (Amersham Pharmacia Biotech), Klenow fragment of the DNA-Polymerase I with or without 3′-5′ exonuclease activity (Amersham Pharmacia Biotech), polymerase Beta of different origin (“Animal Cell DNA polymerases” in 1983, Fry M., CRC Press Inc, commercially available from Chimerx), thermostable polymerases such as, for example, Taq Polymerase, Vent-polymerase, Vent exo-minus, Deep Vent-polymerase, Deep Vent exo minus polymerase, Pfu-polymerase, Thermosequenase, Pwo-Polymerase (available for example from Promega GmbH, Amersham Biosciences (GE), Roche GmbH, New England Biolabs).

DNA-dependent RNA polymerases can also be used, e.g. E. coli RNA polymerase, T7 RNA polymerase, SP6 RNA polymerase.

Polymerases with 3′- or 5′-exonuclease activity can be used in certain applications (e.g. with real-time PCR).

In the following description, DNA-dependent DNA polymerases will be considered as examples of polymerases.

### 1.3.7 Cleavable Compound

A compound which is cleavable under mild conditions. This compound can represent a part in the linker and can be cleavable in one or several positions. It can be a chemically cleavable bond, such as, for example, disulfide, ester, acetal, thioester bonds (Short WO 9949082, Tcherkassov WO 02088382). It can also be a photo-chemically cleavable compound (Rothschild WO 9531429). It can also be an enzymatically cleavable compound (for instance, a peptide or polypeptide bond, Odedra WO 0192284), cleavable by peptidases, a poly- or oligo-saccharide bond, cleavable by disaccharidases, whereas the cleavage can be achieved by a specific enzyme between certain monomers of the cleavable bonds.

Several examples of cleavable compounds are known. The synthesis of such a compound is described, for instance, in (Tcherkassov WO 02088382, Metzker et al. Nucleic Acid Research 1994, v. 22, p. 4259-, Canard et al. Genes, 1994, v. 148, p. 1, Kwiatkowski U.S. Pat. No. 6,255,475, Kwiatkowski WO 0125247, Parce WO 0050642, Milton et al. WO 2004018493, Milton et al. 2004018497, WO2007053719). A cleavable compound can be a part of the linker or can form the connecting part of the linker to the nucleotide, or the connecting part of the linker component to the marker component, or the connection between marker units and the core component.

### 1.3.8 DNA

Deoxyribonucleic acid of different origin and different length (e.g. oligonucleotides, polynucleotides, plasmides, genomic DNA, cDNA, ssDNA, dsDNA)

### 1.3.9 RNA

Ribonucleic acid

1.3.10 dNTP

2′-deoxynucleoside triphosphate, as a substrate for DNA polymerases and reverse-transcriptases, e.g. dATP, dGTP, dUTP, dTTP, dCTP.

### 1.3.11 NTP

Ribonucleoside triphosphate, as a substrate for RNA polymerases, UTP, CTP, ATP, GTP.

### 1.3.12 NT

Abbreviation “NT” is used for the description of the length of a particular nucleic acid sequence, e.g. 1000 NT. In this case “NT” means nucleoside monophosphates.

The plural is formed by the addition of the suffix “-s”; “NT” means, for example, “one nucleotide”, “NTs” means “several nucleotides”.

### 1.3.13 NAC

Nucleic acid chain (NSK abbreviation stands for German “Nukleinsäurekette”), DNA or RNA.

### 1.3.14 Term “The Whole Sequence”

The whole sequence is the sum of all the sequences in one experiment; it can comprise originally one or several NACs. Also, the whole sequence can display parts or equivalents of another sequence or sequence populations (e.g., mRNA, cDNA, Plasmid DNA with insert, BAC, YAC) and can originate from one species or various species.

### 1.3.15 NACF

The nucleic acid chains fragment (NSKF abbreviation stands for German “Nukleinsäurekettenfragment”) (DNA or RNA) which corresponds to a part of the whole sequence, NACFs—the plural form—nucleic acid chain fragments. The sum of the NACFs forms an equivalent to the whole sequence. The NACFs can be, for instance, fragments of the whole sequence (DNA or RNA), which result after a fragmentation step.

### 1.3.16 Primer Binding Site (PBS)

A PBS is the part of the sequence in the NAC or NACF to which the primer binds.

### 13.17 Reference Sequence

A reference sequence is an already known sequence, divergences from which in the analysed sequence or sequences (e.g. whole sequence) have to be determined. Reference sequences can be found in databases, such as, for example, the NCBI database.

### 1.3.18 Tm

Melting temperature

### 1.3.19 Steric Obstacle, Sterically Demanding Group or Ligand

A group or a chemical structure, as a component of the modified nuc-macromolecule (as a functional part of modified nuc-macromolecule), which creates a space-demanding effect at a certain distance from the nucleotide. In a preferred embodiment, this chemical structure has the effect that a polymerase can incorporate only one complementary modified nuc-macromolecule into the primer and that an incorporation of further complementary modified nuc-macromolecules in direct proximity to the first incorporated modified nuc-macromolecule is inhibited.

### 1.3.19.1 Nature and Structure of the Sterically Demanding Ligand

Polymers (e.g., proteins, dendrimers) or supramolecular structures (e.g., nanoparticles or microparticles) with a compact three-dimensional (3D) structure are preferably used as macromolecular, sterically demanding ligands. Space-demanding properties are of importance for the description of macromolecular, sterically demanding ligands. Ideally, such a macromolecular ligand will occupy a certain volume, so that the presence of another macromolecular molecule in this volume is impossible or is very unlikely.

In a preferred embodiment of the invention, proteins are used as the example of sterically demanding ligands within the meaning of this application, e.g. streptavidin (SA), avidin, phycoerythrin (PE), green fluorescent protein (GFP), antibodies, bovine serum albumins (BSA) or their derivatives and modifications (e.g., alkylated, acetylated, or other forms of the proteins modified with other water-soluble polymers), or genetically modified proteins with other spectral properties or protein conjugates, as for example streptavidin-alkaline phosphatase, streptavidin-peroxidase, streptavidin-antibody, streptavidin-phycoerhytrin or entire complexes, as for example quantum dots with envelope formed by polyacrylic acid and streptavidin (available from Invitrogen).

In a further embodiment of the invention, dendrimers are used as the example of sterically demanding ligands within the meaning of this application (see paragraph “Marker”).

In a further embodiment of the invention, nanoparticles and microparticles are used as the example of sterically demanding ligands within the meaning of this application, e.g. paramagnetic particles, glass particles, plastic particles, (see paragraph “Marker”).

In a further embodiment of the invention, branched polymers are used as the example of sterically demanding ligands within the meaning of this application, e.g. dextrans, (see paragraph “Marker”).

### Weight/Dimension/Diameter

For the purpose of simplifying the classification of sterically demanding ligands, an indication of the weight/mass (e.g., for proteins) or of an average diameter (e.g., for nanostructures) will be used. These values serve as a rough measure for differentiating sterically demanding ligands according to their size. Accordingly, low-molecular, sterically demanding ligands (molecular weight less than 2 kDa) and macromolecular sterically demanding ligands (molecular weight larger than 2 kDa) are distinguished.

In a preferred embodiment, ligands having a molecular weight ranging from 2 to 1000 kDa are used. In particular, the mass of the steric obstacle can range between 2 and 10 kDa, 10 and 30 kDa, 30 and 100 kDa, 100 and 300 kDa, and 300 and 1000 kDa.

A further embodiment uses ligands with a diameter ranging between 1 and 3 nm, 3 and 10 nm, 10 and 30 nm, 30 and 100 nm, 100 and 300 nm, 300 nm and 1000 nm, and 1000 nm and 5000 nm.

In a further embodiment, ligands of low molecular weight are coupled to a scaffolding to form and act in combination (i.e. the ligands at themselves and the scaffolding) as a macromolecular sterically demanding ligand. Accordingly the number of the ligands with a low mass, coupled to a scaffolding, can range, for instance, between 2 and 200.

It should be obvious to person skilled in the art, that other features can also be used for classification of ligands, e.g., an indication of a chemical structure, the total charge, description of the surface properties, shape, or geometrical dimensions, or the volume etc. Additionally, it is assumed as known that the molecules or nanostructures can comprise different chemical groups.

1.3.19.2.1 Position of the Steric Obstacle within Modified Nuc-Macromolecule and its Coupling
In a preferred embodiment, a macromolecular sterically demanding ligand is coupled to the linker. The coupling position of the sterically demanding ligand can be inside or at the end of the linker. In this embodiment, the marker has an independent coupling position on the linker, which deviates from the coupling position of the sterically demanding ligand.
In a further preferred embodiment, the macromolecular sterically demanding ligand is coupled to the linker. The marker is coupled to this macromolecular sterically demanding ligand. In this embodiment, the ligand serves as a connecting part between the linker and the marker. The marker can contribute to the space-demanding effect of the sterically demanding ligand.
In a further preferred embodiment, the sterically demanding ligand is coupled to the marker. The marker can contribute to the space-demanding effect of the sterically demanding ligand.
In a further preferred embodiment, the sterically demanding ligand is a component of the marker. Both structures contribute to the space-demanding effect of the sterically demanding ligand. For instance, the sterically demanding ligand can serve as the core-component within the marker.
In a further preferred embodiment, the sterically demanding ligand has the marker function, i.e. the marker and the sterically demanding ligand are identical.

The sterically demanding group can be considered as a part of the linker or as a part of the marker. The point of view can depend, for instance, on whether the sterically demanding group does or does not have certain signal properties.

The number of the macromolecular sterically demanding ligands coupled to the modified nuc-macromolecule can range, for example, between 1 and 3, 3 and 5, 5 and 20, 20 and 50, and 50 and 1000. Accordingly, this number can be an exact or an average number.

The minimum distance between the nuc-component and the nearest sterically demanding ligand (“steric obstacle”) can range between 10 and 10000 chain atoms and preferably encloses following ranges: 10 to 15, 15 to 20, 20 to 25, 25 to 30, 30 to 35, to 40, 40 to 45, 45 to 50, 50 to 55, 55 to 60, 60 to 70, 70 to 80, 80 to 90, 90 to 100, 100 to 200, 200 to 1000, 1000 to 5000, and 5000 to 10000 chain atoms, or 10 to 15, 15 to 20, 20 to 25, 25 to 30, 30 to 35, 35 to 40, 40 to 45, 45 to 50, 50 to 55, 55 to 60, 60 to 70, 70 to 80, 80 to 90, 90 to 100, 100 to 200, 200 to 1000, 1000 to 5000, and 5000 to 10000 Angstroms (calculated on a stretched state of the molecule).

### General Meaning of the Distance Between the NT and Steric Obstacle:

The linker creates a distance between the enzymatically active nuc-component and the sterically demanding ligand. At sufficiently long distance, a polymerase can incorporate the nuc-component into a primer _{(N)} (the primer _{(N)} has no demanding ligand). Since the primer_{(N+1)} itself now carries a sterically demanding ligand on its 3′-OH end, this sterically demanding ligand prevents the incorporation of further modified nuc-macromolecules with sterically demanding ligands (see paragraph “Enzymatic properties of modified nuc-macromolecules”).

### 1.3.19.2.2 Coupling of a Macromolecular Sterically Demanding Ligand to the Linker

The linker-component and the macromolecular sterically demanding ligand can be connected similarly as described for the connection between the linker and the marker. It can be a covalent or affine coupling. Many examples are known to a person skilled in the art (e.g., “Bioconjugation: protein coupling techniques for the biomedical sciences”, M. Aslam, in 1996, ISBN 0-333-58375-2. “Chemistry of protein conjugation and crosslinking”, Shan S. Wong in 1993 CRC Press Inc). Covalent bond: in one embodiment, the connection between the linker-component and the marker can be resistant towards temperatures up to 130° C. or pH ranges between 1 and 14, and/or resistant against hydrolytic enzymes (e.g., proteases, esterases). In another embodiment, the bond between the nuc-component and linker is cleavable under mild conditions.

Several embodiments, which describe structures in which a macromolecular sterically demanding ligand is coupled to the linker and the marker on its part is coupled to the ligand, or in which the ligand serves as a core-component of the marker, are included under “Examples”.

### 1.3.19.2.3 Substrate Properties of the Modified Nuc-Macromolecules:

Sterically demanding ligands can modify the properties of nuc-components vis-à-vis polymerases.

For sake of descriptiveness, the substrate properties of modified nuc-macromolecules vis-à-vis a primer_{(N)}-template-polymerase complex (the primer_{(N)} does not comprise a sterically demanding ligand) can be explained in that the complementary nuc-component of a modified nuc-macromolecule has a sufficient working distance and the steric obstacle does not prevent the polymerase from incorporating this nuc-component into the primer_{(N)}-template complex. (The idea of the inventors does not claim to be complete and is intended to only schematically describe the basic principles of space-demanding properties).

After the incorporation of the first modified nuc-macromolecule (Primer_{(N+1)}) the situation changes: The steric obstacle is occupying the space, so that it cannot be taken by another big structure (e.g., similarly large or even larger sterically demanding ligand). The effectively occupied space is determined by the volume of the molecule itself and influences that arise in the solution (e.g., solvent envelopes, which contribute to a hydrodynamic diameter) so that this space can be larger than the actual volume of the molecular structure. Due to the coupling of the steric obstacle within the modified nuc-macromolecule, the sterically demanding ligand can be placed near the 3′-OH group.

The substrate properties (an ability to incorporate the next complementary nucleotide/the next complementary nuc-component) of the complex, consisting of template, the extended Primer_{(N+1)}, the polymerase and the steric obstacle bonded to the terminal nucleotide, can be summarized as follows:
Low-molecular weight nucleotides and their derivatives still have access to the active center of the polymerases (e.g., other complementary natural nucleotides and their low-molecular derivatives, e.g., nucleotides modified with a dye, e.g., dCTP-Cy) and can be incorporated.
complementary nuc-components of the modified nuc-macromolecules do not have access to the active center of the polymerase, because the macromolecular sterically demanding ligand of the modified nuc-macromolecule cannot get near the polymerase and the working distance of the nuc-component is limited by the linker length.

With increasing distance from the sterically demanding ligand, e.g., after repeated incorporations of natural nucleotides into the primer after the modified nuc-macromolecule (Primer_{(N+X)}), the effect of the steric obstacle decreases, so that further modified nuc-macromolecule can be incorporated once more.

After the steric obstacle has cleaved-off from the incorporated nuc-component, the primer-template-polymerase complex (Primer_{(N+1)}) loses the space-demanding ligand, so that the accessibility of another nuc-component of the modified nuc-macromolecule is restored.

### 1.3.20 Compositions

As component of a kit, the composition for carrying out one or more method steps may be a solution containing one or several substances or also a dry mixture, which must be added to a solution prior to the method step.

### 1.3.21 Solid Phase/Stationary Phase/Reaction Surface

In preferred embodiments of the invention, the nucleic acid chains participating in the reaction are attached to a solid phase. The attachment may be covalent or affine. In this connection, the terms “solid phase”, “stationary phase” and, “reaction surface” will be used as synonyms, unless another meaning is pointed out.

### 2. DESCRIPTION OF THE INVENTION

The invention includes the following aspects:

Aspect 1: Nucleotide analogs (the modified nuc-macromolecules) comprising the following components: at least one nucleotide component (nuc-component), at least one macromolecular sterically demanding ligand, at least one marker, at least one linker.

Aspect 2: Nucleotide analogs (the modified nuc-macromolecules) comprising the following components: at least one nucleotide component (nuc-component), at least one macromolecular sterically demanding ligand, at least one marker, at least one linker wherein the linker that is coupled to the nucleotide component is cleavable.

Aspect 3: A reaction mixture comprising at least one of the nucleotide analogs according to aspect 1 or 2.

Aspect 4: A composition comprising at least one of the nucleotide analogs according to aspect 1 or 2:The ratio between the weight percentage of the nucleotide analog and the weight of the composition comprises the following ranges: 1:1000000 to 1:100000, 1:100000 to 1:10000, 1:10000 to 1:1000, 1:1000 to 1:100, 1:100 to 1:10, 1:10 to 1.

Aspect 5: A nucleic acid chain or a mixture of nucleic acid chains comprising at least one of the nucleotide analogs according to aspect 1 or 2 as a monomer of the nucleic acid chain, wherein the nucleic acid chains can be in a solution or fixed to a solid phase.

Aspect 6: A nucleic acid chain or a mixture of nucleic acid chains according to aspect 5, wherein these nucleic acid chains have a primer function.

Aspect 7: Method for enzymatic synthesis of the nucleic acid chains, wherein the nucleotide analogs according to aspect 1 or 2 are used.

Aspect 8: A method for the synthesis of nucleic acid chains comprising the following steps:
Preparation of extendable template-primer complexes
Incubation of these complexes in a reaction solution, which comprises one or several types of polymerases and at least one type of the modified nuc-macromolecules according to aspect 2, under conditions which allow for primer extension by a modified nuc-macromolecule, wherein the modified nuc-macromolecule is modified in such a way that its incorporation causes further enzymatic reaction to stop

Aspect 9: A kit for carrying out enzymatic synthesis of nucleic acid chains comprising the following elements:
One or several kinds of polymerases
At least one of the nucleotide analogs, according to aspect 1 or 2

Aspect 10: A Kit for sequencing nucleic acid chains comprising the following elements:
One or several kinds of polymerases
At least one of the nucleotide analogs according to aspect 2

Aspect 11 A method for sequencing of nucleic acid chains comprising the following steps:
a) Preparation of at least one population of extendable nucleic acid chain-primer complexes (NAC-primer complexes),
b) Incubation of at least one type of the modified nuc-macromolecule according to aspect 2 together with at least one type of polymerase with the NAC primer complexes prepared in step (a) under conditions which allow for the incorporation of complementary modified nuc-macromolecules, each type of modified nuc-macromolecule having a distinctive label,
c) Removal of the unincorporated modified nuc-macromolecules from the NAC primer complexes,
d) Detection of the signals from the modified nuc-macromolecules which have been incorporated in the NAC primer complexes,
e) Removal of the linker component and the marker component and the macromolecular sterically demanding ligand from the modified nuc-macromolecules which have been incorporated in the NAC primer complexes,
f) Washing of the NAC-primer complexes,
if necessary, repetition of the steps (b) to (f).

A further aspect 12 of the invention relates to a method according to aspect 11, wherein the nucleic acid chains are attached to a solid phase in random order, and at least a part of this NAC-primer complex is individually optically addressable

A further aspect 13 of the invention relates to a method according to aspect 11 for the parallel sequence analysis of nucleic acid sequences (nucleic acid chains, NACs), in which
fragments (NACFs) of single-stranded NACs with a length of approximately 50 to 1000 nucleotides that may represent overlapping partial sequences of the whole sequence are produced,
the NACFs are bonded to a reaction surface in a random order using a uniform primer or several different primers in the form of NACF-primer complexes, wherein the density of NACF-primer complexes bonded to the surface allows for an optical detection of signals from single incorporated modified nuc-macromolecules,
a cyclical synthesis reaction of the complementary strand of the NACFs is performed using one or more polymerases by
   a) adding, to the NACF primer complexes bonded to the surface, a solution containing one or more polymerases and one to four modified nuc-macromolecules according to aspect 2 that have a marker component labeled with fluorescent elements, wherein the fluorescent elements, which each are located on the marker component when at least two modified nuc-macromolecules are used simultaneously, are chosen in such a manner that the nuc-macromolecules used can be distinguished from one another by measuring different fluorescent signals, the modified nuc-macromolecules being structurally modified in such a manner that the polymerase is not capable of incorporating another nuc-macromolecule in the same strand after such a modified nuc-macromolecule has been incorporated in a growing complementary strand, the linker component and marker component and macromolecular sterically demanding ligand being removable,
   b) incubating the stationary phase obtained in step a) under conditions suitable for extending the complementary strands, the complementary strands each being extended by one modified nuc-macromolecule,
   c) washing the stationary phase obtained in step b) under conditions suitable for removing modified nuc-macromolecules that are not incorporated in a complementary strand,
   d) detecting the single modified nuc-macromolecules incorporated in complementary strands by measuring the characteristic signal of the respective fluorescent elements, the relative position of the individual fluorescent signals on the reaction surface being determined at the same time,
   e) cleaving-off the linker component and marker component and the macromolecular sterically demanding ligand from the modified nuc-components added to the complementary strand in order to produce unlabeled NACFs,
   f) washing the stationary phase obtained in step e) under conditions suitable for the removal of the marker component,
   repeating steps a) to f), several times if necessary,
the relative position of individual NACF-primer complexes on the reaction surface and the sequence of these NACFs being determined by specific assignment of the fluorescent signals that were detected in the respective positions in step d) during successive cycles to the modified nuc-macromolecules.
A further aspect 14 of the invention relates to a method according to aspect 13, characterized in that steps a) to f) of the cyclical synthesis reaction are repeated several times, only one type of modified nuc-macromolecule being used in each cycle.
A further aspect 15 of the invention relates to a method according to aspect 13 characterized in that steps a) to f) of the cyclical synthesis reaction are repeated several times, two types of differently labeled modified nuc-macromolecules being used in each cycle.
A further aspect 16 of the invention relates to a method according to aspect 13 characterized in that steps a) to f) of the cyclical synthesis reaction are repeated several times, four types of differently labeled modified nuc-macromolecules being used in each cycle.

Aspect 17: A kit for sequencing method of nucleic acid chains according to one of the aspects 8 or 11 to 15 comprising the following elements:
One or several kinds of polymerases,
At least one of the nucleotide analogs according to aspect 2,
Solutions for performing cyclic sequencing steps.

Aspect 18: A kit for sequencing nucleic acid chains according to the method according to one of the aspects 8 or 11 to 15 comprising one or several of the following compositions, provided as a solution in concentrated or in diluted form or also as a mixture of dry substances, from the following list:
One or several kinds of the polymerases,
At least one of the nucleotide analogs, according to aspect 2,
Solutions for performing cyclic sequencing steps,
Composition for incorporation reaction/extension reaction,
Composition for washing the solid phase after the incorporation reaction,
Composition for optical detection of the signals on the solid phase,
Composition for cleaving-off of the marker and the sterically demanding macromolecular ligand,
Composition for washing the solid phase after the cleaving-off of the marker and the sterically demanding macromolecular ligand,
Composition for blockade of the linker residue,
Composition for washing the solid phase after the blockade of the linker residue,
Composition for binding signal-giving marker units to the marker,
Composition with signal-giving marker units.

Aspect 19: A kit for sequencing nucleic acid chains according to aspect 18 which furthermore comprises one or several elements from the following list:
Composition with unmodified nucleotides (dNTPs or NTPs),
Composition with irreversible terminators (ddNTPs),
Composition with terminal transferase,
Composition with a buffer for transferase reaction,
Composition with a ligase,
Composition of oligonucleotides which, as a uniform primer-binding site, can be ligated to the nucleic acid,
Composition with a buffer for ligase reaction,
Solid phase and reagents for preparing nucleic acid chains for the sequencing,
Solid phase and reagents for preparing polymerase for the sequencing,
Device and reagents for preparing nucleotide analogs according to aspect 2 for the sequencing,
Composition with blocking reagents for suppression of unspecific adsorption of labeled molecules,
Solid phase for performing cyclic incorporation reactions.

Aspect 20: A kit for sequencing method of nucleic acid chains according to one of the aspects 9, 10, 17, 18 or 19 which comprises one or more polymerases from the following list:
Reverse transcriptases: M-MLV, RSV, AMV, RAV, MAV, HIV
DNA polymerases: Klenow fragment DNA Polymerase, Klenow fragment exo-minus DNA Polymerase, T7 DNA polymerase, Sequenase 2, vent DNA polymerase, vent exo-minus DNA polymerase, Deep Vent DNA polymerase, Deep Vent exo-minus DNA polymerase, Taq DNA polymerase, Tli DNA polymerase, Pwo DNA polymerase, ThermoSequenase DNA polymerase, Pfu DNA polymerase.

Aspect 21: A kit for sequencing nucleic acid chains according to one of the aspects 9, 10, 17, 18 or 19, wherein the components of the compositions are already mixed or are provided as substances in separated form.

Aspect 22: A kit for sequencing nucleic acid chains according to one of the aspects 9, 10, 17, 18 or 19 which comprises one or more solid phases for the performance of cyclic sequencing steps from the following list:
A planar, transparent solid phase,
A planar, transparent solid phase which is provided as a component of a flow-cell or a chip,
A solid phase in form of nano- or microbeads,
A solid phase in form of nano- or microbeads which are paramagnetic,
Solid phase prepared according to patent application DE 101 49 786,
Solid phase prepared according to patent application DE 10 2004 025 744.

Aspect 23: A method for the synthesis of nucleic acid chains which comprises the following steps:
a) Preparation of extendable primer-template complexes,
b) Incorporation reaction: Incubation of these complexes in a reaction solution containing one or more kinds of polymerase and of at least one type of the modified nuc-macromolecule according to aspect 2 under conditions which allow a primer extension by one modified nuc-macromolecule, wherein the modified nuc-macromolecule is modified in such a way that its incorporation causes further enzymatic synthesis to stop,
c) Incubation of the primer-template complexes under conditions which allow for separation of the said primer with incorporated nucleotide analogs from the template,
d) If necessary, repetition of the steps (b) to (c),
e) Application of the obtained labeled primer to a separation medium or in a separation process,
f) Optionally, identification of the type of the nucleotide analog incorporated.

The cyclic steps can be repeated several times, for instance, 2 to 10 times, 10 to 20 times, 20 to 100 times or 100 to 500 times. The identification of the incorporated nucleotide analogs is accomplished by means of the marker.

Aspect 24: A method for the synthesis of nucleic acid chains comprising the following steps:
a) Preparation of extendable primer-template complexes having addressable positions,
b) Incorporation reaction: Incubation of these complexes in a reaction solution, containing one or more kinds of polymerase and of at least one type of the modified nuc-macromolecules according to aspect 2 under conditions which allow a primer extension by one modified nuc-macromolecule, wherein the modified nuc-macromolecule is modified in such a way that its incorporation causes further enzymatic synthesis to stop,
c) Optionally, use of purification steps for template-primer complexes.
d) optionally, identification of the type of incorporated nucleotide analog by detecting marker characteristics, wherein a positional assignment of signals to particular primer-template complexes may be done.
e) Removal of the terminating macromolecular sterically demanding ligand and optionally the marker,
f) Optionally, use of purification steps for template-primer complexes,
g) if necessary, repetition of the steps (b) to (f) and subsequent analysis of the signals identified from incorporated nucleotide analogs.

The cyclic steps can be repeated several times, for instance, 2 to 10 times, 10 to 20 times, 20 to 100 times or 100 to 500 times. The identification of the incorporated nucleotide analogs is accomplished by means of the marker.

Aspect 25 of the invention relates to nucleotide analogs (modified nuc-macromolecules) with the composition according to aspect 1 or 2 comprising the following arrangments of components:

(Nuc-Linker 1)ₙ-(Ligand)ₖ-(Marker)ₘ
(Nuc-Linker 1)ₙ-(Ligand-Linker 3)ₖ-(Marker)ₘ
(Nuc-Linker 1)ₙ-(Ligand)ₖ-(Linker 3-Marker)ₘ
(Nuc-Linker 1)ₙ-(Marker)ₘ-(Ligand)ₖ

### (Nuc-Linker 1-Ligand)ₙ-(Marker)ₘ

(Ligand-Linker 2-Nuc-Linker 1)ₙ-(Marker)ₘ
(Nuc-Linker 1)ₙ-(Marker/Ligand)ₘ

### (Nuk-Linker 1-Ligand)ₙ-(Marker)ₘ-(Linker 1-Nuk)ₙ

wherein:
Nuc—is a nuc-component
Linker—is a linker component, wherein linker 1 or linker 2 or linker 3 can have identical or different structures
Marker—is a marker component
Ligand—is a macromolecular sterically demanding ligand
Marker/ligand—is a structure that has properties both of a marker and of a macromolecular, sterically demanding ligand
n—is a positive integer from 1 to 100000
m—is a positive integer from 1 to 1000
k—is a positive integer from 1 to 1000

In one embodiment, the structure comprises the following distribution within the molecule: (n)≧(m)≧(k), wherein individual numbers can be varied independently of one another. In a further embodiment, the structure comprises the following distribution: (n)>(m)>(k), wherein individual figures can be varied independently of one another. In a further embodiment, the structure comprises the following distribution: (n)=<(m)>(k), wherein individual figures can be varied independently of one another.

A further aspect 26 of the invention relates to macromolecular compounds according to aspect 1, 2 or 25, wherein the nuc-component comprises the following structures (FIG. 3A), wherein:
Base is selected independently from the group of adenine, or 7-deazaadenine, or guanine, or 7-deazaguanine, or thymine, or cytosine, or uracil, or their modifications, wherein (L) is the linkage between the nuc-component and the linker component (coupling unit L) and X is the coupling position of the coupling unit (L) to the base.
R₁— is H
R₂— is selected independently from the group of H, OH, halogen, NH₂, SH or protected OH group
R₃— is selected independently from the group of H, OH, halogen, PO₃, SH, N₃, NH₂, O—R₃₋₁, P(O)ₘ—R₃₋₁ ((m) is 1 or 2), NH—R₃₋₁, S—R₃₋₁, Si—R₃₋₁ wherein R₃₋₁ is a chemically, photochemically or enzymatically cleavable group or comprises one of the following modifications: —CO—Y, —CH₂—N₃, —CO—O—Y, —CO—S—Y, —CO—NH—Y, —CH₂—CH═CH₂, wherein Y is an alkyl, for instance (CH₂)ₙ—CH₃ wherein n is a number between 0 and 4, or a substituted alkyl, for instance with halogen, hydroxy group, amino group, carboxy group.
R₄— is H or OH
R₅— is selected independently from the group of OH, or a protected OH group, or a monophosphate group, or a diphosphate group, or a triphosphate group, or is an alpha thiotriphosphate group.

A further aspect 27 of the invention relates to nucleotide analogs according to aspect 1, 2 or 25, wherein the nuc-component comprises the following structures (FIG. 3B),
Wherein:
Base is selected independently from the group of adenine, or 7-deazaadenine, or guanine, or 7-deazaguanine, or thymine, or cytosine, or uracil, or their modifications capable of enzymatic reactions.
R₁— is H
R₂— is selected independently from the group of H, OH, halogen, NH₂, SH or protected OH group
R₃— is selected independently from the group of O—R₃₋₂-L, P(O)ₘ—R₃₋₂-L and (m) is 1 or 2, NH—R₃₋₂-L, S—R₃₋₂-L, Si—R₃₋₂-L, wherein R₃₋₂ is the coupling position of the linker to the nucleotide and L is the coupling unit (L) of the linker.
R₄— is H or OH
R₅— is selected independently from the group of OH, or a protected OH group, or a monophosphate group, or a diphosphate group, or a triphosphate group, or is an alpha-thiotriphosphate group.

A further aspect 28 of the invention relates to nucleotide analogs according to aspect 1, 2 or 25, wherein the nuc-component comprises the following structures (FIG. 3B),
Wherein:
Base is selected independently from the group of adenine, or 7-deazaadenine, or guanine, or 7-deazaguanine, or thymine, or cytosine, or uracil, or their modifications capable of enzymatic reactions.
R₁— is H
R₂— is selected independently from the group of H, OH, halogen, NH₂, SH or protected OH group
R₃— is selected independently from the group of H, OH, halogen, PO₃, SH, NH₂, O—R₃₋₁, P(O)ₘ—R₃₋₁ ((m) is 1 or 2), NH—R₃₋₁, S—R₃₋₁, Si—R₃₋₁ wherein R₃₋₁ is a chemically, photochemically or enzymatically cleavable group.
R₄— is H or OH
R₅— is selected independently from the group of O—R₅₋₁-L, or P—(O)₃—R₅₋₁-L (modified monophosphate group), or P—(O)₃—P—(O)₃—R₅₋₁-L (modified diphosphate group) or P—(O)₃—P—(O)₃—P—(O)₃—R₅₋₁-L (modified triphosphate group), wherein R₅₋₁ is the coupling position of the coupling unit (L) to the nuc-component and coupling unit (L) is a linkage between nuc-component and linker-component.

A further aspect 29 of the invention relates to nucleotide analogs according to aspects 26 to 28, wherein the coupling unit (L) of the linker comprises the following structural elements:
R₆—NH—R₇, R₆—O—R₇, R₆—S—R₇, R₆-SS-R₇, R₆—CO—NH—R₇, R₆—NH—CO—R₇, R₆—CO—O—R₇, R₆—O—CO—R₇, R₆—CO—S—R₇, R₆—S—CO—R₇, R₆—P(O)₂—R₇, R₆—Si—R₇, R₆—(CH₂)ₙ—R₇, R₆—(CH₂)ₙ—R₇, R₆-A-(CH₂)ₙ—R₇, R₆—(CH₂)ₙ—B—R₇, R₆—(CH═CH—)ₙ—R₇, R₆-(A-CH═CH—)ₙ—R₇, R₆—(CH═CH—B—)ₙ—R₇, R₆—(CH═CH—CH₂—B—)ₙ—R₇, R₆-A-CH═CH—(CH₂—)ₙ—R₇, R₆—(—CH═CH—CH₂)ₙ—B—R₇, R₆-(A-C≡C—)ₙ—R₇, R₆-(A-C≡C—)ₙ—R₇, R₆-(A-C≡C—CH₂)ₙ—R₇, R₆—(C≡C—B—)ₙ—R₇, R₆—(C≡C—CH₂—B—)ₙ—R₇, R₆-A-C≡C—(CH₂—)ₙ—R₇, R₆—(—C≡C—CH₂)ₙ—B—R₇, R₆—(—C≡C—CH₂—CH₂)ₙ—B—R₇
wherein R₆ is the nuc-component, R₇ is the rest of the linker, and A and B comprise independently the following structural elements: —NH—, —O—, —S—, -SS-, —CO—NH—, —NH—CO—, —CO—O—, —O—CO—, —CO—S—, —S—CO—, a photolabile group, —P(O)₂—, —Si—, —(CH₂)ₙ—, wherein (n) ranges from 1 to 5,

A further aspect 30 of the invention relates to nucleotide analogs according to aspects 25 to 28, wherein the linker-component comprises a water-soluble polymer.

A further aspect 31 of the invention relates to macromolecular compounds according to aspect 30, wherein the linker-component comprises water-soluble polymers selected independently from the following group:
polyethylene glycol (PEG), polysaccharides, dextran, polyamides, polypeptides, polyphosphates, polyacetates, polyalkyleneglycoles, copolymers from ethyleneglycol and propyleneglycol, polyolefinic alcohols, polyvinylpyrrolidones, poly(hydroxyalkylmethacrylamides), polyhydroxyalkylmethacrylates, poly(x-hydroxy) acids, polyacrylic acid, polyacrylamide, polyvinylalcohol.

A further aspect 32 of the invention relates to nucleotide analogs according to one of the aspects 1, 2 25 to 31, wherein the average length of a linker component ranges between 50 to 100, 100 to 200, 200 to 500, 500 to 1000, 1000 to 2000, 2000 to 10000, 10000 to 100000, 100000 to 500000 atoms (chain atoms).

A further aspect 33 of the invention relates to nucleotide analogs according to one of the aspects 1, 2 25 to 32, wherein a marker component having a signal-giving function, a signal-transmitting function, catalytic function or affine function, or function of a macromolecular sterically demanding ligand

A further aspect 34 of the invention relates to nucleotide analogs according to one of the aspects 25 or 33, wherein a structural marker unit independently comprises one of the following structural elements: biotin, hapten, radioactive isotope, rare-earth atom, dye, fluorescent dye.

A further aspect 35 of the invention relates to nucleotide analogs according to one of the aspects 25 to 33, wherein a structural marker unit independently comprises one of the following elements: nanocrystals or their modifications, proteins or their modifications, nucleic acids or their modifications, particles or their modifications.

A further aspect 36 of the invention relates to macromolecular compounds according to aspect 35, wherein a structural marker unit comprises one of the following proteins:
enzymes or their conjugates or modifications,
antibodies or their conjugates or modifications,
streptavidin or its conjugates or modifications,
avidin or its conjugates or modifications

Aspect 37 of the invention relates to nucleotide analogs according to one of the aspects 1, 2, or 25 to 36, wherein a macromolecular sterically demanding ligand comprises the following structures: proteins, dendrimers, nanoparticles, microparticles or their modifications.

In special embodiments, the methods presented above can be used for the identification of nucleic acids or for the identification of nucleic acid composition, i.e. the nucleotide sequence of the nucleic acids

According to a special embodiment of the invention, it is possible to carry out the methods by repeating the steps of the incorporation reaction (b) using:
a) only one labeled modified nuc-macromolecule in each step,
b) two differently labeled modified nuc-macromolecules in each step,
c) four differently labeled modified nuc-macromolecules in each step.

If combinations of several modified nuc-macromolecules are used, each kind of modified nuc-macromolecules has its own specific label.

The division of methods into individual steps is functional and should illustrate the structure of the method. Each of the above steps can be performed within a method as an individual, independent step or can be divided into further steps.

### Template

The template can be DNA or RNA molecules. It can be a uniform population of nucleic acid molecules or can comprise a mixture of nucleic acids with different sequences. Preferably, the template is provided in single-stranded form. If a double-stranded template is present, template-primer complexes can be formed by denaturation of the template and the subsequent hybridization of the primer.

The template comprises the following nucleic acids, among others: defined amplificates (e.g., PCR products), cDNA, fragments of the genomic DNA or RNA (also products of the amplification reactions), mRNA. Viral, bacterial or eukaryotic nucleic acid chains can be used.

In one embodiment, the template is dissolved in a solution. In another embodiment, the template is attached to a solid phase (via covalent, affine or another kind of the coupling).

Accordingly, the attachment to the solid phase can be in a defined order, for example with microarray or by using of beads with special coding (“Microarray biochip technology” 2000 M. Schena Eaton Publishing, “DNA Microarrays” 1999 M. Schena Oxford University Press, Fodor et al. Science 1991 v. 285 p. 767, Timofeev et al. Nucleic Acid Research (NAR) 1996, v.24 p. 3142, Ghosh et al. NAR 1987 v. 15 p. 5353, Gingeras et al. NAR 1987 v. 15 p. 5373, Maskos et al. NAR 1992 v. 20 p. 1679). The attachment can be also in a random order, for example in WO 02088382, DE 10 2004 025 696, DE 101 20 798, DE 102 14 395.

Primer or Oligonucleotide with the Primer-Function:

The primer can be an oligodeoxinucleotide or an oligoribonucleotide.

In one embodiment of the invention, uniform primers are used. In another embodiment, primers with different sequences are used.

The composition and the length of the primers are not limited. A primer can have also other functions besides the start-function, e.g., to create a connection to the reaction surface. A primer can comprise segments of nucleic acids that are not complementary to the template and serve, for instance, to bond the primer to a solid phase.

The length and compositions of the primers should be adapted to the primer binding sites in the templates such that the primer makes it possible to start a sequencing reaction with a respective polymerase. In one embodiment of the invention, the primer is completely complementary to the corresponding primer binding site. In another embodiment of the invention, the primer has at least one non-complementary position to the primer binding site within the template.

If different primer binding sites are used, for instance, such as those naturally occurring in the original complete sequence, then sequence-specific primers are used for the respective primer binding site. A uniform primer can be used for a uniform primer binding site, such as a primer binding site coupled to the nucleic acid chains fragments via ligation.

Preferably, the length of the primer ranges between 6 and 100 NTs, more preferably between 10 and 50 NTs. The Primer can comprise a functional group which serves for the immobilization of the primer or primer-template, for instance, a biotin group is such a functional group. The synthesis of such a primer can be accomplished, e.g., with the DNA synthesizer 380A made by Applied Biosystems or be produced as custom synthesis by a commercial provider, e.g., MWG-Biotech GmbH, Germany).

Oligonucleotides can be fixed with different techniques or can be synthesized directly on the surface, for instance, as described in (McGall et al. U.S. Pat. No. 5,412,087, Barrett et al. U.S. Pat. No. 5,482,867, Mirzabekov et al. U.S. Pat. No. 5,981,734, “Microarray biochip technology” 2000M. Schena Eaton Publishing, “DNA Microarrays” 1999 M. Schena Oxford University Press, Fodor et al. Science 1991 v. 285 p. 767, Timofeev et al. Nucleic Acid Research (NAR) 1996, v. 24 p. 3142, Ghosh et al. NAR 1987 v. 15 p. 5353, Gingeras et al. NAR 1987 v. 15 p. 5373, Maskos et al. NAR 1992 v. 20 p. 1679).

The primer can be bonded to the surface, for instance, in a density ranging between 10 to 100 per 100 μm², 100 to 10,000 per 100 μm² or 10,000 to 1,000,000 per 100 μm².

The primer or primer mixture is incubated with the template under hybridization conditions that allow it to selectively bind to the respective primer binding sites within template. The optimization of the hybridization conditions depends on the precise structure of the primer binding site and that of primer and can be calculated by the method of Rychlik et al. NAR 1990 v. 18 page 6409. In the following, these hybridization conditions will be called standardized hybridization conditions.

### The Reaction Mixtures for an Incorporation Step/Extension Step can Comprise the Following Components:

an aqueous solution,
optional presence of suitable buffer substances (e.g., Tris buffer, phosphate buffer, acetate buffer, HEPES buffer, MOPS buffer, borate buffer); the concentration of the substances preferably ranges between 10 mmol/l and 200 mmol/l, the pH-Value of the solution preferably ranges between 5 and 10.
optional presence of monovalent metal ions (Na+, K+, Li+)
optional presence of divalent metal ions (e.g., Mg2+, Mn2+ or Co2+)
optional presence of organic solvent (e.g., DMF, DMSO) or other organic substances usually used for incorporation reactions, like glycerin, Tween 20, (for further information, see manufacturer's recommendations for individual polymerases).
optional presence of unmodified nucleotides (e.g., dCTP, dATP, dGTP, dTTP, dUTP, ATP, CTP, GTP, UTP) or conventionally modified nucleotides (e.g., biotin-16-dUTP, Cy3 dCTP or digoxigenin-dUTP)
optional presence of one or several kinds of polymerase which can incorporate a nucleotide to the primer in the primer-template complex in a template-dependent enzymatic reaction. The polymerases can have processive or distributive properties during the synthesis.
Presence of modified nuc-macromolecules, wherein
   only one labeled modified nuc-macromolecule is present,
   only two differently labeled, modified nuc-macromolecules are present,
   four differently labeled modified nuc-macromolecules are present.
optional presence of one or several other proteins which can bind to one of the reaction components, e.g., single-strand binding protein, e.g., elongation factors.
optional presence of marker units or marker components.

Temperature conditions for individual steps of the method according to the invention can be the same or can differ. They preferably range between 10° C. and 95° C.

### Purification Steps

These steps represent an optional purification of the template-primer complexes with incorporated modified nuc-macromolecules from the freely modified nuc-macromolecules in the solution. This purification can occur, for instance, via washing of the said extended template-primer complexes bonded to a solid phase. The washing can be accomplished, for instance, with a buffer solution.

The modified nucleotide analogs (modified nuc-macromolecules) used in the step (b) in the abovementioned methods are modified nuc-macromolecules comprising at least one macromolecular, sterically demanding ligand which, after the incorporation of a modified nuc-macromolecule, stops or significantly impedes the further enzymatic incorporation of such modified nuc-macromolecules. The efficiency of the prevention of the further progress of the incorporation reaction is preferably higher than 70%.

Reversible terminators with termination efficiencies ranging between 80 to 100% and 90 to 100% are preferred for sequencing methods. Particularly preferable are reversible terminators with termination efficiencies in the ranges between 95 to 100%, 97 to 100%, and 99 to 100%.

### Separation Medium or Separation Method (Aspect 23)

The goal of separation may, for example, be the analysis of incorporation events of modified nuc-macromolecules on the primer. When numerous different oligonucleotides (with primer-function) are present in the reaction, a simultaneous analysis of the results is desirable. A solid phase with immobilized oligonucleotides can fulfill the function of a separation medium, wherein the oligonucleotides can detect specific sequences in the oligonucleotide (that has appeared as primer). Such a solid phase can be present, for instance, as a single-dimensional or two-dimensional array (e.g., microarray). The purification of the solid phase can be conducted accordingly via washing of the arrays.

As another separation medium, gels can be used (e.g., agarose or polyacrylamide gels). Also, ultrafiltration, different kinds of chromatography (e.g., affinity chromatography) or spectroscopy (e.g., mass spectroscopy) can be used as separation methods.

### State of the Art for Control of the Polymerase Reaction.

1. Blockade of the 3′-Position

One possibility for controlling an enzymatic reaction consists in the use of modified substrates, for instance, dideoxy-nucleotides. The use of labeled dideoxy-nucleotides leads to an incorporation of only one nucleotide, because 3′-OH group needed for further synthesis is absent. The biggest disadvantage of this method of reaction control consists in an irreversible blockade of the synthesis on the given strand of the nucleic acid. The obvious consideration, to couple an easily cleavable group to the 3′-OH group and thereby reverse the termination, did not lead many researchers to the desired success. Many nucleotides modified in this way lost their substrate properties for the polymerases. Other modified nucleotides did not withstand the conditions of the enzymatic reaction and lost their markers during the synthesis (Canard et al. PNAS 1995 v.92 S.10859). The tight spatial relations in the active center of polymerases make it difficult to construct the desired modified nucleotides.

2. Steric Obstacle

Many low-molecular-weight markers used in the modern research represent a steric obstacle for the enzymes. Biotin, digoxigenin and fluorescence dyes like Fluoreszein, Tetramethylrhodamine, Cy3 dye are examples of a sterically demanding group (Zhu et al. Cytometry in 1997, v. 28, S.206, Zhu et al. NAR 1994, v. 22, S.3418, Gebeyehu et al., NAR 1987, v. 15, p. 4513, Wiemann et al. Analytical Biochemistry 1996, v. 234, p. 166, Heer et al. BioTechniques 1994 v. 16 p. 54). The distance between the marker (sterically demanding group) and the enzymatic active part of the molecule (nucleotide unit) amounts only to few Angstroms, because linkers consisting of 5 to 20 chain atoms are usually used. Depending on position and accessibility of the active enzymatic center of the enzyme (the active center may be deeply located inside in the interior of the enzyme or be on its surface) the low-molecular markers have a direct contact with the active center or stand in immediate proximity to it. The direct contact or also the nearness can lead to interference with the enzymatic process and, in case of polymerases, to an impairment of further synthesis. The direct contact or also the nearness of the low-molecular markers can also explain the influence of marker molecules on the enzymatic process (Tcherkassov WO 02088382).

In summary, it can be stated that only the possibilities of controlling the reaction within the polymerase, either by modification of a nucleotide components on the sugar (e.g., in 3′-OH position) or on the bases by low-molecular-weight ligands have so far been explored: terminating groups were either placed directly in the active center of the polymerase or in its immediate proximity. Besides, these chemical groups had a low molecular weight.

Individual molecular structures have their dimensions (e.g., length, width, height, volume etc.) in the range of several nanometers or even fractions thereof. Hence, even differences of few Angstroms or nanometers can cause a significant effect. For a differentiated consideration of potential mechanisms to control biologically active molecules these dimensions have to be taken into account. (In given case, polymerases can be considered as molecular copying machines).

In the context of the present invention, it was possible to influence and control the process of the enzymatic incorporation reaction by means of macromolecular ligands, wherein the macromolecules are not in the immediate neighborhood of the active center of the polymerase. In particular, the use of the present invention appears especially important in the new field of bionanotechnology and working with single molecules.

In one embodiment of invention, a method is provided to control the enzymatic synthesis reaction. This method is characterized by the application of modified nuc-macromolecules, which carry macromolecular sterically demanding ligands, in the enzymatic synthesis reaction. According to the invention, the macromolecular sterically demanding ligands have a molecular weight which amounts more than to 2 kDa. In this embodiment of the invention, the control of the enzymatic synthesis occurs through a sterically demanding macromolecular ligand, which is located outside of the polymerase molecule after the nucleotide component has been incorporated.

These relationships are depicted in FIGS. 8 to 11. The Figures are intended to merely schematically illustrate the inventive idea and do not claim completeness of the information. The incorporated modified nuc-macromolecule comprises a macromolecular sterically demanding ligand. This sterically demanding ligand does not permit another ligand to get close to the polymerase. With an appropriately selected linker length, no further modified nuc-macromolecule can be incorporated. The linker is depicted schematically in an extended state in its full length. In other words, the space-demanding properties of the sterically demanding ligand (for instance, caused by its size) prevent other modified nuc-macromolecules with similarly large ligands from getting near the active center of the polymerase. Further reaction is blocked.

Change in the spatial circumstances around the polymerase, for instance, by binding other proteins to the DNA or polymerase, can possibly lead to necessary changes in the linker length between the nuc-component and the steric obstacle.

In many cases, the following rule for the spatial potential for the linker length can be applied: the longer the linker length between the nuc-component and the sterically demanding ligand, the larger a sterically demanding ligand must be to prevent further synthesis. Smaller ligands can lose their effect as the linker length between the nucleotide component and steric ligand increases.

The principle of the method for controlling enzymatic incorporation by means of modified nuc-macromolecules will be explained using a model for the primer extension reaction as an example. This model is displayed schematically and serves only for clearness.
Following components are involved in the primer extension reaction:
   template-primer complex (Primer _{(N)})
   DNA-Polymerase
   nucleotides (unmodified or modified with a sterically demanding ligand).
   solution with buffer substances and divalent metal ions
The control of the progress of the enzymatic reaction is accomplished through the use of modified nuc-macromolecules.
After the incorporation of such a nucleotide analog, the sterically demanding ligand prevents the approach of another sterically demanding ligand to the nucleotide-binding center of the polymerase. But since another nuc-component is coupled to such a ligand via a relatively short linker, the approach of the nuc-component is also hindered, respectively. Therefore, the incorporation of another modified nuc-macromolecule becomes impossible.
Accordingly, the combination of the linker length and the steric properties of the ligand is important, wherein the following optimization strategies can serve for the choice of the appropriate combination. Both the sterically demanding ligand and the linker can be adjusted.
Strategy I: Default value is the sterically demanding ligand.
   For a given demanding ligand, different linker lengths should be tested:
      With a suitable linker, only one nucleotide analog is incorporated
      With too short a linker, the incorporation is completely inhibited
      With too long a linker, several nucleotide analogs with sterically demanding groups can be incorporated.
Strategy II: Default value is the linker.
   For a given linker, different sizes of the sterically demanding ligand should be tested:
      With a suitable ligand, only one nucleotide analog is incorporated
      With too small a ligand, several nucleotide analogs with sterically demanding groups can be incorporated.

As part of a reaction, all modified nuc-macromolecules can carry the same or also different sterically demanding ligands. The essential issue for reaction control is the effectiveness of the blocking effect of the ligands among one another.

In one embodiment, the control of the reaction includes the possibility of reversing the blockade of the reaction. Using known cleavable groups between the linker and the macromolecular sterically demanding ligand, the blockade can be reversed and further reaction can proceed.

### Applications

The method according to the invention for the step-by-step enzymatic synthesis reaction of nucleic acids can be used, for instance, in technologies for analysis of the genetic information (WO 02088382, DE 10 2004 025 696, DE 101 20 798, DE 102 14 395). In a preferred embodiment, this analysis is conducted at the single-molecule level, i.e. sequences of single molecules of nucleic acids are identified.

In a special embodiment, the method according to the invention is used in a method for the parallel sequence analysis of nucleic acid sequences, or nucleic acid chains (NAC), comprising the following steps:
1. Providing of a solid phase
2. Binding nucleic acid chains to be analyzed to the solid phase under formation of primer-template complexes capable of extension,
3. Carrying out a cyclic reaction with the primer-template complexes fixed on the solid phase comprising following steps:
   3.1 enzymatic incorporation of modified nuc-macromolecules to the formed primer-template complexes by means of a polymerase,
   3.2 washing of the solid phase
   3.3 Detection of the labeling of incorporated, labeled, modified nuc-macromolecules, wherein relative coordinates of individual signals are identified
   3.4 Removal of the signals from the incorporated, modified nuc-macromolecules,
   3.5 washing of the solid phase
   3.6 Repetition of the steps 3.1 to 3.5 if necessary
4. Reconstruction of the sequences of single nucleic acid chains from the signals obtained under step 3.3

The cyclic steps can be repeated several times, for instance, 2 to 10 times, 10 to 20 times, 20 to 100 times, or 100 to 500 times. The identification of the incorporated modified nuc-macromolecules is accomplished via markers.

The reaction conditions of the step (3) in a cycle are chosen so that the polymerases can incorporate a modified nuc-macromolecule on more than 50% NACF's participating in a sequencing reaction in one cycle (NACF-primer complexes capable of extension) or preferably on more than 80% or on more than 90% of complexes capable of extension. Accordingly, it is possible to vary the time, buffer and temperature conditions as well as the concentrations of reagents.

In one embodiment of the method, the polymerase and modified nuc-macromolecules are in the same solution or composition, which is added to the extendable complexes attached to the solid phase.

In another embodiment of the method, polymerases and modified nuc-macromolecules are provided in separated solutions or compositions. The solutions or compositions are separately added to the extendable complexes bonded to the solid phase. Accordingly, in a preferred embodiment, a solution or composition containing polymerase is added first, and a solution or composition containing a modified nuc-macromolecule is added thereafter (see example 15).

In some applications, a composition with one or several kinds of polymerase can be added in one step, and compositions with modified nuc-macromolecules are added in additional steps.

A method for the parallel sequence analysis of nucleic acid sequences (nucleic acid chains, NACs) is provided in a further embodiment of the invention, in which
fragments (NACFs) of single-stranded NACs with a length of approximately 50 to 1000 nucleotides that may represent overlapping partial sequences of a whole sequence are produced,
the NACFs are bonded to a reaction surface in a random order using a uniform primer or several different primers in the form of NACF-primer complexes, wherein the density of NACF primer complexes bonded to the surface allows for an optical detection of signals from single incorporated nuc-macromolecules,

a cyclical incorporation reaction of the complementary strand of the NACFs is performed using one or more polymerases by
a) adding, to the NACF primer complexes bonded to the surface, a solution containing one or more polymerases and one to four modified nuc-macromolecules that are labeled with markers (fluorescent marker), wherein the markers, which are each located on the nucleotide analogs when at least two nucleotide analogs are used simultaneously, are chosen in such a manner that the nucleotide analogs used can be distinguished from one another by measuring different fluorescent signals, wherein the nucleotide analogs comprise a macromolecular sterically demanding ligand, that the polymerase is not capable of incorporating another modified nuc-macromolecule in the same strand after such a modified nuc-macromolecule has been incorporated in a growing complementary strand, the marker being removable and the structural modification being a removable macromolecular sterically demanding ligand,
b) incubating the stationary phase obtained in step a) under conditions suitable for extending the complementary strands, the complementary strands each being extended by one modified nuc-macromolecule,
c) washing the stationary phase obtained in step b) under conditions suitable for removing nucleotide analogs that are not incorporated in a complementary strand,
d) detecting the single nucleotide analogs incorporated in complementary strands by measuring the characteristic signal of the respective marker (fluorescent marker), the relative position of the individual fluorescent signals on the reaction surface being determined at the same time,
e) cleaving off the marker and macromolecular sterically demanding ligands from the nucleotide analogs added to the complementary strand in order to produce unlabelled (NTs or) NACFs,
f) washing the stationary phase obtained in step e) under conditions suitable for the removal of the marker and ligand,
repeating steps a) to f), several times if necessary,

the relative position of individual NACF-primer complexes on the reaction surface and the sequence of these NACFs being determined by specific assignment of the fluorescent signals, which were detected in the respective positions in step d) during successive cycles, to the NTs.

The cyclic steps can be repeated several times, for instance, 2 to 10 times, 10 to 20 times, 20 to 100 times, 100 to 500 times, or 500 to 2000 times. The identification of the incorporated modified nuc-macromolecules is accomplished by means of the marker.

A suitable surface for such method can obtained according to DE 101 49 786 or DE 10 2004 025 744. The material preparation and the detection can be carried out according to WO 02088382, DE 10 2004 025 696, DE 101 20 798, or DE 102 14 395, DE 102 46 005.

### EXAMPLES

The presented examples and embodiments have the purpose of explaining the invention. The present invention is not limited to the embodiments and examples described here. Many different modifications of the invention in addition to those described here will appear obvious to a person skilled in the art. Such modifications should be credited to this invention, too.

The displayed individual embodiments should be considered in their entirety and can be combined with each other.

### General Suggestions for the Synthesis of Modified Nuc-Macromolecules

The modified nuc-macromolecules according to the invention can be synthesized in different ways. The order of the chemical steps during the coupling steps can vary. For instance, the linker component can be coupled to the nuc-component first, and the marker component together with the macromolecular sterically demanding ligand can be coupled afterwards. On the other hand, one or more linkers can be coupled to the macromolecular sterically demanding ligand and then to the nuc-component(s), after that the marker is coupled.

The coupling between individual components of modified nuc-macromolecules can be covalent or affine by its nature. The linking of individual components of the nuc-macromolecules can thereby be accomplished both by chemical and by enzymatical coupling. Couplings to amino or thiol groups represent examples of covalent binding (D. Jameson et al. Methods in Enzymology 1997, v. 278, p. 363-, “The chemistry of the amino group” S. Patai, 1968, “The chemistry of the thiol group” S. Patai, 1974). Biotin-streptavidin bonding, hybridization between complementary strands of nucleic acids or antigen-antibody interactions represent examples of affinity binding.

The macromolecular sterically demanding ligand and macromolecular markers often offer a variety of possibilities for coupling. One macromolecular ligand can have a number of coupling positions for the linkers, e.g. several binding sites for biotin, as is true in the case for streptavidin. A macromolecular marker or a macromolecular sterically demanding ligand can comprise several amino or thiol groups. The core component of a marker can be modified by a different number of signal-giving or signal-transmitting units. The exact ratio between these marker units can vary. Examples for the modification of polymers with dyes are known (Huff et al. U.S. Pat. No. 5,661,040, D. Brigati U.S. Pat. No. 4,687,732). If nucleic acids are used as macromolecular markers, they can comprise different parts for the coupling of other macromolecules. Other macromolecules, e.g. enzymes, can be bound to one macromolecular marker.

A modified nuc-macromolecule can carry macromolecular markers with different detection properties, for instance, a modified nuc-macromolecule can carry several dye molecules as well as sites for the affinity binding (e.g., via hybridization) of further macromolecules.

The coupling between the nuc-components and the linker components is preferably covalent. Many examples of a covalent coupling to nucleotides or their analogues are known (Jameson et al. Method in Enzymology, 1997, v. 278, p. 363-, Held et al. Nucleic acid research, 2002, v. 30 p. 3857-, Short U.S. Pat. No. 6,579,704, Odedra WO 0192284). The coupling can be accomplished, for instance, to phosphate, amino-, hydroxy- or mercapto groups.

Often, the linker component can be built up in several steps. In the first step, for instance, a short linker with a reactive group is coupled to the nucleotide or nucleoside, e.g., propargylamine-linker to pyrimidines Hobbs et al. U.S. Pat. No. 5,047,519 or other linkers, e.g. Klevan U.S. Pat. No. 4,828,979, Seela U.S. Pat. No. 6,211,158, U.S. Pat. No. 4,804,748, EP 0286028, Hanna M. Method in Enzymology 1996 v. 274, p. 403, Zhu et al. NAR 1994 v. 22 p. 3418, Jameson et al. Method in Enzymology, 1997, v. 278, p. 363-, Held et al. Nucleic acid research, 2002, v. 30 p. 3857-, Held et al. Nucleosides, nucleotides & nucleic acids, 2003, v. 22, p. 391, Short U.S. Pat. No. 6,579,704, Ward et al. U.S. Pat. No. 4,711,955, Engelhardt et al. U.S. Pat. No. 5,241,060 Taing et al. U.S. Pat. No. 6,811,979, Odedra WO 0192284, Herrlein et al. Helvetica Chimica Acta, 1994, V. 77, p. 586, Canard U.S. Pat. No. 5,798,210, Kwiatkowski U.S. Pat. No. 6,255,475, Kwiatkowski WO 01/25247, Parce WO 0050642, Faulstich et al. DE 4418691, Phosphoroamidite (Glen Research Laboratories, http://www.glenres.com/, Trilink Biotechnologies, S. Agrawal “Protocols for oligonucleotide conjugation”, Humana Press 1994, M. Gait “Oligonucleotide synthesis: a practical approach” IRL Press, 1990), dissertation “Synthese basenmodifizierter Nukleosidtriphosphate und ihre enzymatische Polymerisation zu funktionalierter DNA”, Oliver Thum, Bonn 2002.

Some compounds are commercially available, e.g., from Trilink Biotechnologies, Eurogentec, Jena Bioscience.

These short linkers serve as coupling units L or their parts, and are constituents of the linker component in the completed modified nuc-macromolecule.

The coupling of the nucleotide or nucleoside with a short linker to a linker-polymer can be accomplished in the second step. Polymers with reactive functional groups are commercially available (Fluka).

After the coupling of the nucleotide to the polymer, the marker component now can be coupled as the last step.

It is often advantageous to couple a short linker to a nucleoside and then, if necessary, to convert this modified nucleoside into a nucleoside triphosphate (synthesis of triphosphates can be found, for instance, in the following citations: Held et al. Nucleosides, nucleotides & nnucleic acids, 2003, v. 22, p. 391, Faulstich et al. DE 4418691, T. Kovacs, L. Ötvös, Tetrahedron Letters, Vol 29, 4525-4588 (1988) or dissertation “Synthese basenmodifizierter Nukleosidtriphosphate und ihre enzymatische Polymerisation zu funktionalierter DNA”, Oliver Thum, Bonn 2002). Further modifications can be carried out with nucleoside triphosphate analogs.

Precursors for modified nucleosides are available, for instance, from Trilink Biotechnologies (San Diego, APPROX., the USA) or from Chembiotech (Muenster, Germany).

Coupling of macromolecular sterically demanding ligands can occur in different way. For instance, macromolecular sterically demanding ligands can first be coupled to the structure consisting of nuc-linker and the coupling to the marker takes place only subsequently. Another approach starts with the primary coupling of sterically demanding ligands to the marker (e.g., coupling of streptavidin to phycoerhytrin) followed by the coupling to the structure consisting of nuc-linker. The macromolecular sterically demanding ligand can also appear as a component of the marker, e.g., as a core component. In this case, low-molecular-weight substances (e.g., dyes, e.g., Cy3) can be directly or indirectly (e.g., by another linker) coupled to the ligand, see examples.

The coupling between the linker component and the marker component can occur, for instance, between the marker component and the reactive groups on the linker component. Reagents for such couplings are described in detail in “Chemistry of protein conjugation and crosslinking”, S. Wang, 1993, ISBN 0-8493-5886-8. The abovementioned patents also describe the methods for handling and coupling several macromolecules for different types of macromolecules. Further examples (for proteins) of couplings to and between the macromolecules are described in “Bioconjugation: protein coupling techniques for the biomedical sciences”, M. Aslam, 1996, ISBN 0-333-58375-2; “Reactive dyes in protein an enzyme technology”, D. Clonis, 1987, ISBN 0-333-34500-2; “Biophysical labeling methods in molecular biology” G. Likhtenshtein, 1993, 1993, ISBN 0-521-43132-8; “Techniques in protein modification” R. Lundblad, 1995, ISBN 0-8493-2606-0; “Chemical reagents for protein modification” R. Lundblad, 1991, ISBN 0-8493-5097-2; for nucleic acids in “Molecular-Cloning”, J. Sambrook, Vol. 1-3, 2001, ISBN 0-87969-576-5, for other types of polymers in “Makromoleküle, Chemische Struktur and Synthesen”, Vols. 1, 4, H. Elias, 1999, ISBN 3-527-29872-X.

Because the marker component usually comprises many coupling positions, it is possible to carry out further modifications with the assembled modified nuc-macromolecules. For instance, further modifications can block or change excess free amino groups.

Depending on the field of application and reaction conditions under which modified nuc-macromolecules are used, different types of chemical bonds between separate parts of the macromolecules can be advantageous.

In the following, some possible methods for synthesis of modified nuc-macromolecules will be described for the sake of example. These are not intended to restrict the possible synthesis paths or to restrict the possible modified nuc-macromolecule structures.

The following provides examples of modified nuc-macromolecules with polyethylene glycol (PEG) as a linker component. Examples of the coupling of PEG to other molecules are shown in “Poly(ethylene glycol): chemistry and biological applications”, 1997. In particular, very different reactive groups can be used for the coupling: N-succinimidyl carbonate (U.S. Pat. No. 5,281,698, U.S. Pat. No. 5,468,478), amines (Buckmann et al. Makromol. Chem. V.182, p. 1379 (1981), Zalipsky et al. Eur. Polym. J. V.19, p. 1177 (1983)), succinimidyl propionate and succinimidyl butanoate (Olson et al. in Poly(ethylene glycol) Chemistry & Biological Applications, 170-181, Harris & Zalipsky Eds., ACS, Washington, D.C., 1997; U.S. Pat. No. 5,672,662), succinimidyl succinate (Abuchowski et al. Cancer Biochem. Biophys. v. 7, p. 175 (1984), Joppich et al., Makromol. Chem. 1v. 80, p. 1381 (1979), benzotriazole carbonate (U.S. Pat. No. 5,650,234), glycidylether (Pitha et al. Eur. J. Biochem. v. 94, p. 11 (1979), Elling et al., Biotech. Appl. Biochem. v.13, p. 354 (1991), oxycarbonylimidazole (Beauchamp, et al., Anal. Biochem. v.131, p. 25 (1983), Tondelli et al. J. Controlled Release v.1, p. 251 (1985)), p-nitrophenyl carbonate (Veronese, et al., Appl. Biochem. Biotech., v.11, p. 141 (1985); and Sartore et al., Appl. Biochem. Biotech., v.27, p. 45 (1991)), aldehyde (Harris et al. J. Polym. Sci. Chem. Ed. v.22, p. 341 (1984), U.S. Pat. No. 5,824,784, U.S. Pat. No. 5,252,714), maleimide (Goodson et al. Bio/Technology v.8, p. 343 (1990), Romani et al. in Chemistry of Peptides and Proteins v.2, p. 29 (1984)), and Kogan, Synthetic Comm. v.22, p. 2417 (1992)), orthopyridyl-disulfide (Woghiren, et al. Bioconj. Chem. v. 4, p. 314 (1993)), Acrylol (Sawhney et al., Macromolecules, v. 26, p. 581 (1993)), Vinylsulfone (U.S. Pat. No. 5,900,461). Additional examples for coupling PEG to other molecules are shown in Roberts et al. Adv. Drug Deliv. Reviews v. 54, p. 459 (2002), U.S. Patent No. 2003124086, U.S. Patent No. 2003143185, WO 03037385, U.S. Pat. No. 6,541,543, U.S. Patent No. 2003158333, WO 0126692.

Other similar polymers can be coupled in a similar way. Examples of such polymers are poly(alkylene glycol), copolymers of ethylene glycol and propylene glycol, poly(olefinic alcohols), poly(vinylpyrrolidone), poly(hydroxyalkylmethacrylamide), poly(hydroxyalkyl methacrylate), poly(saccharide), poly(x-hydroxy acids), poly(acrylic acid), poly(vinyl alcohol).

The purification of the modified nuc-components of the nuc-macromolecules is accomplished using conventional means of nucleotide chemistry: for instance, with silica gel chromatography in a water-ethanol mixture, ion exchange chromatography in a salt gradient and reverse-phase chromatography in a water-methanol gradient. Sigma-Aldrich, for example, offers optimized chromatography columns for nucleotide purification.

The purification of macromolecular linker components and marker components can be performed through ultrafiltration, gel electrophoresis, gel filtration and dialysis, see “Bioconjugation: protein coupling techniques for the biomedical sciences”, M. Aslam, 1996, ISBN 0-333-58375-2.

The mass of the modified nuc-macromolecules differs substantially from the mass of the nucleotides. For this reason it is advantageous to use the ultrafiltration for the final purification steps. Since only an average mass is calculated for the modified nuc-macromolecules, ultrafiltration is also suitable as an analytic method for separation of synthesis products.

It is possible to apply different methods of the macromolecular chemistry for the characterization of the modified nuc-macromolecules, e.g., UV-vis spectroscopy, fluorescence measurement, mass spectroscopy, fractionation, size exclusion chromatography, ultracentrifugation and electrophoretic technologies, like IEF, denaturating and non-denaturating gel electrophoresis (“Makromoleküle, Chemische Struktur and Synthesen”, Band 1, 4, H. Elias, 1999, ISBN 3-527-29872-X, “Bioconjugation: protein coupling techniques for the biomedical sciences”, M. Aslam, 1996, ISBN 0-333-58375-2).

The propertiesof biotin-streptavidin bond are described in Gonzalez et al. Journal Biolog. Chem. 1997, v. 272, p. 11288

### Synthesis of Modified Nucleotides

### Methods for Separation

### Thin Layer Chromatography, TLC:

Analytical TLC: “DC-Alufolien 20×20 cm Kieselgel 60 F 254” (VWR, Germany), coated with fluorescent indicator. Visualization was conducted with UV light. Separation medium: ethanol/water mixture (70:30), (separation medium, German “Laufmittel”, LM 1) or ethanol/water (90:10), LM2. Preparative TLC plates: silica gel plates with collecting layer (VWR, Germany). LM 1 or LM 2.

### Reverse-Phase Chromatography (RP Chromatography), RP-18:

C-18 material (Fluka, Germany), column volume 10 ml, water/methanol gradient. Fractions, each 10 ml, were collected and analyzed with a UV-vis spectrometer. Fractions with similar spectra were combined and lyophilized. HPLC columns with the same material can also be used.

### Ion-Exchange Chromatography:

DEAE cellulose (VWR, Germany), gradient NH₄HCO₃ 20 mmol/l to 1 mol/l, fractions were collected under UV/vis-control; those with similar spectra were combined.

Affinity isolation can be used for purification of modified nuc-macromolecules, e.g. if there are oligonucleotides as a part of the marker component. Such selective isolation can be accomplished for example via a hybridization on the complementary nucleic acid immobilized on a solid phase.

Estimation of the yields of the dye-marked product was conducted with UV-vis spectrometry.

An estimation of saturation degree of the binding to streptavidin was conducted via a control titration with biotin dye (biotin-4-fluorescein, Sigma), 100 μmol/l in 50 mmol/l borate buffer, pH 8, for 5 min at RT. If all potential sites for binding were saturated during the synthesis, there would be no binding of biotin dye to the streptavidin. In the case of insufficient reaction, there would be binding of biotin dye that can be measured by UV-vis.

### Material

dUTP-AA (dUTP-allyl-amine, Jena Bioscience), dCTP-PA (dCTP-propargyl-amine, Jena Bioscience), dATP-PA (7-(3-Amino-1-propynyl)-2′ deoxy-7-deazaadenosin-5′-Triphosphat) (custom synthesis by Jena Bioscience), dGTP-PA (7-(3-Amino-1-propynyl)-2′ deoxy-7-deazaguanosin-5′-Triphosphat, (custom synthesis by JenaBioscience), PDTP (3-(2-pyridinyl-dithio)-propionic acid, Fluka), 7-(3-phthalimido-1-propynyl)-2″-deoxy-7-deazaguanosine and 7-(3-phthalimido-1-propynyl)-2″-deoxy-7-deazaadenosine (Chembiotech), PDTP-NHS (3-(2-pyridinyl-dithio)-propionic acid-N-hydroxysuccinimidyl ester, Sigma), Cy3-NHS (Cy3-N-hydroxysuccinimidyl ester, Amersham Bioscience), MEA (mercaptoethylamine, Sigma), DTT (1,4-dithio-DL-threitol, Sigma), CA (cystamine, Sigma), TCEP (tris-(2-carboxyethyl)phosphine, Sigma), biotin-NHS (biotin-N-hydroxysuccinimidyl ester, Sigma). 3-Ac (iodoacetate, Sigma), iodacetamide (Sigma), EDA (ethylendiamine, Sigma), CDI (1,1′-carbonyldiimidazole, Sigma), EDC N-(3-Dimethylaminopropyl)-N-Ethylenecarbodiimide (Sigma), NH2-PEG-Biotin (30 atoms), Sigma), biotin-PEG-NHS (5,000 Da, Nektar), SA (streptavidin, Roche), SA-PE (Streptavidin-Phycoerythrin, Molecular Probes Inc.), Biotin-PEG(8)-SS-PEG(8)-Biotin (Kat. No. PEG1064, IRIS Biotech GmbH), Fluorescein-PEG-NHS (5000 Da, Nektar), BOC-PEG-NHS (3000 Da, Nektar), Fmoc-PEG-NHS (5000 Da, Nektar), dUTP-16-Biotin (Roche), nonmodified, natural nucleotides (Roth)

### List of Suppliers and Companies:

Aldrich—see Sigma
Amersham—Amersham Bioscience, Freiburg, Germany
Chembiotech—Chembiotech, Münster, Germany
Fluka—see Sigma
Jena Bioscience—Jena Bioscience, Jena, Germany
Molecular Probes—Molecular Probes Europe, Leiden, Netherlands
MWG—MWG Biotech, Ebersberg near Munich, Germany,
Nektar—Nektar Molecular Engineering, previous Shearwater Corporation, Huntsville, Ala., USA
Quantum Dot—Quantum Dot Hayward, Calif., USA
Roche—Roche, Mannheim, Germany
Sigma—Sigma-Aldrich-Fluka, Taufkirchen, Germany
Trilink—Trilink Biotechnologies Inc. San Diego, Calif., USA,

Organic solvents were purchased from Fluka at p.a. purity grade or were dried according to standard procedures. For solvent mixtures, the mixing ratio is stated in terms of volume to volume (v/v).

### Example 1

### dUTP-AA-PDTP, FIG. 12

### (The Synthesis was Conducted Similar That Described in WO 2005 044836)

dUTP-AA (20 mg) was dissolved in 1 ml of water and the pH value was adjusted to 8.5 with NaOH. PDTP-NHS (60 mg dissolved in 0.5 ml methanol) was added dropwise to this aqueous solution of dUTP-AA under stirring. The reaction was carried out at 40° C. for 2 hours. TLC Analysis: dUTP-AA-PDTP (in LM 1 Rf 0.45).

The isolation of the product from excess of PDTP-NHS and PDTP was performed on preparative TLC plates, LM 2. The resulting products, dUTP-AA-PDTP and dUTP-AA, were eluted from the plate with water and dried.

This dUTP analog comprises a disulfide bond that can react with other thiols in a thiol exchange reaction under mild conditions resulting in a formation of a new cleavable bond.

This example illustrates a general possibility of introducing further modifications into the nucleotides. Other base-modified nucleotide analogs, such as 7-deaza-aminopropargyl-deoxy-guanosine triphosphate, 7-deaza-aminopropargyl deoxy-adenosine triphosphate, 5-aminopropargyl-deoxy-uridine triphosphate, 5-aminoallyl-deoxy-uridine triphosphate, and 5-amino-propargyl-deoxy-cytidine triphosphate, can be modified in the same way.

### Example 2

### dUTP-AA-Propionate-SH, FIG. 13

One ml of aqueous TCEP solution, 250 mmol/l, pH 8, adjusted with NaOH, was added to 200 μl 40 mmol/l aqueous solution of dUTP-AA-PDTP, and the reaction was allowed to proceed for 10 min at RT under stirring. The separation of nucleotides from other reagents took place on preparative TLC plates, LM 2. Under these conditions the product, dUTP-AA-propionate-SH, remains on the starting line. The modified nucleotides were eluted from the plate with water and dried.

This dUTP analog comprises a reactive SH group that can be easily modified, e.g. by thiol exchange reaction resulting in a new disulfide bond.

### Example 3

### Biotin-PEG-Ethyl-SH, FIG. 14

Biotin-PEG-NHS (10 mg, 5000 Da Nektar) was added to 200 μl aqueous CA solution (100 mmol/l), pH 8.5, adjusted with NaOH; the reaction proceeded at 40° C. for 18 hours under stirring. Then 200 μl of TCEP solution (0.5 mol/l), pH 8.0, was added and the reaction was allowed to proceed for a further 10 min at RT under stirring. The product was separated from low-molecular-weight compounds by ultrafiltration at a MWCO (Molecular weight cutoff) of 3,000.

The product comprises a reactive SH group that can be easily modified, e.g. by thiol-exchange reaction resulting in a new disulfide bond.

A further example of introduction of a SS-Bond or a mercapto group to PEG:

Ten mg of amino-PEG-biotin (PEG-linker with 30 atoms, Sigma Aldrich) were dissolved in 280 μl of 50-mM borate buffer and pH was adjusted to 9. Two equivalents of PDTP-NHS, dissolved in 100 μl of DMF, were added to the resulting solution. After 1 hour at RT, the excess PDTP-NHS was reacted with the excess of NH₄HCO₃. Biotin-PEG-PDTP is the resulting product. This product can be coupled to another molecule by thiol exchange. A free SH group can be generated by cleavage of the SS bond.

Further linkers with reactive groups, like carboxy, thiol, disulfid groups with a PEG-spacer (e.g. Biotin-PEG(8)-SS-PEG(8)-Biotin) can be purchased from IRIS-Biotech GmbH (Germany).

### Example 4

### dUTP-AA-PEG-Biotin, FIG. 15

(The Synthesis was Conducted Similar that Described in WO 2005 044836)

Biotin-PEG-NHS (10 mg, 5000 Da, Nektar) was added to 100 μl aqueous solution of dUTP-AA, 50 mmol/l, pH 8.0, and stirred at 40° C. for 18 h. Next, the unreacted nucleotide was separated by ultrafiltration, 3,000 MWCO, and the product, dUTP-AA-PEG-biotin, was thoroughly washed with water.

This compound comprises a nucleotide functionality and a macromolecular linker. Biotin represents the coupling unit (T). Macromolecular structures can be coupled to this coupling unit (T), e.g. streptavidin, or proteins or beads modified with streptavidin.

This product is an intermediate compound for a modified nuc-macromolecule. This example shows that it is generally possible to modify nucleotides. Other base-modified nucleotide analogs, e.g. 5-propargylamino-dCTP, 7-deaza-aminopropargyl-dGTP, 5-amino-propargyl-dUTP and 7-deaza-aminopropargyl-dATP can be modified in a manner similar to the described procedure. Ribonucleotides, 2′-deoxyribonucleotide or 2′,3′-dideoxyribonucletide can be used (FIGS. 16, 21 to 24).

### Example 5

### dUTP-AA-SS-PEG-Biotin, FIG. 17,

A solution of dUTP-AA-PDTP (50 μl, 30 mmol/l in 50 mmol/l borate, pH 9.5) was added to a solution of Biotin-PEG-Ethyl-SH (100 μl, 10 mmol/l in 50 mM borate, pH 9.5). The reaction mixture was stirred for 18 hours at RT. The separation steps were conducted as described for the synthesis of dUTP-AA-PEG-Biotin (example 4).

This compound comprises a nucleotide functionality and a macromolecular linker. Biotin acts as a coupling unit (T). Macromolecular structures can be coupled to this coupling unit (T), e.g. streptavidin. Further macromolecules can be coupled via streptavidin, e.g. enzymes or nucleic acids.

The linker component can be cleaved off simultaneously with the marker component under mild conditions. This can be advantageous for methods like sequencing by synthesis (Balasubramanian WO 03048387, Tcherkassov WO 02088382, Quake WO0132930, Kartalov WO02072892), where removal of the marker is necessary after each detection step.

### Example 6

### dCTP-PA-SS-(PEG)8-Biotin

Step 1: First, dCTP-Pa was modified with PDTP-NHS, resulting in dCTP-PA-PDTP. The synthesis was carried out similarly to that for dUTP, see example 1.

Step 2: An aqueous solution of TCEP (10 μl, 300 mmol/l, pH 7, adjusted by NaOH) was added to an aqueous solution of biotin-PEG(8)-SS-PEG(8)-biotin (50 μl, 100 mmol/l, pH6, Iris Biotech GmbH). This cleaves off approximately half of the disulfide bridges.

An aqueous solution of dCTP-PA-PDTP (20 μl, 20 mmol/l, pH 9.5, adjusted by NaOH) was added to the solution obtained in step 2 and incubated for 1 hour at RT. The product was isolated via thin layer chromatography (TLC) using LM 1. The nucleotides were eluted from the plate with water and evaporated.

### Example 7

### Synthesis of Macromolecular Sterically Demanding Ligands

This example is intended to demonstrate variations in size and the labeling of macromolecular sterically demanding ligands. The following modifications of streptavidin were synthesized:

### SA-(PEG (3,000-BOC))ₙ,

### SA-(PEG (5,000-Fmoc))ₙ,

SA-(PEG (5,000-fluorescein)) n

Streptavidin (Promega Inc) and BOC-PEG-NHS (3000 Da, Nektar), Fmoc-PEG-NHS (5000 Da, Nektar) and fluorescein-PEG-NHS (5000 Da, Nektar) served as starting material. PEG derivatives were added to a solution with streptavidin (5 mg/ml in 50 mmol/l borate buffer, pH 9) up to a concentration of 10% (w/v) and incubated for approx. 2 hr at RT. The modified streptavidin was purified from the excess of PEG derivatives by means of ultrafiltration. On average, every SA molecule was modified with 10 PEG molecules (n=10). The size of other protein conjugates can be also be similarly changed in a graduated manner, it being possible to use high-molecular PEG derivatives

Nucleotide Analogs with a Macromolecular Sterically Demanding Ligand.

A controlled enzymatic synthesis of nucleic acids (stepwise primer-extension) comprises a controlled stop, purification of nucleic acids and, if necessary, removal of the stop and continuation of the synthesis. The stop in the synthesis is caused by incorporating nucleotide analogs with macromolecular sterically demanding ligands according to the invention. Interrelationships between the linker length and the extent of steric obstacle will be demonstrated using several examples of nucleotide analogs.

### Example 8

### (dUTP-16 Biotin)4-SA,

A solution of streptavidin (200 μl, 1 mg/ml, in 50 mmol/l Tris-HCl, pH 8.0) was added to a solution of Biotin-16-dUTP, having linker length 16 atoms, (200 μl, 200 μmol/l, in 50 mmol/l Tris-HCl, pH 8.0). After 1 hour at RT, the (dUTP-16 Biotin)4-SA was separated from non-reacted Biotin-16-dUTP by ultrafiltration, 50,000 MWCO.

A compound was obtained which displays both a nucleotide functionality and a macromolecular sterically demanding ligand. This ligand cab be considered also as a marker.

This compound is not accepted by polymerases (e.g., Klenow—Exo-minus polymerase and terminal transferase) as a substrate. The modification leads to the loss of substrate properties.

Evaluation of the nucleotide structure shows that the linker is too short for this macromolecular structure (streptavidin). In this combination, the streptavidin, as a macromolecular ligand, does not allow the nucleotide component to get close enough to the active center of the Klenow fragment.

### Example 9

### Synthesis of dUTP-AA-PEG-Biotin-SA Derivatives

The following streptavidin derivatives (SA derivatives) were used in the synthesis: SA-(PEG (3,000-BOC))ₙ, SA-(PEG (5,000-Fmoc))ₙ, SA-(PEG (5,000-Fluorescein))ₙ

A solution with streptavidin derivatives (two equivalents) was added to a solution with dUTP-AA-PEG-biotin (100 μl, approx. 150 μmol/l) and agitated at RT for 1 h. After that, the product is purified by ultrafiltration, 50,000 MWCO, and is washed twice with water. Compounds comprising a nucleotide functionality, a long macromolecular linker and a macromolecular sterically demanding ligand were obtained.

A compound of dUTP-AA-PEG-biotin-SA-PE was obtained in a similar way.

It is advantageous to couple a single nuc-component to SA derivatives. This can be accomplished for example by an excess of SA derivatives. A population represents a mixture of SA derivatives, nucleotide-SA-derivatives and (nucleotide)n-SA-derivatives, wherein the form of nucleotide-SA prevails over the (nucleotide)n-SA-derivative for appropriate choice of molar ratios. The ratio between the nucleotide portion and that of modified streptavidin (dNTP:SA) can be in the following ranges: 0.01:1 to 0.1:1; 0.1:1 to 0.5:1; 0.5:1 to 1:1; 1:1 to 2:1, 2:1 to 3:1; and 3:1 to 4:1. Since such nucleotide compounds still have free biotin-bonding valences for biotin, further structures, e.g., biotin carrying signal-giving structures such as dyes or quantum dots, can be coupled over them.

The following compounds were obtained:

dUTP-AA-PEG-biotin-SA-(PEG(3,000-BOC))ₙ
dUTP-AA-PEG-biotin-SA-(PEG(5,000-Fmoc))ₙ
dUTP-AA-PEG-biotin-SA-(PEG(5,000-fluorescein))ₙ
dUTP-AA-PEG-biotin-SA-PE

Compounds comprising dCTP-derivatives were synthesized in a similar way.

The compound dUTP-AA-PEG-biotin-SA-(PEG(5,000-fluorescein))ₙ has a macromolecular ligand which is modified with dyes (fluorescein). Other dyes can be coupled either directly to the streptavidin or via linkers. SA-(PEG (5,000-Fmoc))n can be modified, for instance, on liberated amino groups with NHS derivatives of dyes after Fmoc-protective groups have been removed. In this manner, the macromolecular sterically demanding ligand can also have a marker function.

These compounds are accepted by polymerases as substrates, e.g. Klenow exo-minus polymerase, Sequenase, Vent exo-minus, Taq polymerase, Pwo polymerase, reverse transcriptase (MMLV (Promega), ImProm II™ (Promega)).

The effectiveness of the obstruction of the enzymatic synthesis was tested in the example with homopolymer regions in the template and with the polymerase Vent exo-minus: during the enzymatic synthesis on complementary template positions where a multiple incorporation of these nucleotide analogs could be possible (e.g., AAA segments, homopolymers), an incorporation of up to three successive modified nucleotide analogs dUTP-AA-PEG-biotin-SA-(PEG(3,000)-BOC) can be observed. However, the fraction of the primer in which a multiple extension took place is small (efficiency of termination on homopolymer regions is more than 90%).

For a given linker length, a complete stop can be achieved by enlarging the sterically demanding ligand. Vent exo-minus incorporates the analogs dUTP-AA-PEG-biotin-SA-(PEG (5,000-fluorescein))n and dUTP-AA-PEG-SA-PE on homopolymer segments only once. The nucleotide analog that has already been incorporated completely blocks the incorporation of the next complementary nucleotide analog (efficiency of termination on homopolymer regions is more than 99%).

Not only identical macromolecular sterically demanding ligands, but those of the similar size also lead to obstruction in the incorporation of modified nucleotide analogs.

When dUTP-AA-PEG-biotin-SA-(PEG (3,000)-BOC)ₙ and dCTP-AA-PEG-biotin-SA-(PEG (5,000-Fluorescein))ₙ are both available in a reaction solution at the same time, the dUTP-analog leads to obstruction in the incorporation of the dCTP analog on segments where first dU and then dC are to be incorporated. This shows that several modified nuc-macromolecules can be present in the reaction mixture at the same time and that only one complementary modified nuc-macromolecule is incorporated into the primer nevertheless.

### Example 10

### Bead-(SA-(dUTP-AA-PEG-Biotin))n

A 1%-suspension of streptavidin-coated polystyrene beads (1000 μl, diameter of 0.86 μm) was incubated with a solution of dUTP-AA-PEG-biotin (10 μl, 1 mmol/l) for 10 min at RT. The purification of the beads was accomplished by centrifugation for 5 min at 10000 rpm and a buffer exchange (10× with 200 μl of Tris-HCl 50 mmol/l, pH 8.5).

The resulting nucleotide-modified beads can be incorporated in/coupled to a nucleic acid chain by Klenow fragment.

The influence of such a steric obstacle differs from the effect of the sterically demanding ligands with a mass between 20,000 Da and 10,000,000 Da (e.g., proteins and their complexes). Since the space requirement of a nanobead/or a nanoparticle can amount to several hundred nanometers, such a sterically demanding ligand can make not only immediately adjacent areas of the nucleic acid, but also substantially larger areas of the nucleic acid inaccessible for the coupling of another modified molecule of similar size.

### Example 11

### Making a Nucleotide Analog with a Macromolecular Steric Obstacle

### (Linker 43 Atoms).

dCTP-PA-SS-(PEG)₈-biotin-SA-Cy3,
dCTP-PA-SS-(PEG)₈-biotin-SA-PE

The coupling of dCTP-PA-SS-(PEG)₈-biotin to streptavidin was carried out similarly as described for dUTP-AA-PEG-biotin. One equivalent of dCTP-PA-SS-(PEG)₈-biotin was added to 1.5 equivalents of streptavidin (aqueous solution, 5 mg/ml, in 50 mmol/l Tris-HCl, pH 8.0). After 1 hrs at RT, the resulting dCTP-PA-SS-(PEG)₈-biotin-SA was purified from substances of lower molecular weight by ultrafiltration with MWCO 50,000.

Then, the dCTP-PA-SS-(PEG)₈-biotin-SA was modified by Cy3-NHS in borate buffer (50 mmol/l, pH 8.5), so that on average 3 to 5 Cy3 molecules were coupled per streptavidin molecule (FIG. 18). There resulted a mixture of several modifications of dCTP-PA-SS-(PEG) 8-biotin-SA-Cy3. This mixture was not separated further.

This nucleotide analog was accepted as substrate by several polymerases, e.g., Klenow fragment exo-minus polymerase, Sequenase 2, Vent exo-minus, Taq polymerase, Pwo polymerase.

During the enzymatic synthesis on complementary template positions, where a multiple incorporation of these nucleotide analogs was possible (e.g., GGG segments, homopolymer regions in the template), the incorporation of only one nucleotide analog could be detected (see example 15). In this case, the sterically demanding macromolecular ligand (in this example streptavidin) has a restraining effect on the further enzymatic reaction: other nucleotide analogs (in this case dCTP-PA-SS-(PEG)₈-biotin-SA) could not be incorporated in the position adjacent to the primer_{(N+2)}. After the cleavage of the linker by reduction of the disulfide bond and the subsequent blockade of the SH group with iodacetamide, another dCTP-PA-SS-(PEG)₈-biotin-SA) could be incorporated. For an example of carrying out such a reaction, see example 15.

Other molecules can be coupled to the streptavidin. Instead of streptavidin, commercially available streptavidin conjugates can be used in the abovementioned synthesis, for instance, Streptavidin-PE (Molecular Probes Inc Invitrogen), Streptavidin-AP, Streptavidin-HRP or fluorescence dye conjugates (FIG. 19).

For instance, the synthesis of dCTP-PA-SS-(PEG)8-biotin-SA-PE was carried out similarly to that of dCTP-PA-SS-(PEG)8-biotin-SA: One equivalent of dCTP-PA-SS-(PEG)8-biotin was added to an equivalent of Streptavidin-PE, Molecular Probes, (aqueous solution, 1 mg/ml, in the manufacturer's buffer). After 1 hrs at RT, the resulting dCTP-PA-SS—(PEG)8-biotin-SA-PE was purified of substances with lower molecular weight by ultrafiltration with MWCO 100,000.

This nucleotide analog was accepted by many polymerases too (see above) and, after an incorporation (for example on homopolymer regions), leads to obstruction in the incorporation of another dCTP-PA-SS-(PEG)₈-biotin-SA-PE in the immediate vicinity

### Example 12

### A Further Example for the Synthesis of Modified Nuc-Macromolecule SA-(dGTP-PA-SS-PEG-Biotin)n

Manufacturing of a Nucleotide Analog with a Macromolecular Sterical Hindrance (Linker 43 Atoms). (n) Ranges from 1 to 4.

A solution of streptavidin (200 μl, 50 μmol/l, in 50 mmol/l borate buffer, pH 9) was incubated with 5 equivalents of biotin-PEG-PDTP (PEG linker 30 atoms; for synthesis, see example 3) for 10 minutes. Streptavidin-(biotin-PEG-PDTP)n was separated from low-molecular-weight components via ultrafiltration with a 30 kDa MWCO filter by repeated washings with borate buffer. A solution of TCEP (100 μl, 10 mmol/l, pH 8) was added to the solution of streptavidin-(biotin-PEG-PDTP)n (200 μl, 50 μmol/l) in borate buffer. After 30 min, streptavidin-(biotin-PEG-R-SH)n was again separated from low-molecular-weight components via ultrafiltration on 30 kDa MWCO by repeated washings with borate buffer.

dGTP-PA was modified with PDTP-NHS similar as described in example 1; the product is dGTP-PA-PDTP. A solution of dGTP-PA-PDTP (50 μl, 100 mmol/l, in 50 mmol/l borate buffer, pH 9) was added to the solution of streptavidin-(biotin-PEG-R-SH)4 (200 μl, 50 μmol/l, in 50 mmol/l borate buffer). After 30 min at RT, macromolecular products, including SA-(dGTP-PA-SS-PEG-Biotin)ₙ were separated from low-molecular-weight components via ultrafiltration on 30 kDa MWCO by repeated washings with borate buffer.

The resulting product SA-(dGTP-PA-SS-PEG-Biotin)ₙ has a linker of 43 atoms between the nuc-component and the biotin. This modified nuc-macromolecule has a cleavable SS-bond in its linker and can be incorporated into a nucleic acid chain by Klenow fragment.

During the enzymatic synthesis (Klenow exo-minus polymerase) on complementary template positions where a potentially multiple incorporation of these nucleotide analogs could occur (e.g., —CCC— segments in the template), the incorporation of only one nucleotide analog could be detected. In this case, the sterically demanding macromolecular ligand (streptavidin) has a restraining effect on the further enzymatic reaction: other nucleotide analogs (in this case SA-(dGTP-PA-SS-PEG-biotin)ₙ) could not be incorporated in the immediate vicinity. The linker component and the sterically demanding ligand can be cleaved-off from the nuc-component under mild conditions.

Other molecules can be coupled to the streptavidin. Instead of streptavidin, commercially available streptavidin conjugates can be used in the abovementioned synthesis, for instance, Streptavidin-AP, Streptavidin-HRP or fluorescence dye conjugates (FIG. 18 or FIG. 19). These conjugates, attached to a modified nuc-macromolecule, have a similar effect as streptavidin itself.

### Example 13

The combination of modified linkers carrying biotin with modified streptavidins as sterically demanding ligands represents an example for the synthesis of other modified nucleotides:

dUTP-AA-SS-PEG-biotin (synthesis, see example 5) and, in similar way, synthesized dCTP-PA-SS-PEG-biotin, dATP-PA-SS-PEG-biotin and dGTP-PA-SS-PEG-biotin can be combined with different variations of the steric obstacle and markers:

Modification with SA-(PEG-X)_{N}:

Where N=3-12 and (X) comprises, for instance, a dye, e.g., FITC, Cy3, Cy5 rhodamine (for examples of further dyes, see catalog of Dyomics GmbH, Jena, Germany or Molecular Probes, Invitrogen), or a protective group, e.g., Fmoc, or an amino group. The dyes can be coupled via PEG as well as directly to the streptavidin. It is also possible to change the size of further SA modifications (e.g., SA-PE) in a graduated manner using polymers (as for example PEG, different commercially available PEG derivatives, e.g., Sigma-Aldrich-Fluka, Iris Biotech, their sizes can range e.g., between 500 and 10000 Da) and to extend them using other modifications, e.g., by coupling dyes.

The following compounds represent an example of reversible terminators with macromolecular sterically demanding ligands:

dUTP-AA-SS-PEG-biotin-SA-(PEG-X)_{N},
dCTP-PA-SS-PEG-biotin-SA-(PEG-X)_{N},
dATP-PA-SS-PEG-biotin-SA-(PEG-X)_{N},
dGTP-PA-SS-PEG-biotin-SA-(PEG-X)_{N},
Modifications with SA-PE
dUTP-AA-SS-PEG-biotin-SA-PE
dCTP-PA-SS-PEG-biotin-SA-PE
dATP-PA-SS-PEG-biotin-SA-PE
dGTP-PA-SS-PEG-biotin-SA-PE

These compounds are accepted as substrates by polymerases (e.g., Klenow exo-minus polymerase, Sequenase, Vent exo-minus polymerase, Taq polymerase, Pwo polymerase, reverse transcriptase (M-MLV, (Promega), ImProm II™ (Promega)) and can be used in a sequencing reaction.

The mixture resulting from the synthesis, for instance, dATP-PA-SS-PEG-biotin-SA-(PEG-X)n and SA-(PEG-X)n can be used as a whole in the sequencing reaction.

Such modified nucleotide analogs comprise a cleavable linker, a macromolecular sterically demanding ligand (modified streptavidin molecule) and a marker, wherein the marker can consist of several dyes with low molecular weight (e.g., Cy3, FITC, Cy5) or a macromolecular marker like PE (phycoerhytrin). The possibility of binding different dyes to the streptavidin or its modifications (e.g., PEG-modified Streptavidin or SA-PE-conjugate) allows for different color codings for individual modified nuc-macromolecules.

Such modified nuc-macromolecules (nucleotide analogs) represent examples for reversible terminators with macromolecular sterically demanding ligands and can be used in sequencing methods like (WO02088382). Reversible terminators with termination efficiencies comprising the ranges from 80-100% and 90-100% are preferred for sequencing methods. Especially preferred are reversible terminators with terminating efficiencies in the ranges between 95-100%, 97-100% and 99-100%. After such a modified nuc-macromolecule has been incorporated, no other modified nuc-macromolecule can be incorporated. After the cleavage of the linker with reducing agent (e.g., TCEP) and the blockade of the mercapto group (e.g., with iodacetamide), another complementary modified nuc-macromolecule can be incorporated, likewise leading to a reversible stop.

### Example 14

The enzymatic incorporation reactions were carried out under conditions usually used for the incorporation reactions of modified nuc-macromolecules. For instance, the following conditions can be used:

Buffer solutions:
Tris-HCl (20 mM-100 mM), pH 7-8.5
(phosphate, MOPS, HEPES, acetate, and borate-buffer can also be used as buffers)
MgCl₂ e.g., 1.5 to 10 mM (or also Mn 0.2-1 mM)
NaCl 20 to 100 mM
Glycerol approximately 10-30%
Primers (oligonucleotides) with a length of 17 to 50 nucleotides which have a sufficient specific hybridization to the template.
Concentration approx. 0.02 to 2 μmol/l
Templates (PCR products, oligonucleotides)
DNA polymerases (Klenow fragment, Taq polymerase, Vent polymerase, Vent exo-minus polymerase, Deep Vent exo-minus polymerase, Pwo polymerase, Sequenase II, reverse transcriptases, AMV, M-MLV, RAV, HIV, ImProm II™ reverse transcriptase)
modified nuc-macromolecules used in concentrations mainly between 0.1 μmol/l to 50 μmol/l.

Microtiter plates, beads (e.g., streptavidin-coated polystyrene beads or paramagnetic particles based on dextran, e.g., from Promega) or DNA chips from various manufacturers are suitable for the reactions on the solid phase. The fixation of the nucleic acids on the solid phases takes place through affinity coupling or covalent coupling, depending on the experiment. The detection is performed according to the marker used: e.g., fluorescence or enzymatic color development. For instance, gel electrophoresis, gel filtration, ultrafiltration and affinity isolation can be used as separation media and methods.

Enzymatic reactions were carried out for approx. 2 to 60 min at RT to 60° C.

The cleavage reaction of the disulfide bond was carried out, for example, under the following conditions:
50 mmol/l borate buffer, pH 8.5-9.0, with 50 mmol/l NaCl
TCEP, beta-mercaptoethanol or DTT were used as reducing substances.
The reaction time can vary between 5 and 60 min at RT.

### Example 15

### Reversible Termination Using dCTP-PA-SS-(PEG)₈-Biotin-SA-Cy3

Demonstration of the reversible termination using the synthesis of a homopolymer region in an artificial sequence as an example. Detection was accomplished by measuring the fluorescence of the signals from modified nuc-macromolecules after a gel electrophoresis of extended primers.

### Materials

### Solutions:

Buffer 1: 50 mmol/l Tris HCl, pH 8.5; 50 mmol/l NaCl, 5 mmol/l MgCl₂, glycerol 10% v/v
Buffer 2: borate buffer 50 mmol/l pH 9.0; 100 mmol/l NaCl, 5 mmol/l MgCl₂

### Nucleotides and Nucleotide Analogs:

dATP (was purchased from Roth) and diluted to a solution of 1 mmol/l.

dCTP-PA-SS-(PEG)₈-biotin-SA-Cy3 (hereinafter called dC-analog).

In this example, dC-analog (aqueous solution, 100 μmol/l) is a component of a mixture. The mixture was obtained as described in example 11, and dC-analog was not separated from unconjugated SA-Cy3.

Solid phase: Streptavidin MagneSphere paramagnetic particles (cat. No. Z5481) Promega can be isolated from the solution with the help of a magnet (see manufacturer's instructions). The washing of the solid phase was carried out by repeated exchange of a solution (single volume of the solution: 200 μl).

For the sake of simplifying the description, the following will be called “solid phase”: beads themselves and all elements fixed to them, e.g., nucleic acids, nucleotides etc. During the cyclic reaction, aliquots were taken from the main reaction mixture at different points in time (as indicated in the text).

Polymerase: Vent exo-minus polymerase (New England Biolabs), is designated as polymerase.

### Nucleic Acids:

Artificial Oligonucleotides with Following Sequences:

Oligonucleotide-1: biotin-(T)₄₈
Oligonucleotide -2: (template)
5′(A)₅₀TCCCGTTTCGTCTCGTTCCGCAGGGTCCTATAGTGAGTCGTAT
TA 3′
Oligonucleotide -3: (Primer)
5′TAATACGACTCACTATAGG 3′

All oligonucleotides were purchased from MWG Biotech, Germany.

### General Reaction Conditions:

The primer-extension reaction was carried out at 37° C. in buffer 1 for 15 min. Under these conditions, over 95% of all extendable primer-template complexes were extended using the indicated polymerase- and nucleotide concentrations in a cycle of 15 min.

### Preparation of the Solid Phase for the Cyclic Reactions:

Combine three vials of paramagnetic particles and wash in buffer 1 and then dissolve the solid phase as a suspension in 200 μl of buffer 1. Next, bind oligonucleotide-1 to the solid phase: add a solution with oligonucleotide-1 (7 μl 100 μM in water) to the solid phase and agitate at RT for 10 min. Then wash the solid phase with buffer 1. Add a solution with oligonucleotide-2 (5 μl 100 μM in water) and a solution with oligonucleotide-3 (5 μl 100 μM in water) to the solid phase together and incubate at 37° C. for 10 min. Next, wash the solid phase in buffer 1. Such a solid phase can be used in enzymatic reactions; it comprises a template (oligonucleotide-2) and a primer (oligonucleotide-3).

Take an aliquot of this solid phase (15% of the total quantity of the solid phase) (specimen 1). Add buffer 1 to this aliquot to attain a total volume of 90 μl. After that, add a mixture with dC-analog (10 μl 100 μmol/l nucleotide in the buffer 1) and incubate at 37° C. for 15 min. Next, wash the solid phase with buffer 1 several times. This specimen 1 serves as a control for an unspecific binding of dC-analog to the solid phase.

### Cyclic Reactions:

All incubation steps with polymerases and nucleotide analogs were carried out in a volume of 100 μl in buffer 1.

### 1. Binding of the Polymerase:

Add a solution of polymerase (5 μl in the manufacturer's buffer) to the solid phase and incubate at RT for 5 min. Then wash the solid phase with buffer 1. The polymerases remain bonded to the nucleic acids. Suspend the solid phase in 100 μl of buffer 1.

### 2. Addition of Nucleotides and Nucleotide Analogs:

Add a solution of dATP (5 μl 1 mmol/l) and dC-analog (10 μl 100 μmol/l) in buffer 1 to the solid phase. Incubate the solid phase with the above components at 37° C. for 15 min. Next, wash the solid phase with buffer 1 and take an aliquot (specimen 2). Specimen 2 comprises dC-analogs coupled to the primer_{(N+2)}. Only a single dC-analog is incorporated, since the macromolecular sterically demanding ligand prevents further progress of the synthesis.

### 3. Cleaving-Off of the Macromolecular Sterically Demanding Ligand and Modification of the Linker Residue

Next, wash the solid phase with buffer 2. Then, first incubate the solid phase with a solution of DTT (100 μl 50 mmol/l in buffer 2) for 10 min at RT and afterwards with a solution of iodacetamide (100 μl 0.2 mol/l in buffer 2) at RT for 10 min. Take another aliquot (specimen 3). Specimen 3 contains the primer_{(N+2)} with the linker residue on the incorporated dC-analog, and the sterically demanding ligand has been cleaved off and the liberated mercapto group has been modified.

### 4. Binding of the Polymerase:

Next, wash the solid phase with buffer 1 and repeat step 1: add another 5 μl of Vent exo-minus polymerase to the manufacturer's buffer. After 5 min at RT, wash the solid phase with buffer 1 several times.

### 5. Addition of Nucleotide Analogs:

Add dC-analog (10 μl 100 μmol/l) in buffer 1 to the solid phase. Incubate the solid phase at 37° C. for 15 min. Next, wash the solid phase with buffer 1 and take an aliquot (specimen 4). Specimen 4 contains the primer_{(N+3)} with the coupled dC-analog. Another dC-analog has now been incorporated.

### 6. Cleaving-Off of the Macromolecular Sterically Demanding Ligand and Modification of the Linker Residue (See Above).

Take an aliquot (specimen 5). Specimen 5 contains the primer_{(N+3)} with the linker residue on the incorporated dC-analog, and the sterically demanding ligand has been cleaved off and the liberated mercapto group has been modified.

### 7. Binding of the Polymerase:

Next, wash the solid phase with buffer 1 and repeat step 1: add another 5 μl of Vent exo-minus polymerase in the manufacturer's buffer. After 5 min at RT, wash the solid phase with buffer 1 several times.

### 8. Addition of Nucleotide Analogs:

Add dC-Analog (10 μl 100 μmol/l) in buffer 1 to the solid phase. Incubate the solid phase at 37° C. for 15 min. Then wash the solid phase with buffer 1 and take an aliquot (specimen 6). Specimen 6 contains dC-analogs coupled to the primer_{(N+4)}. Another dC-Analog has now been incorporated.

The electrophoretic separation of specimens 1 to 6 was performed using polyacrylamide gel (6% w/v) in 50 mmol/l Tris-HCl, pH 8.5, at 200 V on Miniprotean equipment (Biorad, Germany). The electrophoresis was carried out at 60° C.

### Legend for FIG. 20:

Line 1: ladder (dC-analog (upper band) and oligonucleotide-3, labeled with Cy3 dye at 3′ ends (lower band)
Line 2: specimen 1 (control for an unspecific binding of dC-analog to the solid phase)
Line 3: specimen 2 (incorporation of the 1^{st} dC-analog)
Line 4: specimen 3 (cleaving-off of sterically demanding ligands with marker)
Line 5: specimen 4 (incorporation of the 2^{nd} dC-analog)
Line 6: specimen 5 (cleaving-off of sterically demanding ligands with marker)
Line 7: specimen 6 (incorporation of the 3^{rd} dC-analog)

This example demonstrates the reversible termination of the synthesis by means of modified nuc-macromolecules according to the invention with a macromolecular sterically demanding ligand.

Other nucleotides can also be modified in a manner similar to that for dCTP-AA-SS-(PEG)₈-biotin-SA-Cy3 and dCTP-AA-SS-(PEG)₈-biotin-SA-PE (see example 11). The following compounds represent examples of reversible terminators with macromolecular sterically demanding ligands:

dUTP-AA-SS-(PEG)₈-biotin-SA
dCTP-PA-SS-(PEG)₈-biotin-SA
dATP-PA-SS-(PEG)₈-biotin-SA
dGTP-PA-SS-(PEG)₈-biotin-SA
dUTP-AA-SS-(PEG)₈-biotin-SA-PE
dCTP-PA-SS-(PEG)₈-biotin-SA-PE
dATP-PA-SS-(PEG)₈-biotin-SA-PE
dGTP-PA-SS-(PEG)₈-biotin-SA-PE

These compounds are accepted as substrate by polymerases (e.g., Klenow-Exo minus Polymerase, Sequenase, Vent Exo minus Polymerase, Taq-Polymerase, Pwo Polymerase) and can be used as a reversible terminators in sequencing reactions.

### Example 16

### Examples of Applications of Modified Nuc-Macromolecules in Sequencing at the Single-Molecule Level

Several patent applications which describe different applications and embodiments of the sequencing by synthesis (WO02088382, DE 102004025746, DE 10120798, DE 10246005, EP1692312, EP1766090, WO2007100637) have been published. These documents are cited here and incorporated in full scope as citations (within the meaning of “incorporated by reference” in full scope). In the following, some exemplary embodiments are described in which modified nuc-macromolecules with sterically demanding macromolecular ligands can be used.

These methods relate to the design of extendable primer-template complexes on a solid phase, wherein the primers are extended in cyclic steps and signals are detected by incorporated modified nuc-macromolecules. The solid phase can be in the form of a planar surface or in the form of nano- or microparticles (e.g., beads). The beads also can be distributed on a planar surface so that a two-dimensional array results. Such solid phases are preferably components of kits for the sequencing.

The individual extendable primer-template complexes (a template molecule binds one primer-molecule) are preferably bonded to the solid phase in a density, which allows for optical assignment of incorporation events (e.g., fluorescence signals from incorporated modified nuc-macromolecules) to individual primer-template complexes (WO02088382, DE 102004025746). For instance, fluorescence microscopes can be used as detecting devices (DE 10246005). The solid phase prepared in this manner allows for the observation of cyclic reactions on the solid phase at the level of single molecules (e.g., DE 102004025746). Accordingly, the surface is scanned and the positions of individual signals on the surface are detected, so that every extendable primer-template complex is assigned to a specific position on the surface with coordinates (X, Y). During repeated scan-cycles, signals can be assigned to the respective primer-template complexes.

Different nucleic acid chains can be used as material: Both pre-selected DNA sequences (e.g., isolated PCR fragments, genome fragments cloned in YAC-, PAC-, or BAC vectors (R. Anand et al. NAR 1989 v. 17 p. 3425, H. Shizuya et al. PNAS 1992 v. 89 p. 8794, “Construction of bacterial artificial chromosome libraries using the modified PAC system” in “Current Protocols in Human genetics” 1996 John Wiley & Sons Inc.) and non-preselected DNA (e.g., genomic DNA, cDNA mixtures, PCR fragments mixtures, mRNA mixtures, oligonucleotide libraries). Applying a pre-selection, it is possible to limit the focus only to relevant information, as for example sequence segments from a genome or populations in genetic products, and filter out large quantities of genetic information, thereby limiting the number of the sequences to be analyzed.

The object of the material preparation is to obtain bound single-strand NACFs with a length of preferably 50-1000 NTs, a single primer binding site and a hybridised primer (bound NACF primer complexes). In particular, highly variable structures can be derived from this general structure. To improve clarity, a few examples now follow, with the methods cited being usable individually or in combination.

Preparation of short nucleid acid fragment (50-1000 NTs) (fragmentation step).

It is important that fragmentation of the NACs takes place in such a way that fragments are obtained that represent partial sequences of the overall sequences. This is achieved by methods in which fragments of differing length are formed as cleavage products in random distribution.

According to the invention, the production of the nucleic acid chain fragments (NACFs) can take place by several methods, for example by fragmentation of the starting material with ultrasound or by endonucleases (“Molecular cloning” 1989 J. Sambrook et al. Cold Spring Harbor Laborotary Press), such as for example by non-specific endonuclease mixtures. According to the invention, ultrasound fragmentation is preferred. The conditions can be adjusted in such a way that fragments with a mean length of 100 by to 1 kb are formed. These fragments are then filled up at their ends by the Klenow fragment (E. coli polymerase I) or by T4-DNA polymerase (“Molecular cloning” 1989 J. Sambrook et al. Cold Spring Harbor Laborotary Press).

In addition, complementary short NACFs can be synthesised from a long NAC by using randomised primer. This method is particularly preferred in the analysis of the gene sequences, Single-strand DNA fragments are in this connection formed at the mRNA with randomised primers and a reverse transcriptase (Zhang-J et al. Biochem. J. 1999 v. 337 p. 231, Ledbetter et al. J. Biol. Chem. 1994 v. 269 p. 31544, Kolls et al. Anal. Biochem. 1993 v. 208 p. 264, Decraene et al. Biotechniques 1999 v. 27 p. 962).

### Introduction of a Primer Binding Site in the NACF

The primer binding site (PBS) is a sequence section that is intended to allow selective binding of the primer to the NACF.

In one embodiment, the primer binding sites may be different, so that several different primers must be used. In this case, particular sequence sections of the total sequence can serve as natural PBSs for specific primers. This embodiment is particularly suitable for the investigation of SNP sites already known.

In another embodiment, it is favourable, for the purposes of simplifying the analysis, if a uniform primer binding site is present in all NACFs. According to a preferred embodiment of the invention, the primer binding sites are therefore additionally introduced in the NACFs. Primers with a uniform structure can in this way be used for the reaction.

This embodiment is described in detail below.

The composition of the primer binding site is not restricted. Its length is preferably between 20 and 50 NTs. The primer binding site may bear a functional group to immobilise the NACF. This functional group may be, for example, a biotin group.

As an example of the introduction of a uniform primer binding site, ligation and nucleotide tailing on DNA fragments are described below.

### a) Ligation:

In this process, a double-stranded oligonucleotide complex with a primer binding site is used. This is ligated with commercially available ligases to the DNA fragments (“Molecular cloning” 1989 J. Sambrook et al. Cold Spring Harbor Laborotary Press). It is important that only a single primer binding site is ligated to the DNA fragment. This is achieved for example by a modification of one side of the oligonucleotide complex on both strands. The modifying groups on the oligonucleotide complex can serve for immobilisation. The synthesis and modification of such an oligonucleotide complex can be performed in accordance with standardised instructions. DNA-Synthesizer 380 A Applied Biosystems can be used for example for the synthesis. Oligonucleotides with a specific composition with or without modifications are, however, also commercially available as application synthesis, for example from MWG-Biotech GmbH, Germany.

### b) Nucleotide Tailing:

Instead of ligation with an oligonucleotide, several (e.g. between 10 and 20) nucleoside monophosphates can be coupled to the 3′ end of an ss-DNA fragment with a terminal deoxynucleotidyl-transferase (“Molecular cloning” 1989 J. Sambrook et al. Cold Spring Harbor Laborotary Press, “Method in Enzymology” 1999 v. 303, pp. 37-38), e.g. several guanosine monophosphates (called (G)n-tailing). The fragment formed is used to bind the primer, in this example a (C)n primer.

### Single-Strand Preparation

Single-strand NACFs are needed for the sequencing reaction. If the starting material is present in double-stranded form, there are several ways of producing a single-stranded form from double-stranded DNA (e.g. heat denaturation or alkali denaturation) (“Molecular cloning” 1989 J. Sambrook et al. Cold Spring Harbor Laborotary Press).

### Primer for the Sequencing Reaction

This has the function of enabling start-up at a single location in the NACF. It binds to the primer binding site in the NACF. The composition and length of the primer are not restricted. Apart from the start function, the primer can also assume other functions, such as for example establishing a link to the reaction surface. Primers should be adapted to the length and composition of the primer binding site in such a way that the primer enables start-up of the sequencing reaction with the respective polymerase.

When different primer binding sites, for example primer binding sites naturally occurring in the original overall sequence, are used, the primers that are sequence-specific for the respective primer binding site are used. In this case, a primer mixture is used for the sequencing.

In the case of a uniform primer binding site, for example a primer binding site coupled to the NACFs by ligation, a uniform primer is used.

The length of the primer is preferably between 6 and 100 NTs, optimally between 15 and 30 NTs. The primer can bear a function group that serves to immobilise the NACF, for example such a function group is a biotin group (see section on immobilisation). It is not to disturb the sequencing. The synthesis of such a primer may for example be performed with the DNA-Synthesizer 380 A Applied Biosystems or alternatively conducted as application synthesis by a commercial supplier, for example MWG-Biotech GmbH, Germany).

In one embodiment, a primer is attachned to the surface, as described in this application.

The primer or the primer mixture is incubated with NACFs under hybridisation conditions that cause it to bind selectively to the primer binding site. This primer hybridisation (annealing) can take place before (1), during (2) or after (3) the binding of the NACFs to the surface. Optimisation of the hybridisation conditions depends on the precise structure of the primer binding site and the primer and can be calculated in accordance with Rychlik et al. NAR 1990 v. 18 p. 6409. These hybridisation conditions are in what follows designated as standardised hybridisation conditions.

If a primer binding site of known structure that is common to all NACFs is introduced for example by ligation, primers of uniform structure can be used. The primer binding site may bear a functional group at its 3′ end, which serves for example for immobilisation. This group is for example a biotin group. The primer has a structure complementary to the primary binding site.

Fixing of NACF primer complexes to the surface (binding or immobilisation of NACFs).

The object of the fixing (immobilisation) is to fix NACF primer complexes on a suitable planar surface in such a way that a cyclical enzymatic sequencing reaction can take place. This may for example take place by binding of the primer (see above) or the NACF to the surface.

The sequence of the steps in the fixing of NACF primer complexes may be variable:
1) The NACF primer complexes can first of all be formed in a solution by hybridisation (annealing) and then bound to the surface.
2) Primers can first of all be bound on a surface and NACFs then hybridised to the bound primers, with NACF primer complexes being formed (NACFs indirectly bound to the surface).
3) The NACFs can first of all be bound to the surface (NACFs directly bound to the surface) and, in the next step, the primers are hybridised to those bound NACFs, with NACF primer complexes being formed.

Immobilisation of the NACFs to the surface can therefore take place via direct or indirect binding.

If fixing of the NACF primer complexes on the surface takes place via the NACFs, this may for example take place via binding of the NACFs to one of the two chain ends. This can be achieved by corresponding covalent, affine or other bonds. Many examples of the immobilisation of nucleic acids are known (McGall et al. U.S. Pat. No. 5,412,087, Nikiforov et al. U.S. Pat. No. 5,610,287, Barrett et al. U.S. Pat. No. 5,482,867, Mirzabekov et al. U.S. Pat. No. 5,981,734, “Microarray biochip technology” 2000 M. Schena Eaton Publishing, “DNA Microarrays” 1999 M. Schena Oxford University Press, Rasmussen et al. Analytical Biochemistry v. 198, p. 138, Allemand et al. Biophysical Journal 1997, v. 73, p. 2064, Trabesinger et al. Analytical Chemistry 1999, v. 71, p. 279, Osborne et al. Analytical Chemistry 2000, v. 72, p. 3678, Timofeev et al. Nucleic Acid Research (NAR) 1996, v. 24 p. 3142, Ghosh et al. NAR 1987 v. 15 p. 5353, Gingeras et al. NAR 1987 v. 15 p. 5373, Maskos et al. NAR 1992 v. 20 p. 1679).

A cyclic reaction is started after the preparation of primer-template comlexes, wherein modified nuc-macromolecules are used. The reaction takes place in several steps:
Incubation of at least one type of modified nuc-macromolecule, in accordance with aspects 1 to 25, together with one type of polymerase, in accordance with aspect 31, with NAC primer complexes, prepared in steps (a) and (b), under such conditions as allow for the incorporation of complementary modified nuc-macromolecules, whereby each type of modified nuc-macromolecule has characteristic labeling.
Removal of the non-incorporated modified nuc-macromolecules from the NAC Primer complexes.
Detection of the signals from the modified nuc-macromolecules incorporated into the NAC Primer complexes.
Removal of the linker component and the sterically demanding ligand and the marker component from the modified nuc-macromolecules incorporated into the NAC Primer complexes.
Washing the NAC Primer complexes.

These steps can be repeated several times to allow to reconstruct a complementary sequence of the template from the order of the detected signals from incorporated nucleotide analogs. This iteration can be done, for instance, 1 to 2, 2 to 5, 5 to 10, 10 to 20, 20 to 30, 30 to 50, 50 to 100, 100 to 2000 times.

The reaction times in a cycle are chosen in such a way that the polymerases can incorporate a labeled modified nuc-macromolecule in more than 50% of the NACFs involved in the sequencing reaction (extendable NACF primer complexes) in a cycle, preferably in more than 90%.

A color coding scheme for modified nuc-macromolecules can be different. A cycle can be performed with:
a) four differently labelled modified nuc-macromolecules
b) two differently labelled modified nuc-macromolecules
c) one labelled modified nuc-macromolecule
d) two differently labelled modified nuc-macromolecules and two unlabelled modified nuc-macromolecules
   i.e.

a) All 4 modified nuc-macromolecules can be labelled with different dyes and all 4 can be used simultaneously in the reaction. The sequencing of a nucleic acid chain with a minimum number of cycles is achieved in this case. However, this variant of the invention makes great demands of the detection system: 4 different dyes must be identified in each cycle.

b) To simplify detection, labelling with two dyes can be chosen. Here, 2 pairs of modified nuc-macromolecules are formed that are each differently labelled, e.g. A and G bear the labelling “X”, C and U bear the labelling “Y”. In the reaction in a cycle (n), 2 differently labelled nucleotide analogs are used at the same time, e.g. C* in combination with A*, and U* and G* are then added in the following cycle (n+1).

c) only a single dye can be used to label all 4 modified nuc-macromolecules and only one modified nuc-macromolecules can be used per cycle.

Cycles, in which modified nuc-macromolecules are used (within the meaning of this application), can alternate with cycles in which unmodified nucleotides are used.

For instance, first carry out 5 to 500 cyclic steps with modified nuc-macromolecules, then 1 to 500 cyclic steps with unmodified nucleotides (e.g., with naturally occurring nucleotides, dATP, dGTP, dTTP, dCTP or with their analogs, like dUTP, dITP or with other nucleotide analogs, which have no macromolecular sterically demanding ligand), and then again follow with 10 to 500 steps with modified nuc-macromolecules etc.

The order of the added nucleotides and the cycle number can vary. Possible combinations for the use of modified nuc-macromolecules and the cycle numbers were already discussed above (see color-coding schemes). The unmodified nucleotides can likewise be added ether individually, or in pairs or in threes with a suitable polymerase under conditions that allow for the extension of primer-template complexes. This limited feeding of substrate allows for a stepwise primer extension. Several cycles, each with a different composition of natural nucleotides, can be carried out, for instance, dATP, dGTP and dCTP can be added in one cycle, dATP, dGTP and dTTP can be added in another cycle, and dCTP, dGTP and dTTP can be added in still another cycle. Further combinations will be obvious. Also all 4 natural nucleotides can be used, assuming that one of them is added in a limited or strongly reduced concentration.

The number of the changes between the individual cycles with modified and unmodified nucleotides comprises ranges between 2 to 500.

The use of combinations comprising steps with modified nuc-macromolecules and steps with unmodified nucleotides provides the possibility of skipping longer sequences of the template without consuming reversible terminating nucleotides. This can be advantageous if, for technical reasons (e.g. due to high specificity), a primer is hybridized relatively far from the region of the template to be sequenced. Another example of a possible application of this embodiment of the sequencing method is a screening of mRNA- or cDNA molecules for completeness: an identification of exons can be accomplished by sequencing relatively short fragments, and sequences irrelevant to analysis can be skipped by incorporating natural nucleotides.

Altogether, the number of the cyclic steps with reversible terminating nucleotide analogs with a macromolecular steric obstacle comprises a range between 2 and 10000.

In a further embodiment of the processes, the incorporation reaction of nuc-macromolecules occurs simultaneously on a population of different nucleic acid molecules attached to a solid phase, whereby the said nucleic acid molecules are attached to the solid phase in a random arrangement (Tcherkassov WO 02088382). In this process, sequences are determined for individual nucleic acid chain molecules. The primer nucleic acid complexes taking part in the enzymatic reaction are attached in such a density as allows for the detection of signals from single modified nuc-macromolecules coupled to a single nucleic acid molecule, but the density of the attached primer or nucleic acid can be substantially higher. For instance, the density of the primer nucleic acid complexes taking part in the incorporation reaction ranges between 1 to 10 complex per 10 μm², 1 to 10 complex per 100 μm², 1 to 10 complex on 1000 μm², 1 to 10 complex per 10,000 μm².

Examples of the attachment of nucleic acids to the solid phase in such a density as allows for analyses on single molecules are shown in WO0157248, U.S. Patent No. 2003064398, U.S. Patent No. 2003013101 and WO 02088382. Suitable equipment for detection is described in WO 03031947.

The number of single nucleic acid molecules to be analyzed ranges, for instance, between 1000 and 100,000, 10,000 to 1,000,000, 100,000 to 100,000,000 molecules. The marker component or its individual constituents with or without a linker component of the modified nuc-macromolecule are cleaved from the nuc-component during or after the incorporation reaction.

The said method for the parallel sequence analysis of nucleic acid sequences (nucleic acid chains, NAC) comprises the following steps, in which:
Fragments (NACFs) of single-strand NACs with a length of approximately 50-1000 nucleotides are produced that may represent overlapping partial sequences of a whole sequence.
The NACFs are bound in a random arrangement using one uniform or several different primers in the form of NACF primer complexes on a reaction surface, whereby the density of NACF primer complexes bound to the surface allows for optical detection of signals from individual incorporated modified nuc-macromolecules.
A cyclical synthesis reaction of the complementary strand of the NACFs is performed using one or more polymerases by:
   a) adding to the NACF primer complexes bound to the surface a solution comprising one or more polymerases and one to four modified nuc-macromolecules that have a marker component labeled with fluorescent dyes, with concomitant use of at least two modified nuc-macromolecules with dyes coupled to the marker component, being chosen in such a way that the modified nuc-macromolecules used can be distinguished from one another by the measurement of different fluorescent signals, with the modified nuc-macromolecules comprising a macromolecular sterically demanding ligand, wherein linker component and marker component and macromolecular sterically demanding ligand being removable,
   b) incubating the stationary phase obtained in step a) under conditions suitable for extending the complementary strands, with the complementary strands being extended in each case by one modified nuc-macromolecule,
   c) washing the stationary phase obtained in step b) under conditions suitable for the removal of modified nuc-macromolecules not incorporated in a complementary strand,
   d) detecting the single modified nuc-macromolecules incorporated in complementary strands by measuring the signal characteristic of the respective fluorescent dye, with the relative position of the individual fluorescent signals on the reaction surface being determined at the same time,
   e) cleaving off the linker component and marker component of the nuc-components added to the complementary strand in order to produce unlabeled (NTs or) NACFs,
   f) washing the stationary phase obtained in step e) under conditions suitable for the removal of the marker component,
   repeating steps a) to f), where appropriate several times,
with the relative position of individual NACF primer complexes on the reaction surface and the sequence of these NACFs being determined by specific assignment of the fluorescent signals detected in step d) in successive cycles in the respective positions to the modified nuc-macromolecules.

### Example 17

### Preparation of the Reaction Surface

The surface and reaction surface are for the present purposes to be conceived of as identical concepts, except where another meaning is explicitly indicated. The surface of a solid phase of any material serves as reaction surface. This material is preferably inert to enzymatic reactions and causes no disturbances in detection. Silicone, glass, ceramics, plastic (e.g. polycarbonates or polystyrenes), metal (gold, silver or aluminium) or any other material that meets these functional requirements can be used. The surface is preferably not deformable since distortion of the signals in the case of repeated detection may otherwise be expected.

The various cycle steps require exchange of the various reaction solutions over the surface. The reaction surface preferably forms part of a reaction vessel. The reaction vessel in turn preferably forms part of reaction equipment with a flow device. The flow device allows for exchange of the solutions in the reaction vessel. The exchange can take place with a pump device controlled by a computer or manually. It is important in this context that the surface does not dry out. The volume of the reaction vessel is preferably less than 50 μl. Ideally, its volume is less than 5 μl.

If fixing of the NACF primer complexes on the surface takes place via the NACFs, this may for example take place via binding of the NACFs to one of the two chain ends. This can be achieved by corresponding covalent, affine or other bonds. Many examples of the immobilisation of nucleic acids are known (McGall et al. U.S. Pat. No. 5,412,087, Nikiforov et al. U.S. Pat. No. 5,610,287, Barrett et al. U.S. Pat. No. 5,482,867, Mirzabekov et al. U.S. Pat. No. 5,981,734, “Microarray biochip technology” 2000 M. Schena Eaton Publishing, “DNA Microarrays” 1999 M. Schena Oxford University Press, Rasmussen et al. Analytical Biochemistry v. 198, p. 138, Allemand et al. Biophysical Journal 1997, v. 73, p. 2064, Trabesinger et al. Analytical Chemistry 1999, v. 71, p. 279, Osborne et al. Analytical Chemistry 2000, v. 72, p. 3678, Timofeev et al. Nucleic Acid Research (NAR) 1996, v. 24 p. 3142, Ghosh et al. NAR 1987 v. 15 p. 5353, Gingeras et al. NAR 1987 v. 15 p. 5373, Maskos et al. NAR 1992 v. 20 p. 1679). Fixing may also be achieved by non-specific binding, such as for example by drying-out of the sample containing the NACFs on the planar surface.

The NACFs are bound on the surface, for example in a density of 10-100 NACFs per 100 μm², 100-10,000 per 100 μm² or 10,000-1,000,000 per 100 μm².

The density of extendable NACF primer complexes needed for detection is approximately 1-100 per 100 μm². It may be achieved before, during or after hybridisation of the primers against the NACF.

By way of example, several methods for binding NACF primer complexes are described in more detail below: in one embodiment, immobilisation of the NACFs takes place via biotin-avidin or biotin-streptavidin binding. Avidin or streptavidin is in this connection covalently bound on the surface, the 5′ end of the primer contains biotin. Following hybridisation of the labelled primers with the NACFs (in solution), these are fixed on the surface coated with avidin/streptavidin. The concentration of the hybridisation products labelled with biotin and the duration of incubation of this solution with the surface is chosen in such a way that a density suitable for sequencing is achieved by this stage.

In another preferred embodiment, the primers suitable for the sequencing reaction are fixed on the surface by suitable methods before the sequencing reaction (see above). The single-strand NACFs each with a primer binding site per NACF are thereby incubated under hybridisation conditions (annealing). In this connection, they bind to the fixed primers and are thereby bound (indirect binding), with primer NACF complexes being formed. The concentration of the single-strand NACFs and the hybridisation conditions are chosen in such a way that an immobilisation density suitable for sequencing of 10-100 extendable NACF primer complexes per 100 μm² is achieved. After the hybridisation, unbound NACFs are removed by a washing step. In this embodiment, a surface with a high primer density is preferred, for example approximately 1,000,000 primers per 100 μm² or even higher as the desired density of NACF primer complexes is achieved more rapidly, with the NACFs binding only to part of the primers.

In another embodiment, the NACFs are directly bound to the surface (see above) and then incubated with primers under hybridisation conditions. At a density of approximately 1 to 100 NACFs per 100 μm², it will be attempted to provide all available NACFs with a primer and make them available for the sequencing reaction. This can be achieved for example by a high primer concentration, for example 1 to 100 mmol/l. In the case of a higher density of the fixed NACFs on the surface, for example 10,000 to 1,000,000 per 100 μm², the density of the NACF primer complexes that is required for optical detection can be achieved during primer hybridisation. The hybridisation conditions (for example, temperature, time, buffer, primer concentration) are in this connection to be chosen in such a way that the primers bind only to part of the immobilised NACFs.

If a gel-like solid phase (surface of a gel) is used, this gel may be for example an agarose or polyacrylamide gel (DE 101 49 786). Owing to binding of the NACF primer complexes on the surface, detection of the fluorescent signals of incorporated incorporated nucleotide analogs is possible. The gel is preferably attached on a solid surface. This solid surface may be silicone, glass, ceramics, plastic (e.g. polycarbonates or polystyrenes), metal (gold, silver or aluminium) or any other material. Examples for preparation of the support for the solid phase see DE 102004025746.

Further examples for manufacturing of the reaction surface are known (US2005244863, EP1105529).

The surface may be produced as a continuous surface or as a discontinuous surface composed of individual small constituents (e.g. primer-template-complexes cab be attached to agarose beads or dextran beads). For instance, the density of beads on the surface ranges between 1 and 10 pro 100 μm², 10 and 100 pro 100 μm², 100 to 10.000 pro 100 μm², 10.000 to 1.000.000 pro 100 μm².

The reaction surface must be large enough to be able to immobilise the necessary number of NACFs with the corresponding density. The reaction surface should preferably be no greater than 20 cm². If a surface of a solid phase (e.g. silicon or glass) is used for attachment, it can be produced according to DE 102004025745.

Detection can be performed according to WO02088382 or DE10246005.

### The Analysis of the Nucleic Acids can Address Different Issues:

(Using Example of Sequence Analysis with Four Identic Labelled Modified Nuc-Macromolecules).
3A. Reconstruction of the Original Sequences in Accordance with the Shotgun Principle
(“Automated DNA sequencing and analysis” p. 231 et seq. 1994 M. Adams et al. Academic Press, Huang et al, Genom Res. 1999 v. 9 p. 868, Huang Genomics 1996 v. 33 p. 21, Bonfield et al. NAR 1995 v. 23 p. 4992, Miller et al. 3. Comput. Biol. 1994 v. 1 p. 257). (This principle is suitable in particular for the analysis of new, unknown sequences).

### 3A-1 Sequencing of a Long Piece of DNA

The sequencing of long nucleic acid chains is to be described schematically in the following with the aid of the sequencing of a 1 Mb long piece of DNA. The sequencing is based on the Shotgun principle (“Automated DNA sequencing and analysis” p. 231 et seq. 1994 M. Adams et al. Academic Press, Huang et al. Genom Res. 1999 v. 9 p. 868, Huang Genomics 1996 v. 33 p. 21, Bonfield et al. NAR 1995 v. 23 p. 4992, Miller et al. J. Comput. Biol. 1994 v. 1 p. 257). The material to be analysed is prepared for the sequencing reaction by being broken down into fragments preferably 50 to 1000 bp in length. Each fragment is then provided with a primer binding site and a primer. This mixture of various DNA fragments is now fixed on a planar surface. The non-bound DNA fragments are removed by a washing step. The sequencing reaction is then performed on the entire reaction surface. To reconstruct a 1 Mb long DNA sequence, the sequences of NACFs should preferably be about 100 NTs long.
In all, around 10 to 100 times the quantity of raw sequences are needed to reconstruct the original sequence
The NACF sequences determined represent a population of overlapping partial sequences that can be combined into the overall sequence of the NAC with commercially available programs (“Automated DNA sequencing and analysis” p. 231 et seq. 1994 M. Adams et al. Academic Press, Huang et al. Genom Res. 1999 v. 9 p. 868, Huang Genomics 1996 v. 33 p. 21, Bonfield et al. NAR 1995 v. 23 p. 4992, Miller et al. J. Comput. Biol. 1994 v. 1 p. 257).
   3A-2 Sequencing of the Gene Products Based on the Example of cDNA Sequencing
In a preferred embodiment, several sequence can be analysed in a batch instead of one sequence. The original sequences can be reconstructed from the raw data obtained, for example by the Shotgun principle.
First of all, NACFs are produced. For example, mRNA can be converted into a double-stranded cDNA and this cDNA can be fragmented with ultrasound. These NACFs are then provided with a primer binding site, denatured, immobilised and hybridised with a primer. In this variant of the sample preparation, it should be noted that the cDNA molecules may represent incomplete mRNA sequences (Method in Enzymology 1999, v. 303, p. 19 and other articles in this volume, “cDNA library protocols” 1997 Humana Press).
Another option for the generation of single-stranded NACFs of mRNA consists in the reverse transcription of the mRNA with randomised primers. Many relatively short antisense DNA fragments are formed in this connection (Zhang-J et al. Biochem. J. 1999 v. 337 p. 231, Ledbetter et al. J. Biol. Chem. 1994 v. 269 p. 31544, Kolls et al. Anal. Biochem. 1993 v. 208 p. 264, Decraene et al. Biotechniques 1999 v. 27 p. 962). These fragments may then be provided with a primer binding site (see above). Further steps are in accordance with the processes described above. Complete mRNA sequences (from 5′ to the 3′ end) can be analysed by this method as the randomised primers bind over the entire length of the mRNA.
Immobilised NACFs are analysed with one of the aforementioned embodiments of the sequencing. The number of NACFs that must be analysed is calculated by the same principles as for a Shotgun reconstruction of a long sequence.
The original gene sequences are reconstructed from NACF sequences in accordance with the principles of the Shotgun method.
This method permits the simultaneous sequencing of many mRNAs without previous cloning.

### 3B. Analysis of Sequence Variants

Confirmation of a sequence already known or proof of variants of this sequence makes very much lesser demands of the length and redundancy of the NACF sequences determined. Sequence processing is also simpler in this case. The full sequence does not need to be reconstructed. Rather, the NACF sequences are assigned to the full sequence with the aid of a commercially available program and any non-conformities detected. Such a program may be based on, for example, the BLAST or FASTA algorithm (“Introduction to computational Biology” 1995 M. S. Waterman Chapman & Hall).
The sequence to be analysed is converted into NACFs by one of the aforementioned methods. These NACFs are sequenced by the method according to the invention, with both a uniform primer and a uniform primer binding site as well as different, sequence-specific primers and natural primer binding sites occurring in the overall sequence to be investigated, see example 5, being usable. The sequences of NACFs determined are then not combined in accordance with the Shotgun method but compared with the reference sequence and, in this way, their positions in the full sequence assigned. Genomic or cDNA sequences may be involved.
Unlike reconstruction by the Shotgun method, considerably less raw sequence data are needed for the analysis of a sequence variant. Thus, 5 to 10 times the raw sequence quantity may be sufficient for the restoration of a new variant of a full sequence. With the Schrotschuss method, 10 to 100 times the quantity of raw sequences is needed for restoration (“Automated DNA sequencing and analysis” p. 231 et seq. 1994 M. Adams et al. Academic Press, Huang et al, Genom Res. 1999 v. 9 p. 868, Huang Genomics 1996 v. 33 p. 21, Bonfield et al. NAR 1995 v. 23 p. 4992, Miller et al. J. Comput. Biol. 1994 v. 1 p. 257).
The length of the NACF sequences determined is to be sufficient for clear assignment to a specific position in the reference sequence; thus, for example, even sequences with a length of 20 NTs (e.g. comprising non-repetitive sections in the human genome) can be clearly identified. Longer sequences are required for comparative analysis of the repetitive sections, with the precise length of the sequences depending on the task. The length of the NACF sequences determined for the analysis of non-repetitive sections is preferably more than 20 NTs. For the analysis of repetitive sections, it is preferably more than 500 NTs.
The investigated whole length sequence can comprise segments with all nucleotides sequenced and segments which were synthesized by addition of non-labelled nucleotides in known combinations to allow a calculated primer extension to proceed.
The objectives in the sequencing of new variants of a previously known full sequence may be very different. A comparison of the newly determined sequence with the known full sequence/reference sequence is mostly sought. The two sequences may in this connection originate from species that are widely different in evolutionary terms. Various parameters of the composition of these two sequences may be compared. The following serve as examples of such analysis: mutation or polymorphism analyses and the analysis of alternatively spliced gene products.
A comparison of the sequence to be investigated with a reference sequence without prior reconstruction of the sequence to be analysed is to be considered schematically and on an exemplary basis in what follows. Such a comparison may, for example, be used for the mutation or SNP analysis.

### 3B-1

A long sequence to be analysed, e.g. 1 Mb, is divided into NACFs by one of the aforementioned method. These NACFs are sequenced using uniform primers by the method according to the invention. The sequences of each individual NACF that are determined are compared directly with the reference sequence. The reference sequence serves in this connection as the basis for the assignment of NACF sequences determined, so that expensive reconstruction by the Shotgun method is dispensed with. The length of the NACF sequences determined in the analysis of non-repetitive sections is preferably more than 20 NTs. For analysis of the repetitive sections, it is preferably more than 500 NTs. The number of NACFs to be analysed is in this connection determined by the total length of the sequence to be investigated, the mean length of the NACF sequences and the necessary precision of the sequencing. In the case of a mean length of the NACF sequence determined of 100 NTs, a total length of the sequence to be investigated of 1 Mb and a precision corresponding to the raw sequence determination (i.e. each position is where possible to be sequenced only once), approximately 5 times the quantity of raw sequences is required, i.e. 5 Mb, as distribution of the NACFs takes place randomly over the overall sequence. All in all, 50,000 NACFs must be analysed to cover more than 99% of the total section.
The NACF sequences determined are then assigned to the full sequence with the aid of a commercially available program and any non-conformities detected. Such a program may be based on, for example, the BLAST or FASTA algorithm (“Introduction to computational Biology” 1995 M. S. Waterman Chapman & Hall).

### Example 18

### Composition of Kit for Sequencing Nucleic Acids

In general, one or several kits comprise components (e.g., individual substances, compositions, reaction mixtures) which are necessary for carrying out enzymatic incorporation reactions with modified nuc-macromolecules according to the invention.

The composition of the kits can vary depending on the application, wherein the applications can range from a simple primer-extension reaction up to cyclic sequencing at the single-molecule level.

For instance, the kits which are used for cyclic sequencing can comprise polymerases, modified nuc-macromolecules as well as solutions for the cyclic steps.

Optionally, kits can comprise positive and/or negative controls, instructions for carrying out methods.

Optionally, kits can comprise materials and reagents for preparing components of the kit for biochemical reactions or for preparing the genetic material, e.g., solid phase for material preparation, solid phase for polymerase application, ultrafiltration membrane for rebuffering modified nuc-macromolecules.

The kit components are usually provided in conventional reaction vessels, and the volume of the vessels can vary between 0.2 ml and 1 l. Vessel arrays, e.g. microtiter plates, can be loaded with components, making it possible to feed reagents automatically.

A kit can comprise the following components:
One or more polymerases from the following list: Klenow fragment polymerase, Klenow exo-minus fragment, T7 DNA polymerase, Sequenase 2™, Taq Polymerase, Vent™ polymerase, Deep Vent™ polymerase, Vent™ exo-minus DNA polymerase, Deep Vent™ exo-minus DNA polymerase, Pwo DNA polymerase, reverse transcriptases: e.g. Moloney murine lekemia virus (M-MLV), Rous sarcoma virus (RSV), avian myeloblastosis virus (AMV), Rous-associated virus (RAV), myeloblastosis-associated virus (MAV), human-immunodeficiency virus (HIV). Preferably, polymerases are provided in a storage solution. This storage solution can comprise for example the following substances:
   Buffer Tris-HCl, HEPES, borate, phosphate, acetate (concentrations range for example between 10 mM and 200 mM)
   Salts, e.g. NaCl, KCl, NH₄Cl, concentrations ranging between 10 mM and 500 mM.
   PEG or another inert polymer, e.g. Mowiol, in concentrations between 1 to 50% (w/v)
   Glycerol in concentrations between 1% and 70%
   Reducing agents, e.g. DTT in concentrations between 0.1 and 50 mM
   Further substances contributing to the stability of the enzyme can be contained in a storage solution. Examples of such substances are known; see descriptions of products by enzyme manufacturers, e.g., Promega, Invitrogen, Roche etc.
modified nuc-macromolecules (nucleotide analogs) can be provided as an acid or as salts (e.g., sodium, potassium, ammonium or lithium can be used as ions). The modified nuc-macromolecules can be provided in dried form or in form of a solution, e.g., in water or in a buffer, e.g., Tris-HCl, HEPES, borate, phosphate, acetate, or in a storage solution which can comprise the following components individually or in combination:
   Buffer Tris-HCl, HEPES, borate, phosphate, acetate (in concentrations ranging for example between 10 mM and 200 mM)
   Salts, e.g. NaCl, KCl, NH₄Cl, MgCl₂
   PEG or another inert polymer, e.g. Mowiol, in concentrations between 1 to 20% (w/v)
   Glycerol in concentrations between 1% and 50%
   Marker or marker units of modified nuc-macromolecules, in particular in embodiments in which there is an affine connection between the linker and marker or marker units and the core component.
Buffer compositions for enzymatic reaction, cleaving off, blocking, detection, washing steps:
   Cleaving reagents, provided, e.g., as concentrated buffered solution. For instance, DTT or TCEP in embodiments in which the linker comprises a cleavable disulfide bridge.
   Modifying reagents provided, e.g., as concentrated buffered solution. For example, iodacetamide or iodacetate in embodiments in which the linker has a mercapto group after the cleavage.
Reagents for detection, signal-giving marker units (for embodiments in which the marker of modified nuc-macromolecules has a signal-transmitting function)
   For instance, modified nuc-macromolecules comprise one or several biotin molecules. In this case, e.g., signal-giving streptavidin conjugates can be bonded to such modified nuc-macromolecules prior to the detection step (see paragraph signal-giving marker units).
   For instance, modified nuc-macromolecules comprise streptavidin having free valences for the binding of biotin. In this case, structures comprising biotin can be bonded to the modified nuc-macromolecule prior to the detection step (see paragraph signal-giving marker units).
   Blocking reagents: A kit can comprise different substances to suppress an unspecific adsorption of modified nuc-macromolecules to the surfaces of the solid phase, e.g., acetylated BSA or PEG (2000 to PEG 10,000) or Mowiol or similar polymers which are neutral towards the enzymatic reaction.
Device and means for the preparation of modified nuc-macromolecules for the sequencing reaction. The nucleotide analogs with macromolecular sterically demanding ligands can be purified from the storage buffer by ultrafiltration before use. Devices for ultrafiltration with MWCO of 50,000 Da (available, e.g., from Millipore or Sigma-Aldrich) are suitable for this. This can eliminate not only the storage buffer, but also any decomposition products that may be present. After the one-time centrifugation, the modified nuc-macromolecules are dissolved in the freshly prepared incorporation buffer.
Means for the preparation of polymerases for the sequencing reaction.

Optionally, it is possible to purify polymerases of the manufacturer's solution. For instance, the purification of the polymerases can be conducted via absorption on paramagnetic particles loaded with nucleic acids (e.g., oligonucleotides bound to streptavidin-loaded paramagnetic beads, Promega). After the binding of polymerases to the nucleic acids, the solid phase is washed with incorporation buffer. The application of the polymerases into the reaction can occur either directly with the solid phase, or the polymerases can be liberated from the solid phase by using solutions with higher salt concentration. Instead of binding to the nucleic acids, binding to an anion exchanger, e.g., DEAE cellulose, can be carried out (batch-isolation method for rebuffering proteins). Also a gel filtration (e.g., with Sephadex 25) or an ultrafiltration can be used for the buffer exchange. Other methods for the buffer exchange should seem obvious to a person skilled in the art.
Nucleotides without macromolecular steric obstacle (e.g., dATP, dGTP, dCTP, dTTP, dUTP, dITP) or irreversible terminators (e.g., ddATP, ddGTP, ddCTP, ddTTP, ddUTP)

The object of the invention is furthermore a kit for carrying out the method of sequencing nucleic acid chains and comprising a reaction surface, solutions required for performing the reaction, one or several polymerases, and modified nuc-macromolecules, one to four of which are labeled with fluorescence dyes, wherein modified nuc-macromolecules are structurally modified in such a manner that the polymerase, after such a modified nuc-macromolecule has been incorporated into a growing complement strand, is not capable of incorporating another modified nuc-macromolecule into the same strand, wherein the marker is cleavable and the structural modification is a cleavable macromolecular sterically demanding ligand. Preferably, the nucleotides are the above modified nuc-macromolecules according to the invention.

According to a special embodiment, the kit further comprises reagents necessary, for the preparation of single-stranded nucleic acid from double-stranded nucleic acid, single-stranded nucleic acid molecules which are introduced as a PBS (primer binding site) into the NACFs, oligonucleotide primers, and reagents and/or wash solutions needed to cleave-off the fluorescent dyes and sterically demanding ligands.

All publications, patents and patent applications which were cited here are incorporated into this application in full scope (even if this was not explicitly stated for the individual citation) and are subject to the regulations for “incorporated by reference” for all purposes in the USA according to the USPTO.

### LEGENDS FOR FIGURES

FIG. 8

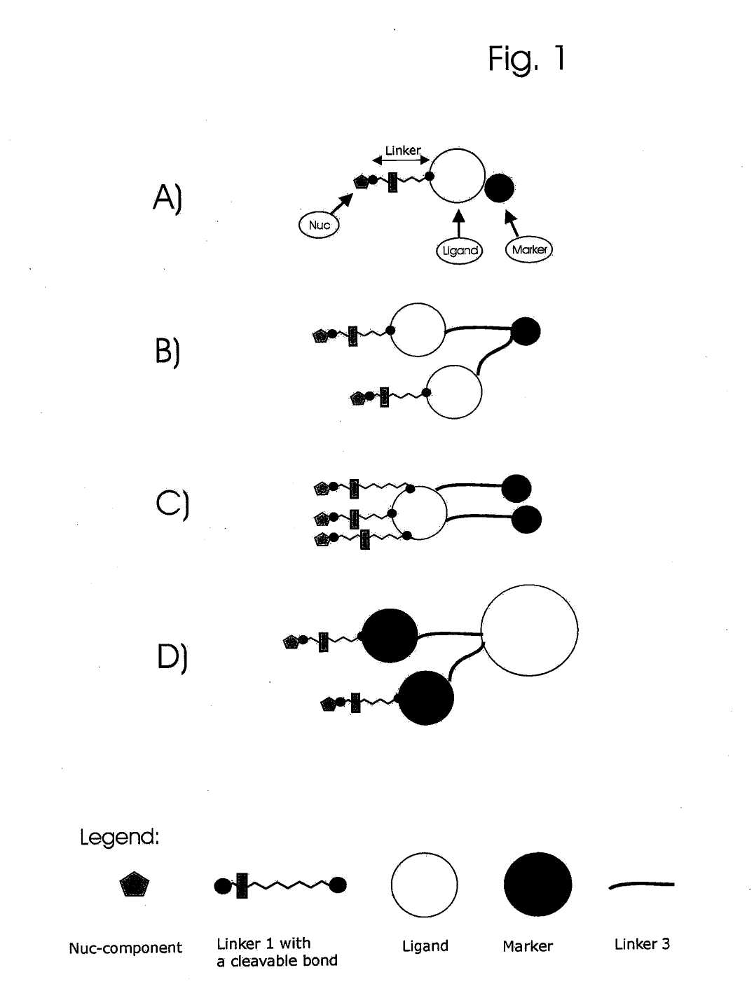

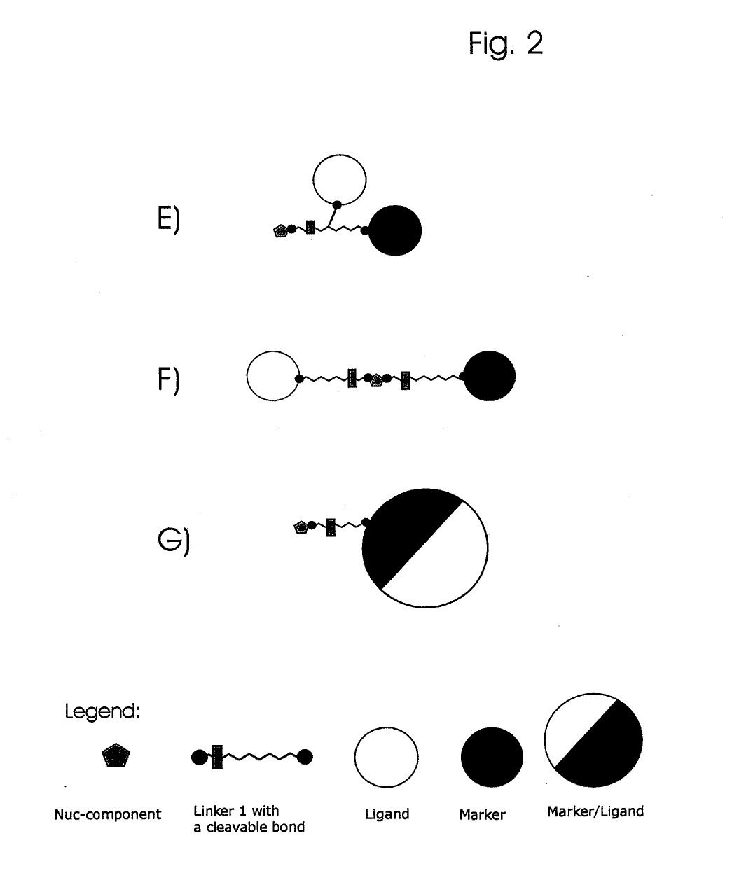

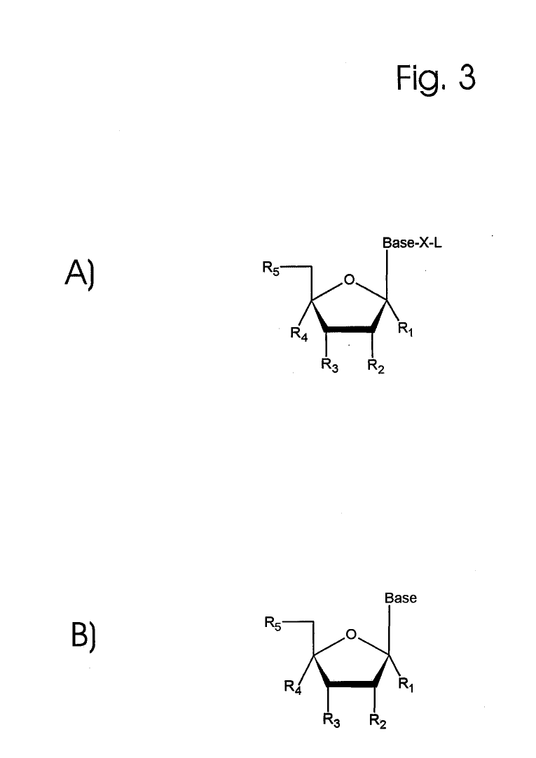

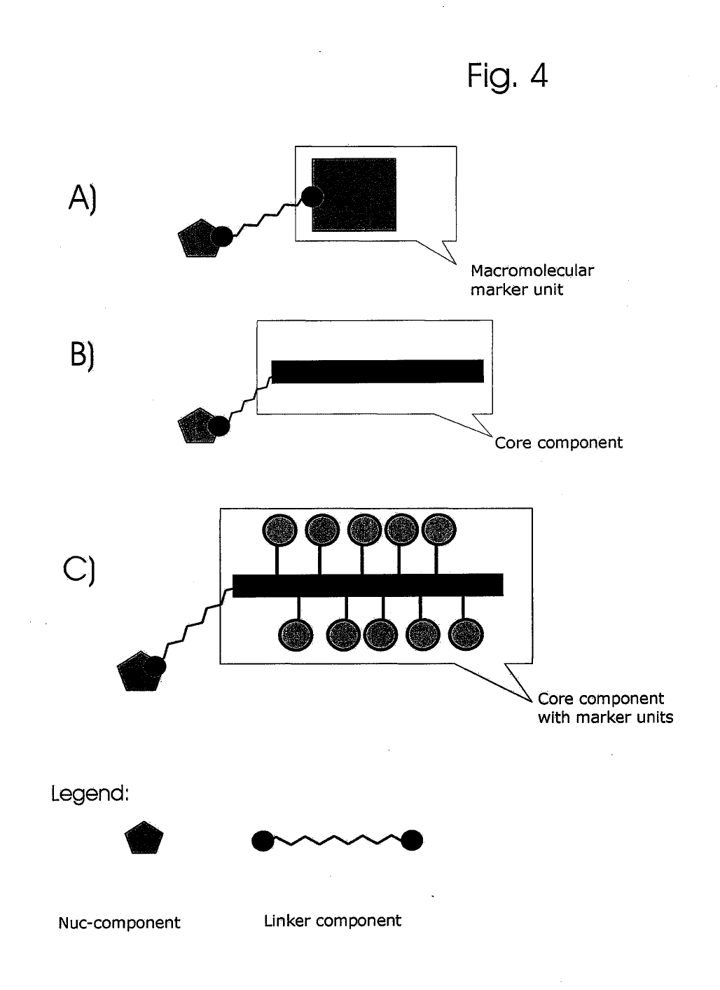

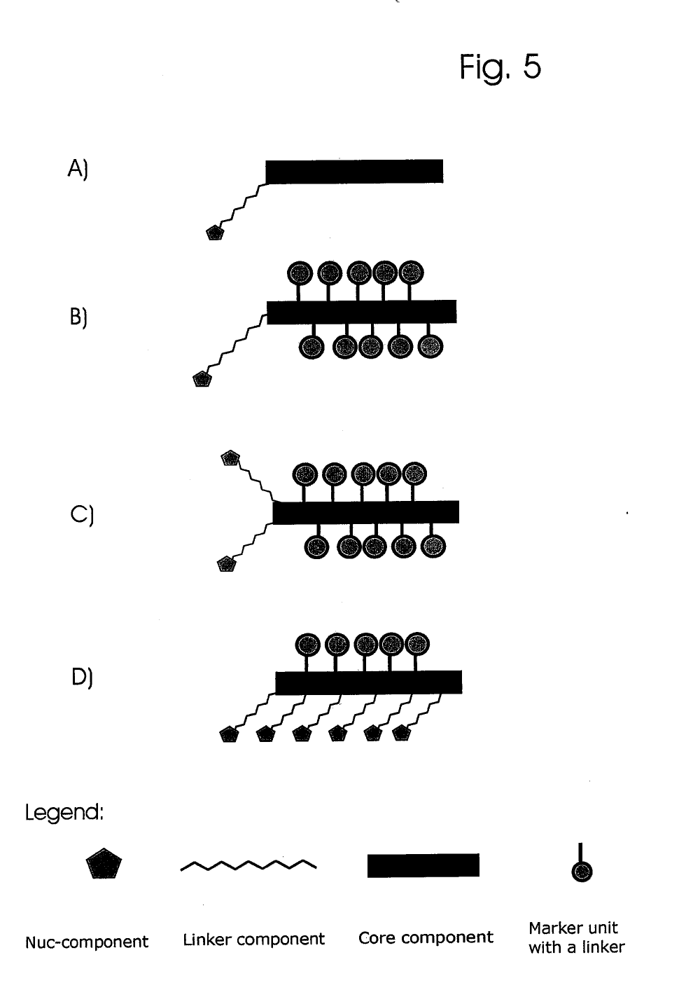

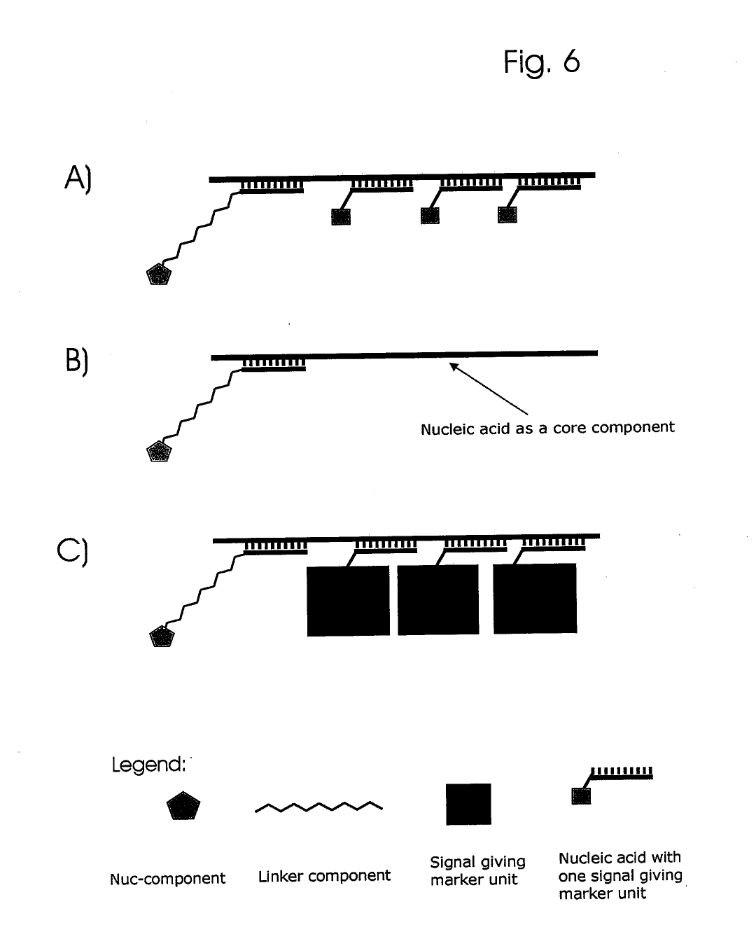

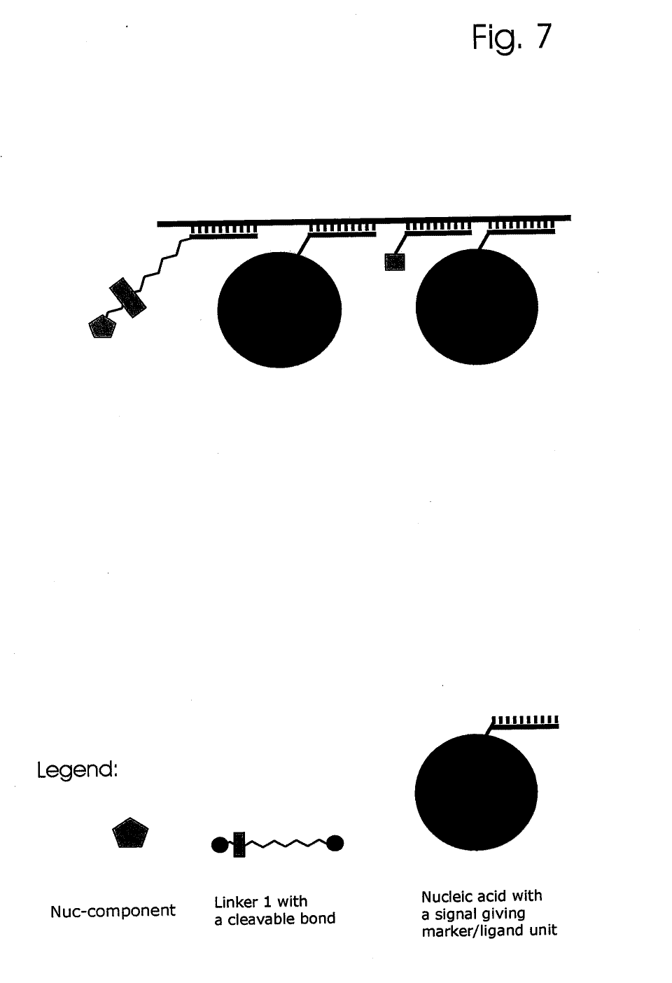

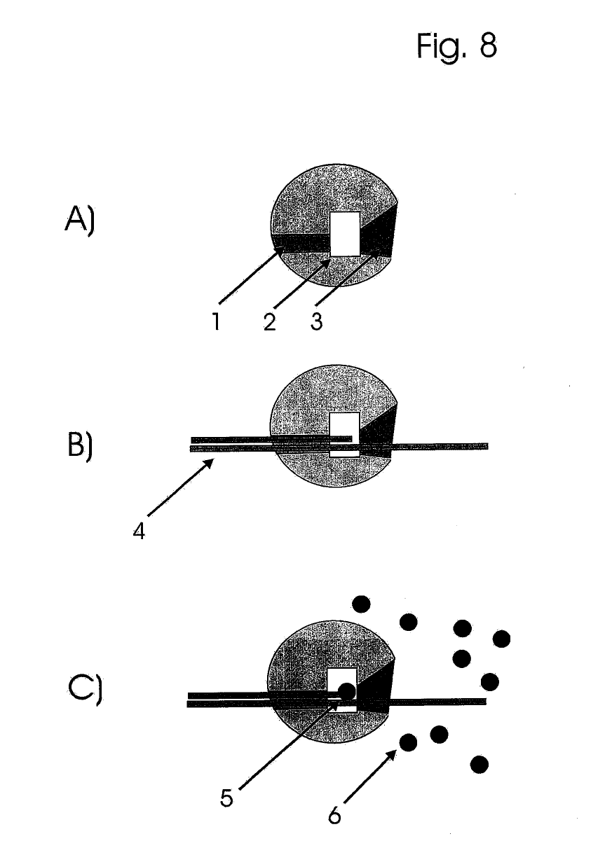

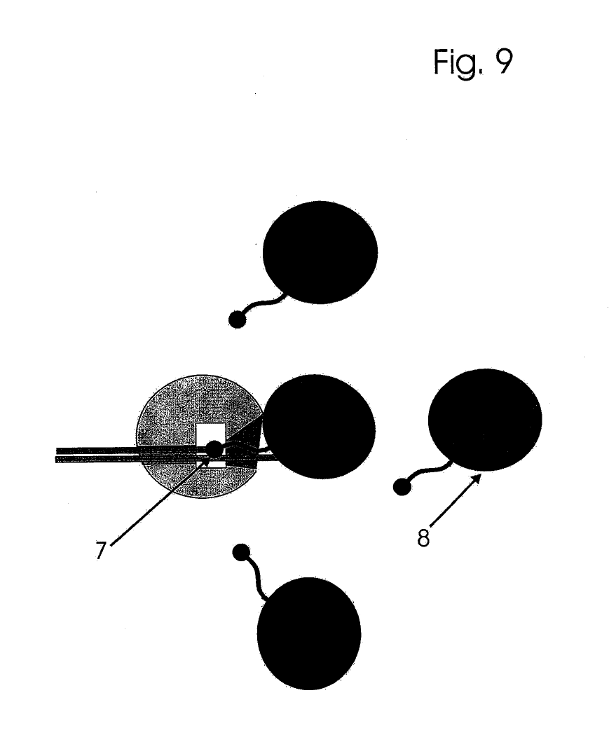

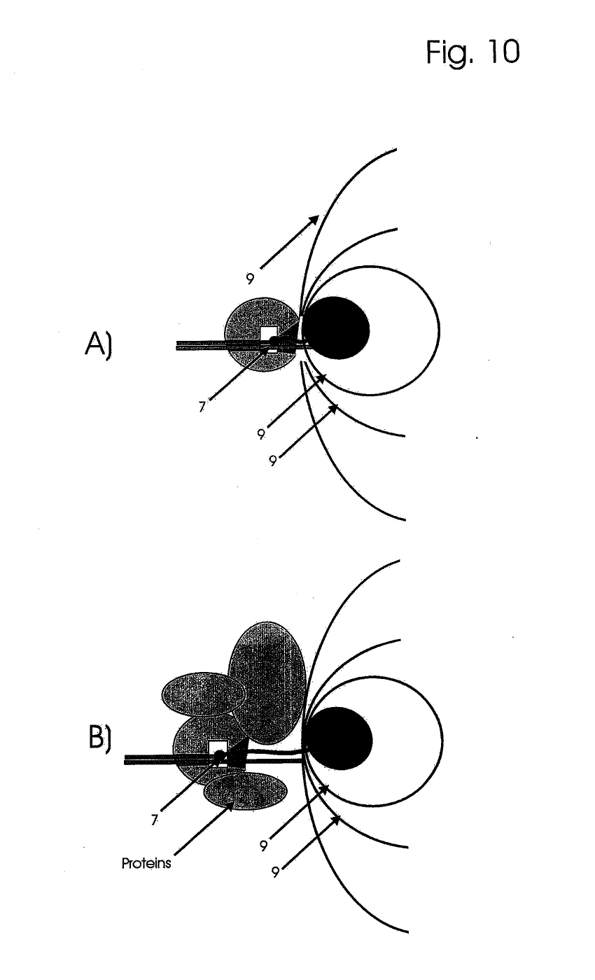

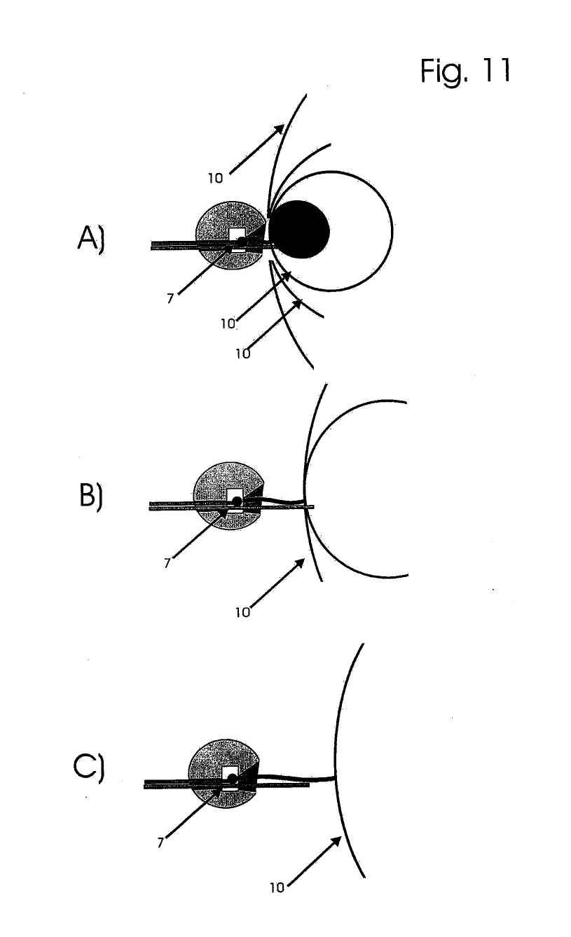

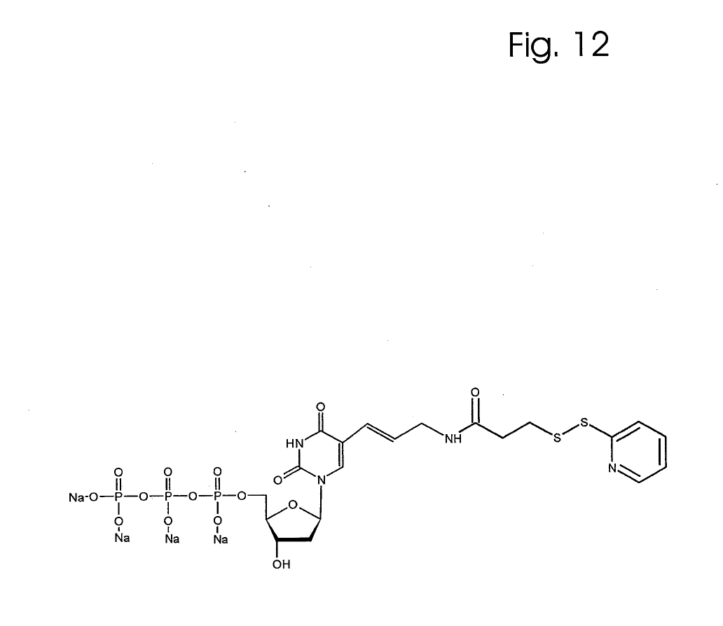

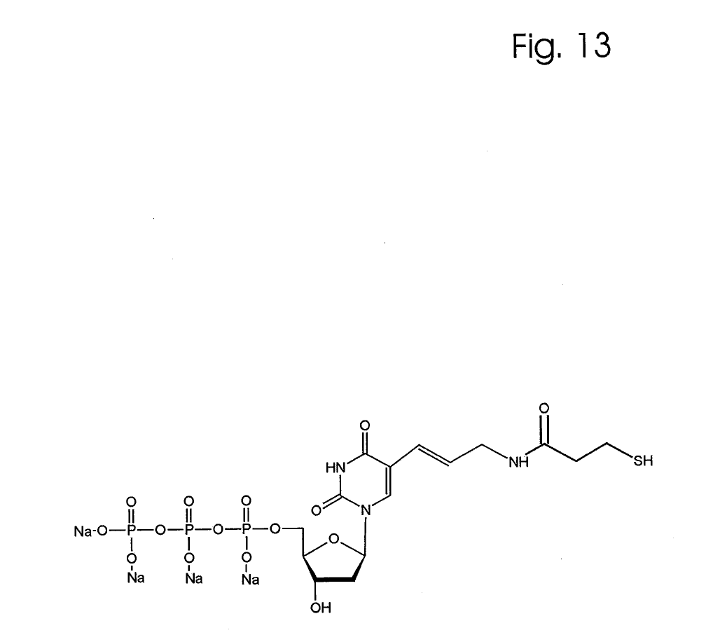

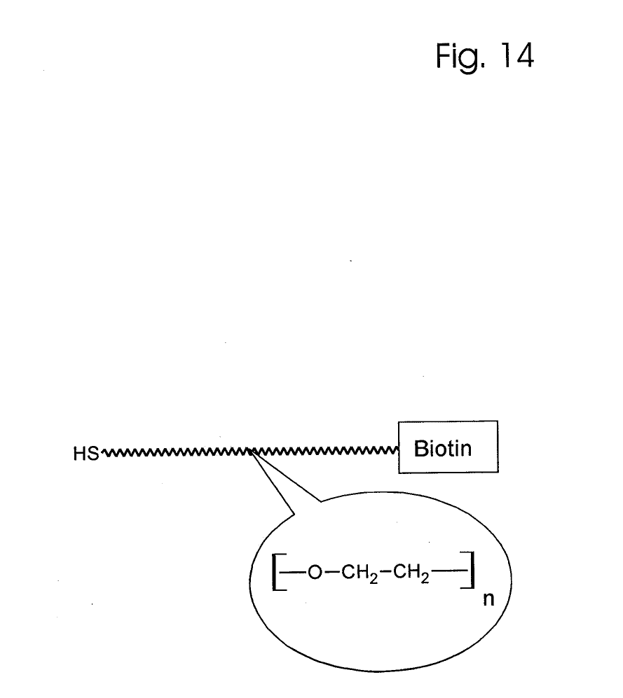

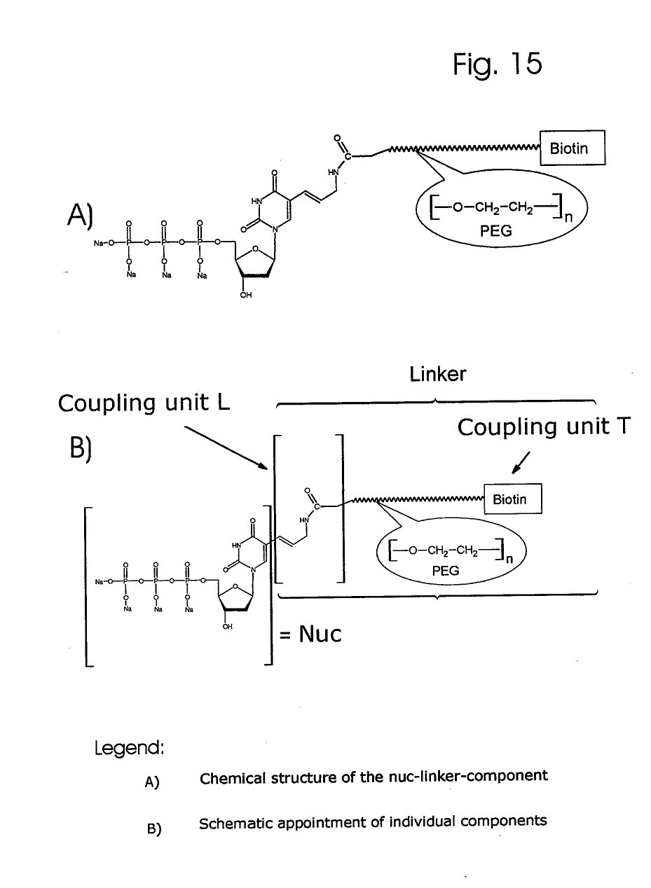

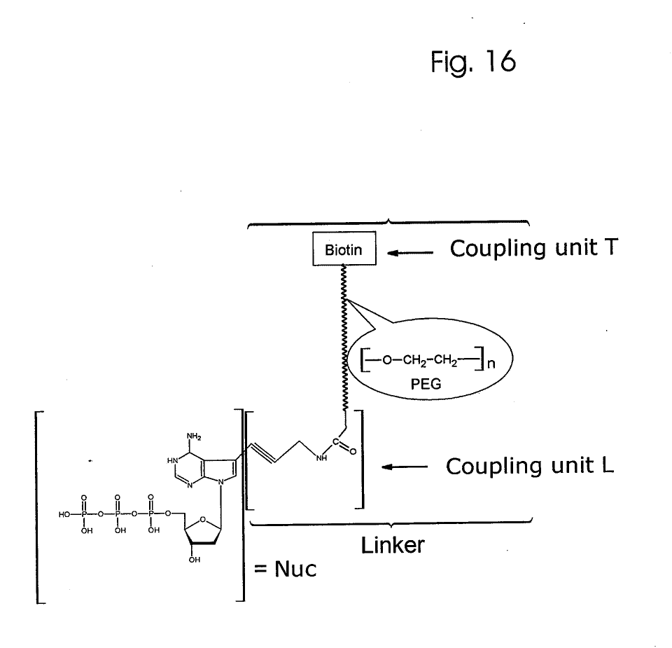

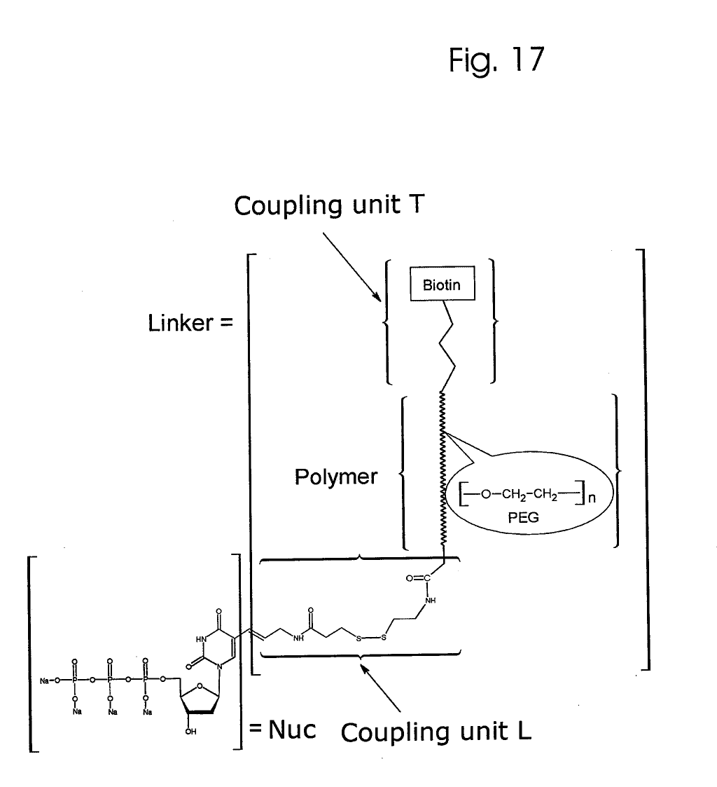

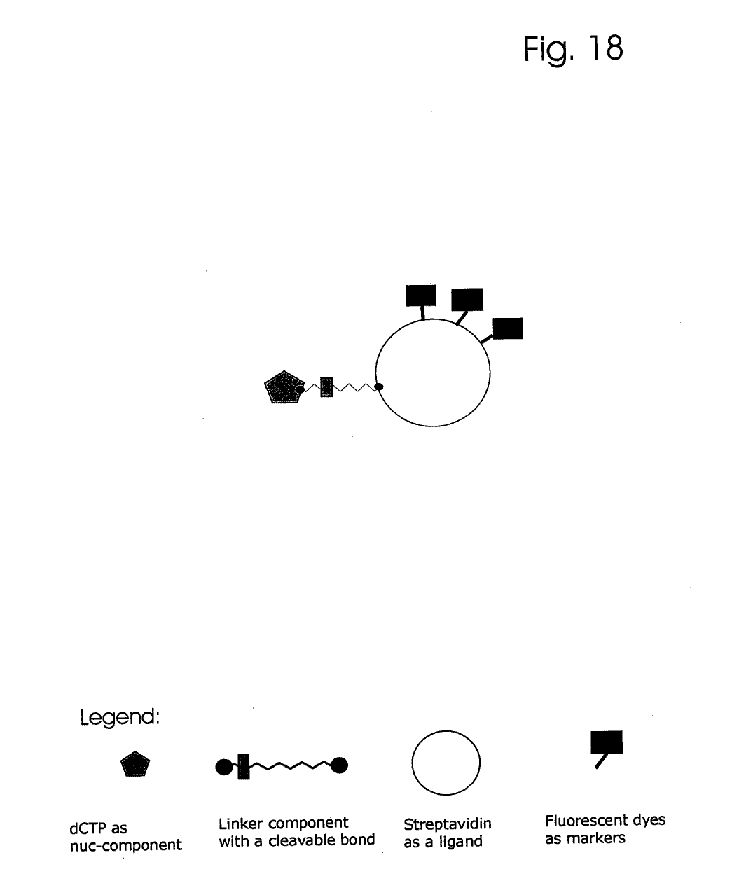

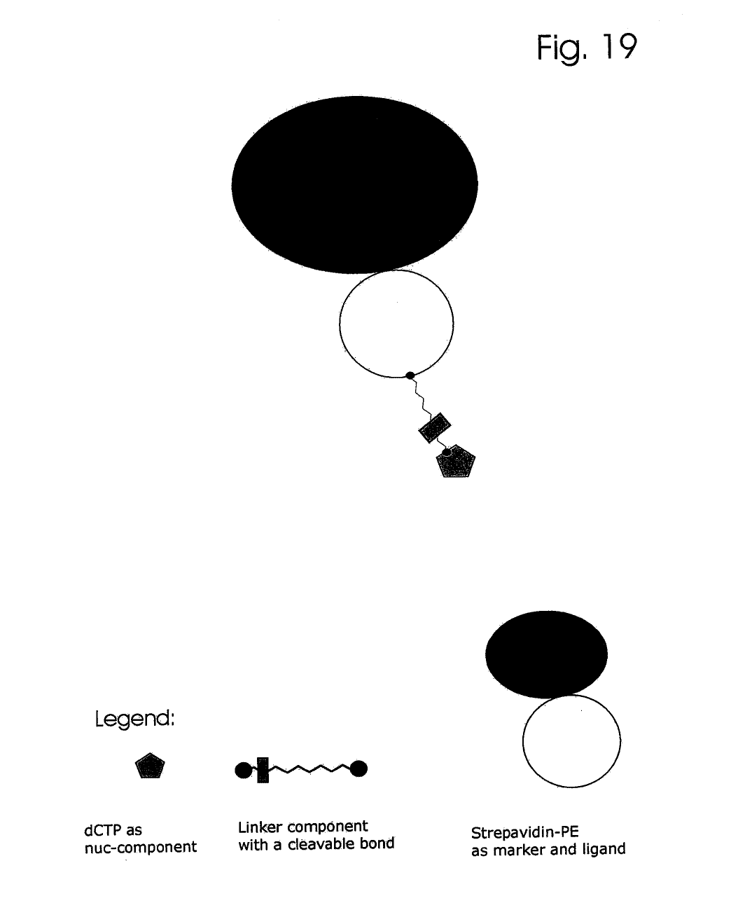

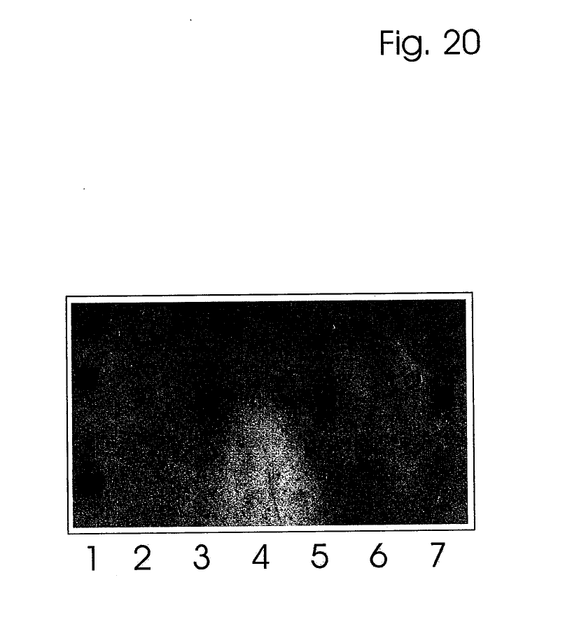

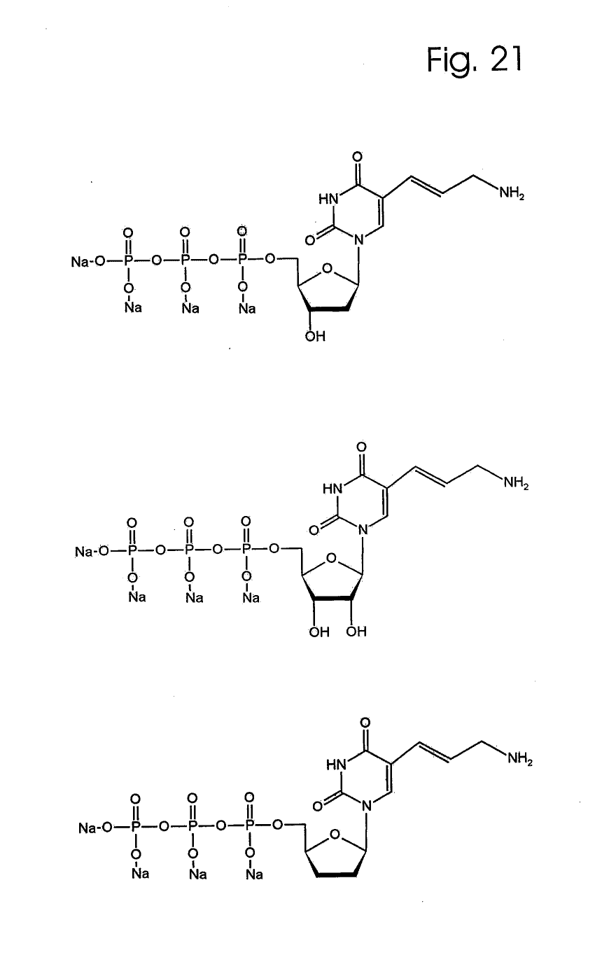

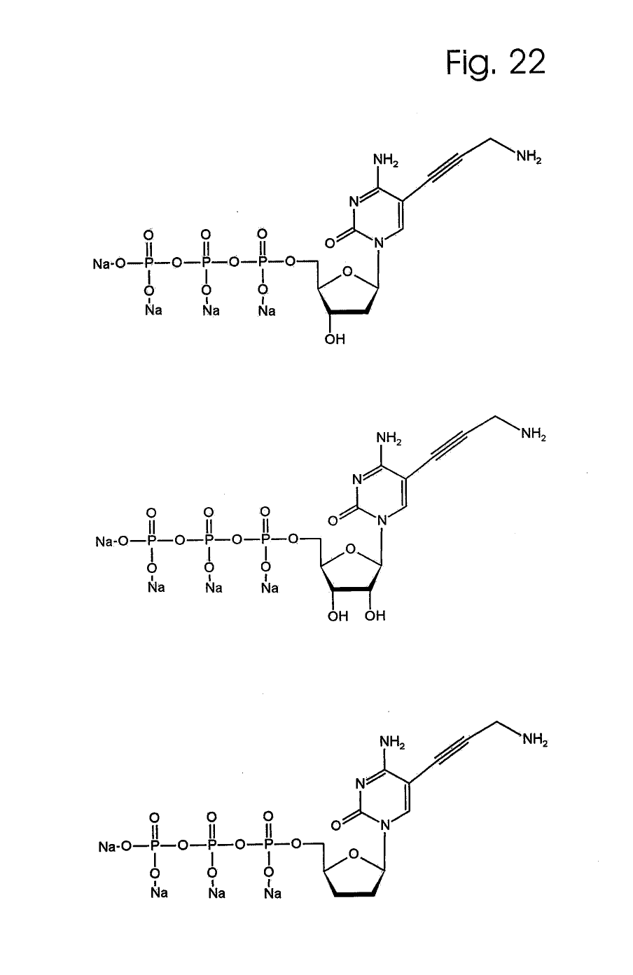

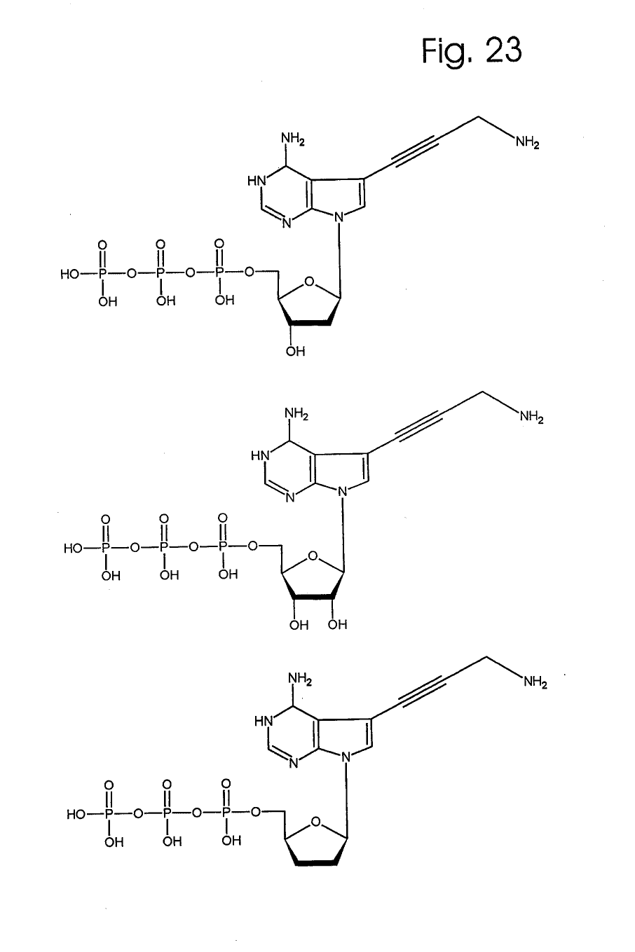

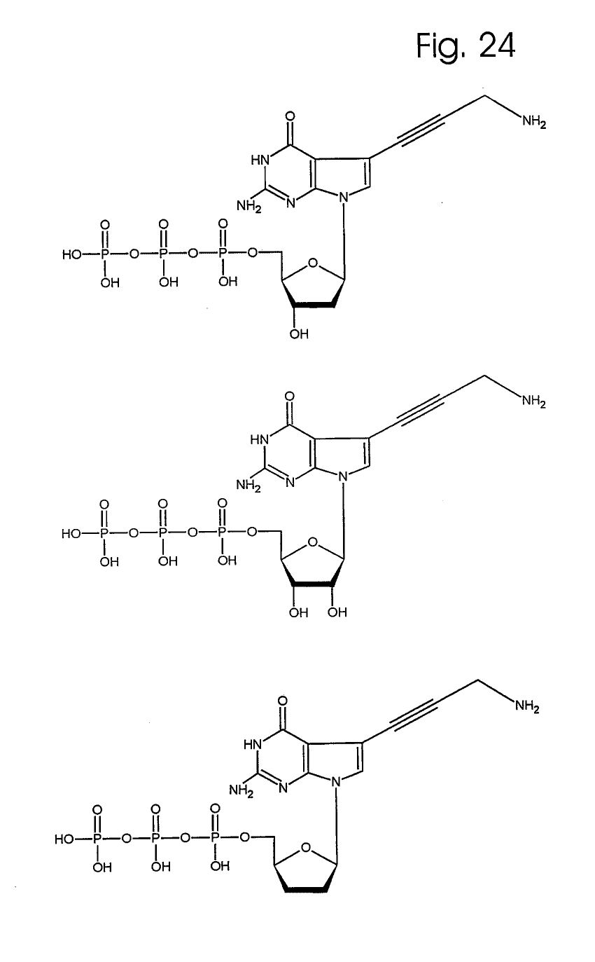

A) Schematic Representation of a Polymerase
DNA binding site (1) of the polymerase (binding of primer and template)
The active center of the polymerase for the coupling of nucleotides to the primer (2)
nucleotide binding site of the polymerase (3)

B) Schematic Representation of a Complex of an Extendable Polymerase-Primer Template: Template-Primer (4)

C) Schematic Representation of the Complex with an Incorporated Nucleotide in the Active Center
An unmodified nucleoside monophosphate coupled to the primer (5)
Free unmodified nucleoside triphosphates (6)

The incorporated nucleotide carries no modification. Free nucleotides have an unobstructed access to the nucleotide binding site of the polymerase

FIG. 9

Schematic Representation of the Complex with an Incorporated Nucleotide in the Active Center
A modified nuc-macromolecule coupled to the primer (7).
Free modified nuc-macromolecules (8) (schematic representation of the nucleotide component, the linker and the sterically demanding ligand).
The incorporated, modified nuc-macromolecule carries a macromolecular sterically demanding ligand. Free modified nuc-macromolecules have no free access to the nucleotide-binding center of the polymerase. The sterically demanding ligand does not permit other ligands into the vicinity of this center of the polymerase. With an appropriately selected linker length between the nucleotide unit and the steric ligand, no other modified nuc-macromolecule can be incorporated. The linker is shown schematically stretched in full length.

FIG. 10

A) Schematic Representation of the Primer-Template-Complex with an Incorporated Nucleotide Component in the Active Center
The sterically demanding ligand of the modified nuc-macromolecule claims a space in the immediate vicinity of the polymerase. The lines (9) schematically show the claimed space.

B) A Change in the Spatial Relationships Around the Polymerase, for Instance, after Other Proteins have been Bonded to the DNA or Polymerase, May Possibly Lead to Necessary Adjustments in the Linker Length Between the Nucleotide Component and the Steric Obstacle.

FIG. 11

Schematic Representation of the Primer-Template-Complex with an Incorporated Nucleotide Component in the Active Center
The longer the linker between the nucleotide component and the macromolecular sterically demanding ligand, the larger the sterically demanding ligand should be for the obstruction of further synthesis. The smaller ligands can lose their effect as the linker length between the nucleotide component and the macromolecular sterically demanding ligand increases.
A potential sterically demanding ligand (10) which has a restraining effect on the synthesis.

FIG. 20

An image with signals originating from cyclic synthesis steps in example 15

Line 1: ladder (dC-analog (upper band) and oligonucleotide-3, labeled with Cy3 dye at 3′ ends (lower band)
Line 2: specimen 1 (control for an unspecific binding of dC-analog to the solid phase)
Line 3: specimen 2 (incorporation of the 1^{st} dC-analog)
Line 4: specimen 3 (cleaving-off of sterically demanding ligands with marker)
Line 5: specimen 4 (incorporation of the 2^{nd} dC-analog)
Line 6: specimen 5 (cleaving-off of sterically demanding ligands with marker)
Line 7: specimen 6 (incorporation of the 3^{rd} dC-analog)

1: Nucleotide analogs (the modified nuc-macromolecules) comprising the following components: at least one nucleotide component (nuc-component), at least one macromolecular sterically demanding ligand, at least one marker, at least one linker.

2: Nucleotide analogs (the modified nuc-macromolecules) according to claim 1, wherein the linker that is coupled to the nucleotide component is cleavable.

3: A reaction mixture comprising at least one of the nucleotide analogs according to claim 1 or 2.

4: A composition comprising at least one of the nucleotide analogs according to claim 1 or 2.

5: A nucleic acid chain or a mixture of nucleic acid chains comprising at least one of the nucleotide analogs according to claim 1 or 2 as a monomer of the nucleic acid chain, wherein the nucleic acid chains can be in a solution or fixed to a solid phase.

6: A nucleic acid chain or a mixture of nucleic acid chains according to claim 5, wherein these nucleic acid chains have a primer function.

7: Method for enzymatic synthesis of the nucleic acid chains, wherein the nucleotide analogs according to claim 1 or 2 are used.

8: A method for the synthesis of nucleic acid chains comprising the following steps:
Preparation of extendable template-primer complexes
Incubation of these complexes in a reaction solution, which comprises one or several types of polymerases and at least one type of the modified nuc-macromolecules according to claim 2, under conditions which allow for primer extension by a modified nuc-macromolecule, wherein the modified nuc-macromolecule is modified in such a way that its incorporation causes further enzymatic reaction to stop

9: A kit for carrying out enzymatic synthesis of nucleic acid chains comprising the following elements:
One or several kinds of polymerases
At least one of the nucleotide analogs, according to claim 1 or 2

10: A Kit for sequencing nucleic acid chains comprising the following elements:
One or several kinds of polymerases
At least one of the nucleotide analogs according to claim 2

11: A method for sequencing of nucleic acid chains comprising the following steps:
a) Preparation of at least one population of extendable nucleic acid chain-primer complexes (NAC-primer complexes),
b) Incubation of at least one type of the modified nuc-macromolecule according to claim 2 together with at least one type of polymerase with the NAC primer complexes prepared in step (a) under conditions which allow for the incorporation of complementary modified nuc-macromolecules, each type of modified nuc-macromolecule having a distinctive label,
c) Removal of the unincorporated modified nuc-macromolecules from the NAC primer complexes,
d) Detection of the signals from the modified nuc-macromolecules which have been incorporated in the NAC primer complexes,
e) Removal of the linker component and the marker component and the macromolecular sterically demanding ligand from the modified nuc-macromolecules which have been incorporated in the NAC primer complexes,
f) Washing of the NAC-primer complexes,
if necessary, repetition of the steps (b) to (f).

12: A method according to claim 11, wherein the nucleic acid chains are attached to a solid phase in random order, and at least a part of this NAC-primer complex is individually optically addressable

13: of the invention relates to a method according to claim 11 for the parallel sequence analysis of nucleic acid sequences (nucleic acid chains, NACs), in which
fragments (NACFs) of single-stranded NACs with a length of approximately 50 to 1000 nucleotides that may represent overlapping partial sequences of the whole sequence are produced,
the NACFs are bonded to a reaction surface in a random order using a uniform primer or several different primers in the form of NACF-primer complexes, wherein the density of NACF-primer complexes bonded to the surface allows for an optical detection of signals from single incorporated modified nuc-macromolecules,
a cyclical synthesis reaction of the complementary strand of the NACFs is performed using one or more polymerases by
a) adding, to the NACF primer complexes bonded to the surface, a solution containing one or more polymerases and one to four modified nuc-macromolecules according to claim 2 that have a marker component labeled with fluorescent elements, wherein the fluorescent elements, which each are located on the marker component when at least two modified nuc-macromolecules are used simultaneously, are chosen in such a manner that the nuc-macromolecules used can be distinguished from one another by measuring different fluorescent signals, the modified nuc-macromolecules being structurally modified in such a manner that the polymerase is not capable of incorporating another nuc-macromolecule in the same strand after such a modified nuc-macromolecule has been incorporated in a growing complementary strand, the linker component and marker component and macromolecular sterically demanding ligand being removable,
b) incubating the stationary phase obtained in step a) under conditions suitable for extending the complementary strands, the complementary strands each being extended by one modified nuc-macromolecule,
c) washing the stationary phase obtained in step b) under conditions suitable for removing modified nuc-macromolecules that are not incorporated in a complementary strand,
d) detecting the single modified nuc-macromolecules incorporated in complementary strands by measuring the characteristic signal of the respective fluorescent elements, the relative position of the individual fluorescent signals on the reaction surface being determined at the same time,
e) cleaving-off the linker component and marker component and the macromolecular sterically demanding ligand from the modified nuc-components added to the complementary strand in order to produce unlabeled NACFs,
f) washing the stationary phase obtained in step e) under conditions suitable for the removal of the marker component,
repeating steps a) to f), several times if necessary,
the relative position of individual NACF-primer complexes on the reaction surface and the sequence of these NACFs being determined by specific assignment of the fluorescent signals that were detected in the respective positions in step d) during successive cycles to the modified nuc-macromolecules.

14: A method according to claim 13, characterized in that steps a) to f) of the cyclical synthesis reaction are repeated several times, only one type of modified nuc-macromolecule being used in each cycle.

15: A method according to claim 13 characterized in that steps a) to f) of the cyclical synthesis reaction are repeated several times, two types of differently labeled modified nuc-macromolecules being used in each cycle.

16: A method according to claim 13 characterized in that steps a) to f) of the cyclical synthesis reaction are repeated several times, four types of differently labeled modified nuc-macromolecules being used in each cycle.

17: A kit for sequencing method of nucleic acid chains according to one of the claims 8 or 11 to 15 comprising the following elements:
One or several kinds of polymerases,
At least one of the nucleotide analogs according to claim 2,
Solutions for performing cyclic sequencing steps.

18: A kit for sequencing nucleic acid chains according to the method according to one of the claims 8 or 11 to 15 comprising one or several of the following compositions, provided as a solution in concentrated or in diluted form or also as a mixture of dry substances, from the following list:
One or several kinds of the polymerases,
At least one of the nucleotide analogs, according to claim 2,
Solutions for performing cyclic sequencing steps,
Composition for incorporation reaction/extension reaction,
Composition for washing the solid phase after the incorporation reaction,
Composition for optical detection of the signals on the solid phase,
Composition for cleaving-off of the marker and the sterically demanding macromolecular ligand,
Composition for washing the solid phase after the cleaving-off of the marker and the sterically demanding macromolecular ligand,
Composition for blockade of the linker residue,
Composition for washing the solid phase after the blockade of the linker residue,
Composition for binding signal-giving marker units to the marker,
Composition with signal-giving marker units.

19: A kit for sequencing nucleic acid chains according to claim 18 which furthermore comprises one or several elements from the following list:
Composition with unmodified nucleotides (dNTPs or NTPs),
Composition with irreversible terminators (ddNTPs),
Composition with terminal transferase,
Composition with a buffer for transferase reaction,
Composition with a ligase,
Composition of oligonucleotides which, as a uniform primer-binding site, can be ligated to the nucleic acid,
Composition with a buffer for ligase reaction,
Solid phase and reagents for preparing nucleic acid chains for the sequencing,
Solid phase and reagents for preparing polymerase for the sequencing,
Device and reagents for preparing nucleotide analogs according to claim 2 for the sequencing,
Composition with blocking reagents for suppression of unspecific adsorption of labeled molecules,
Solid phase for performing cyclic incorporation reactions.

20: A kit for sequencing method of nucleic acid chains according to one of the claims 9, 10, 17, 18 or 19 which comprises one or more polymerases from the following list:
Reverse transcriptases: M-MLV, RSV, AMV, RAV, MAV, HIV
DNA polymerases: Klenow fragment DNA Polymerase, Klenow fragment exo-minus DNA Polymerase, T7 DNA polymerase, Sequenase 2, vent DNA polymerase, vent exo-minus DNA polymerase, Deep Vent DNA polymerase, Deep Vent exo-minus DNA polymerase, Taq DNA polymerase, Tli DNA polymerase, Pwo DNA polymerase, ThermoSequenase DNA polymerase, Pfu DNA polymerase.

21: A kit for sequencing nucleic acid chains according to one of the claims 9, 10, 17, 18 or 19, wherein the components of the compositions are already mixed or are provided as substances in separated form.

22: A kit for sequencing nucleic acid chains according to one of the claims 9, 10, 17, 18 or 19 which comprises one or more solid phases for the performance of cyclic sequencing steps from the following list:
A planar, transparent solid phase,
A planar, transparent solid phase which is provided as a component of a flow-cell or a chip,
A solid phase in form of nano- or microbeads,
A solid phase in form of nano- or microbeads which are paramagnetic,
Solid phase prepared according to patent application DE 101 49 786,
Solid phase prepared according to patent application DE 10 2004 025 744.

23: A method for the synthesis of nucleic acid chains which comprises the following steps:
a) Preparation of extendable primer-template complexes,
b) Incorporation reaction: Incubation of these complexes in a reaction solution containing one or more kinds of polymerase and of at least one type of the modified nuc-macromolecule according to claim 2 under conditions which allow a primer extension by one modified nuc-macromolecule, wherein the modified nuc-macromolecule is modified in such a way that its incorporation causes further enzymatic synthesis to stop,
c) Incubation of the primer-template complexes under conditions which allow for separation of the said primer with incorporated nucleotide analogs from the template,
d) If necessary, repetition of the steps (b) to (c),
e) Application of the obtained labeled primer to a separation medium or in a separation process,
f) Optionally, identification of the type of the nucleotide analog incorporated.
The cyclic steps can be repeated several times, for instance, 2 to 10 times, 10 to 20 times, 20 to 100 times or 100 to 500 times. The identification of the incorporated nucleotide analogs is accomplished by means of the marker.

24: A method for the synthesis of nucleic acid chains comprising the following steps:
a) Preparation of extendable primer-template complexes having addressable positions,
b) Incorporation reaction: Incubation of these complexes in a reaction solution, containing one or more kinds of polymerase and of at least one type of the modified nuc-macromolecules according to claim 2 under conditions which allow a primer extension by one modified nuc-macromolecule, wherein the modified nuc-macromolecule is modified in such a way that its incorporation causes further enzymatic synthesis to stop,
c) Optionally, use of purification steps for template-primer complexes.
d) optionally, identification of the type of incorporated nucleotide analog by detecting marker characteristics, wherein a positional assignment of signals to particular primer-template complexes may be done.
e) Removal of the terminating macromolecular sterically demanding ligand and optionally the marker,
f) Optionally, use of purification steps for template-primer complexes,
g) if necessary, repetition of the steps (b) to (f) and subsequent analysis of the signals identified from incorporated nucleotide analogs.
The cyclic steps can be repeated several times, for instance, 2 to 10 times, 10 to 20 times, 20 to 100 times or 100 to 500 times. The identification of the incorporated nucleotide analogs is accomplished by means of the marker.

25: Nucleotide analogs (modified nuc-macromolecules) with the composition according to claim 1 or 2 comprising the following arrangments of components:
(Nuc-Linker 1)ₙ-(Ligand)ₖ-(Marker)ₘ
(Nuc-Linker 1)ₙ-(Ligand-Linker 3)ₖ-(Marker)ₘ
(Nuc-Linker 1)ₙ-(Ligand)ₖ-(Linker 3-Marker)ₘ
(Nuc-Linker 1)ₙ-(Marker)ₘ-(Ligand)ₖ
(Nuc-Linker 1-Ligand)ₙ-(Marker)ₘ
(Ligand-Linker 2-Nuc-Linker 1)ₙ-(Marker)ₘ
(Nuc-Linker 1)ₙ-(Marker/Ligand)ₘ
(Nuk-Linker 1-Ligand)ₙ-(Marker)ₙ-(Linker 1-Nuk)ₙ
wherein:
Nuc—is a nuc-component
Linker—is a linker component, wherein linker 1 or linker 2 or linker 3 can have identical or different structures
Marker—is a marker component
Ligand—is a macromolecular sterically demanding ligand
Marker/ligand—is a structure that has properties both of a marker and of a macromolecular, sterically demanding ligand
n—is a positive integer from 1 to 100000
m—is a positive integer from 1 to 1000
k—is a positive integer from 1 to 1000
In one embodiment, the structure comprises the following distribution within the molecule: (n)≧(m)≧(k), wherein individual numbers can be varied independently of one another. In a further embodiment, the structure comprises the following distribution: (n)>(m)>(k), wherein individual figures can be varied independently of one another. In a further embodiment, the structure comprises the following distribution: (n)=<(m)>(k), wherein individual figures can be varied independently of one another.

26: Nucleotide analogs according to claim 1,2 or 25, wherein the nuc-component comprises the following structures (FIG. 3A), wherein:
Base is selected independently from the group of adenine, or 7-deazaadenine, or guanine, or 7-deazaguanine, or thymine, or cytosine, or uracil, or their modifications, wherein (L) is the linkage between the nuc-component and the linker component (coupling unit L) and X is the coupling position of the coupling unit (L) to the base.
R₁— is H
R₂— is selected independently from the group of H, OH, halogen, NH₂, SH or protected OH group
R₃— is selected independently from the group of H, OH, halogen, PO₃, SH, N₃, NH₂, O—R₃₋₁, P(O)ₘ—R₃₋₁ ((m) is 1 or 2), NH—R₃₋₁, Si—R₃₋₁ wherein R₃₋₁ is a chemically, photochemically or enzymatically cleavable group or comprises one of the following modifications: —CO—Y, —CH₂—O—Y, —CH₂—S—Y, —CH₂—N₃, —CO—O—Y, —CO—S—Y, —CO—NH—Y, —CH₂—CH═CH₂, wherein Y is an alkyl, for instance (CH₂)ₙ—CH₃ wherein n is a number between 0 and 4, or a substituted alkyl, for instance with halogen, hydroxy group, amino group, carboxy group.
R₄— is H or OH
R₅— is selected independently from the group of OH, or a protected OH group, or a monophosphate group, or a diphosphate group, or a triphosphate group, or is an alpha thiotriphosphate group.

27: Nucleotide analogs according to claim 1, 2 or 25, wherein the nuc-component comprises the following structures (FIG. 3B),
Wherein:
Base is selected independently from the group of adenine, or 7-deazaadenine, or guanine, or 7-deazaguanine, or thymine, or cytosine, or uracil, or their modifications capable of enzymatic reactions.
R₁— is H
R₂— is selected independently from the group of H, OH, halogen, NH₂, SH or protected OH group
R₃— is selected independently from the group of O—R₃₋₂-L, P(O)ₘ—R₃₋₂-L and (m) is 1 or 2, NH—R₃₋₂-L, S—R₃₋₂-L, Si—R₃₋₂-L, wherein R₃₋₂ is the coupling position of the linker to the nucleotide and L is the coupling unit (L) of the linker.
R₄— is H or OH
R₅— is selected independently from the group of OH, or a protected OH group, or a monophosphate group, or a diphosphate group, or a triphosphate group, or is an alpha-thiotriphosphate group.

28: Nucleotide analogs according to claim 1, 2 or 25, wherein the nuc-component comprises the following structures (FIG. 3B),
Wherein:
Base is selected independently from the group of adenine, or 7-deazaadenine, or guanine, or 7-deazaguanine, or thymine, or cytosine, or uracil, or their modifications capable of enzymatic reactions.
R₁— is H
R₂— is selected independently from the group of H, OH, halogen, NH₂, SH or protected OH group
R₃— is selected independently from the group of H, OH, halogen, PO₃, SH, NH₂, O—R₃₋₁, P(O)ₘ—R₃₋₁ ((m) is 1 or 2), NH—R₃₋₁, S—R₃₋₁, Si—R₃₋₁ wherein R₃₋₁ is a chemically, photochemically or enzymatically cleavable group.
R₄— is H or OH
R₅— is selected independently from the group of O—R₅₋₁-L, or P—(O)₃—R₅₋₁-L (modified monophosphate group), or P—(O)₃—P—(O)₃—R₅₋₁-L (modified diphosphate group) or P—(O)₃—P—(O)₃—P—(O)₃—R₅₋₁-L (modified triphosphate group), wherein R₅₋₁ is the coupling position of the coupling unit (L) to the nuc-component and coupling unit (L) is a linkage between nuc-component and linker-component.

29: Nucleotide analogs according to claims 26 to 28, wherein the coupling unit (L) of the linker comprises the following structural elements:
R₆—NH—R₇, R₆—O—R₇, R₆—S—R₇, R₆-SS-R₇, R₆—CO—NH—R₇, R₆—NH—CO—R₇, R₆—CO—O—R₇, R₆—O—CO—R₇, R₆—CO—S—R₇, R₆—S—CO—R₇, R₆—P(O)₂—R₇, R₆—Si—R₇, R₆—(CH₂)ₙ—R₇, R₆—(CH₂)ₙ—R₇, R₆-A-(CH₂)ₙ—R₇, R₆—(CH₂)ₙ—B—R₇, R₆—(CH═CH—)ₙ—R₇, R₆-(A-CH═CH—)ₙ—R₇, R₆—(CH═CH—B—)ₙ—R₇, R₆—(CH═CH—CH₂—B—)ₙ—R₇, R₆-A-CH═CH—(CH₂—)ₙ—R₇, R₆—(—CH═CH—CH₂)ₙ—B—R₇, R₆—(C≡C—)ₙ—R₇, R₆-(A-C≡C—)ₙ—R₇, R₆-(A-C≡C—CH₂)ₙ—R₇, R₆—(C≡C—B—)ₙ—R₇, R₆—(C≡C—CH₂—B—)ₙ—R₇, R₆-A-C≡C—(CH₂—)ₙ—R₇, R₆—(—C≡C—CH₂)ₙ—B—R₇, R₆—(—C≡C—CH₂—CH₂)ₙ—B—R₇
wherein R₆ is the nuc-component, R₇ is the rest of the linker, and A and B comprise independently the following structural elements: —NH—, —O—, —S—, -SS-, —CO—NH—, —NH—CO—, —CO—O—, —O—CO—, —CO—S—, —S—CO—, a photolabile group, —P(O)₂—, —Si—, —(CH₂)ₙ—, wherein (n) ranges from 1 to 5,

30: Nucleotide analogs according to claims 25 to 28, wherein the linker-component comprises a water-soluble polymer.

31: Nucleotide analogs according to claim 30, wherein the linker-component comprises water-soluble polymers selected independently from the following group:
polyethylene glycol (PEG), polysaccharides, dextran, polyamides, polypeptides, polyphosphates, polyacetates, polyalkyleneglycoles, copolymers from ethyleneglycol and propyleneglycol, polyolefinic alcohols, polyvinylpyrrolidones, poly(hydroxyalkylmethacrylamides), polyhydroxyalkylmethacrylates, poly(x-hydroxy) acids, polyacrylic acid, polyacrylamide, polyvinylalcohol.

32: Nucleotide analogs according to one of the claims 1, 2 25 to 31, wherein the average length of a linker component ranges between 50 to 100, 100 to 200, 200 to 500, 500 to 1000, 1000 to 2000, 2000 to 10000, 10000 to 100000, 100000 to 500000 atoms (chain atoms).

33: Nucleotide analogs according to one of the claims 1, 2 25 to 32, wherein a marker component having a signal-giving function, a signal-transmitting function, catalytic function or affine function, or function of a macromolecular sterically demanding ligand

34: Nucleotide analogs according to one of the claim 25 or 33, wherein a structural marker unit independently comprises one of the following structural elements: biotin, hapten, radioactive isotope, rare-earth atom, dye, fluorescent dye.

35: Nucleotide analogs according to one of the claims 25 to 33, wherein a structural marker unit independently comprises one of the following elements: nanocrystals or their modifications, proteins or their modifications, nucleic acids or their modifications, particles or their modifications.

36: Nucleotide analogs according to claim 35, wherein a structural marker unit comprises one of the following proteins:
enzymes or their conjugates or modifications,
antibodies or their conjugates or modifications,
streptavidin or its conjugates or modifications,
avidin or its conjugates or modifications

37: Nucleotide analogs according to one of the claims 1, 2, or 25 to 36, wherein a macromolecular sterically demanding ligand comprises the following structures: proteins, dendrimers, nanoparticles, microparticles or their modifications.
